SEQUENCE LISTING (57 sequences)

SEQ ID NO: 1 - DNA, 8712 bp, synthetic construct
  source 1..8712: mol_type = other DNA
ataggcggcgcatgagagaagcccagaccaattacctacccaaaatggagaaagttcacgttgacatcgaggaagacagcccattcctcagagctttgcagcggagcttcccgcattttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaacaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggccgcggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacgcccgccccacgacccgcagcgcccgaccgaaaggagcgcacgaccccatgatcgctagaccatggggtaccgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaacgcgtcga

SEQ ID NO: 2 - DNA, 8712 bp, synthetic construct
  source 1..8712: mol_type = other DNA
ataggcggcgcatgagagaagcccagaccaattacctacccaaaatggagaaagttcacgttgacatcgaggaagacagcccattcctcagagctttgcagcggagcttcccgcattttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacgcccgccccacgacccgcagcgcccgaccgaaaggagcgcacgaccccatgatcgctagaccatggggtaccgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaacgcgtcga

SEQ ID NO: 3 - DNA, 8712 bp, synthetic construct
  source 1..8712: mol_type = other DNA
ataggcggcgcatgagagaagcccagaccaattacctacccaaaatggagaaagttcacgttgacatcgaggaagacagcccattcctcagagctttgcagcggagcttcccgcattttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaacaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacgcccgccccacgacccgcagcgcccgaccgaaaggagcgcacgaccccatgatcgctagaccatggggtaccgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaacgcgtcga

SEQ ID NO: 4 - DNA, 8712 bp, synthetic construct
  source 1..8712: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaacaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggccgcggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacgcccgccccacgacccgcagcgcccgaccgaaaggagcgcacgaccccatgatcgctagaccatggggtaccgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaacgcgtcga

SEQ ID NO: 5 - DNA, 8724 bp, synthetic construct
  source 1..8724: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggcatcattgaaacccctcggggagcaatcaaggtgaccgcccaacctaccgatcacgtggtaggcgaatacttggtattgtcccctcagaccgtcctccgatctcagaagctgtctctcatacacgcattggccgaacaggtgaagacatgcactcataatggcagagctggaagatacgctgtggaggcttacgatgggcgggttttggtgccttccgggtacgcaatcagcccagaggattttcagagtctttccgagagtgcaacaatggtatataacgaacgcgaattcgttaaccgaaaactccatcacatcgctatgcacggaccagccctcaatactgatgaggaatcttatgagcttgttagggcagagaggaccgaacatgaatatgtttacgacgtggaccaaaggcgatgttgcaagaaggaagaagctgcaggccttgttctcgtaggtgatcttactaacccaccttatcacgagtttgcttacgaaggcttgaaaatacggcctgcatgtccttataagatagccgtaattggcgtattcggagtacccggatccggcaagagtgccataattaaaaatcttgtgactaggcaggacctggtgaccagcggcaaaaaggaaaactgtcaggagatcacaacagacgttatgcgccagcgcgggcttgagatatccgctcgaacagtcgactctctgttgttgaacggatgtaatcgcccagttgatgtactttatgtcgacgaggcttttgcatgtcacagtgggacccttctcgcactgattgcacttgttcgccctaggcaaaaggtagtattgtgtggagacccaaaacagtgcggtttctttaacatgatgcaaatgaaggtgaattacaaccataatatttgtactcaggtgtaccacaagtcaatttcaagacgctgtacacttcctgttaccgcaatagtatcatccctgcattacgagggtaagatgcgcactacaaatgagtacaacaagccaatagtggttgatactaccggctccacaaagcccgatccaggcgatctcgtactcacctgcttccgcgggtgggtaaagcagctccaaatcgattatcggggatacgaagtcatgaccgcagcagcatcacagggtcttactcgcaaaggggtttatgccgtacgccagaaagtgaatgaaaatcccttgtacgcatcaacatctgagcacgtgaatgtgctgttgacacgcaccgaaggcaagcttgtatggaaaactttgagtggtgatccttggataaagaccctgcagaatccaccaaagggaaatttcaaggccacaatcaaggagtgggaggtcgaacatgcaagtattatggcaggaatatgtagccaccagatgacttttgacacatttcagaataaagcaaacgtttgttgggccaaatccttggttcccattctggaaacagctgggattaaattgaatgatcggcagtggagccaaatcatacaagctttcaaagaagataaagcctattcacccgaagtcgccctcaacgagatatgtacaagaatgtatggagtggatcttgatagcggactcttctctaaaccactcgtctcagtctattatgccgataaccactgggacaatagaccaggtggaaaaatgtttggctttaatccagaagcagcctcaattttggagaggaaataccctttcacaaagggcaaatggaatatcaataagcaaatctgcgtcacaactcggcgaatagaagactttaaccccactactaacattatacctgctaataggagacttccccactcattggtagccgaacatcgccctgttaaaggggagcggatggagtggcttgttaacaagatcaacggccatcatgtgttgctcgtatcagactacaatcttgctctccctacaaaacgcgttacctgggtggctcctctcggtgtccgaggagctgactacacctataacctcgaattgggcctgcctgctaccctcggccgctatgatttggtcgtgataaatatacatacccctttccgaatacaccactatcaacagtgtgtagatcacgcaatgaagttgcagatgttgggtggcgatagtcttaggctgcttaagccaggaggcagcctcctgatccgcgcctacggctacgccgaccggactagcgaacgagttatatgcgtcctgggtagaaaattcagaagcagtcgagctttgaagcctccctgtgttacaagtaacaccgagatgttttttcttttcagtaattttgacaacggacggagaaacttcaccacccatgttatgaacaaccaattgaatgccgccttcgtgggacaggctactagagctggatgcgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacgcccgccccacgacccgcagcgcccgaccgaaaggagcgcacgaccccatgatcgctagaccatggggtaccgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaacgcgtcga

SEQ ID NO: 6 - DNA, 8952 bp, synthetic construct
  source 1..8952: mol_type = other DNA
ataggcggcgcatgagagaagcccagaccaattacctacccaaaatggagaaagttcacgttgacatcgaggaagacagcccattcctcagagctttgcagcggagcttcccgcattttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaacaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggccgcggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacgggatccggcgcgaccaatttttcactgttgaagcaagctggggatgtagaagaaaacccaggccctgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacgcccgccccacgacccgcagcgcccgaccgaaaggagcgcacgaccccatgatcgctagaccatggggtaccgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaacgcgtcga

SEQ ID NO: 7 - DNA, 8773 bp, synthetic construct
  source 1..8773: mol_type = other DNA
ataggcggcgcatgagagaagcccagaccaattacctacccaaaatggagaaagttcacgttgacatcgaggaagacagcccattcctcagagctttgcagcggagcttcccgcattttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaacaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggccgcggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacgggatccggcgcgaccaatttttcactgttgaagcaagctggggatgtagaagaaaacccaggccctgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagttagggtaggcaatggcattgatatagcaagaaaattgaaaacagaaaaagttagggtaagcaatggcatataaccataactgtataacttataacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttcc

SEQ ID NO: 8 - DNA, 8394 bp, synthetic construct
  source 1..8394: mol_type = other DNA
atggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgtatgctacgcaaagaaactggcagcggcctccgggaaggtgctggatagagagatcgcagggaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaagaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggccgttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagtcaacgacgtgaagaagcaccgcggactggacatccaggcaaaaacagtggactccatcctgctaaacgggtgtcgtcgtgccgtggacatcctatatgtggacgaggctttcgcttgccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggtaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagaggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcagattcgacttggtctttgtgaacattcacacggaattcagaatccaccactaccagcagtgtgtcgaccacgccatgaagctgcagatgcttgggggagatgcgctacgactgctaaaacccggcggcagcctcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagggcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccatagacatgaggacggctgtggagttgctcaatgatgacgtggagctgaccacagacttggtgagagtgcacccggacagcagcctggtgggtcgtaagggctacagtaccactgacgggtcgctgtactcgtacttggaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtggacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatcgacgacggaccactcagaccggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcatcctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgagactaggccgcgcgggtgcatatattttctcctcggacactggcagcggacatttacaacaaaaatccgttaggcagcacaatctccagtgcgcacaactggatgcggtcgaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggagagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctaaattggtgcgttaatacacagaattctgattatagcgcactattatagcaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagttagggtaggcaatggcattgatatagcaagaaaattgaaaacagaaaaagttagggtaagcaatggcatataaccataactgtataacttataacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttcc

SEQ ID NO: 9 - DNA, 8559 bp, synthetic construct
  source 1..8559: mol_type = other DNA
atggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgtatgctacgcaaagaaactggcagcggcctccgggaaggtgctggatagagagatcgcagggaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaagaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggccgttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagtcaacgacgtgaagaagcaccgcggactggacatccaggcaaaaacagtggactccatcctgctaaacgggtgtcgtcgtgccgtggacatcctatatgtggacgaggctttcgcttgccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggtaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagaggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcagattcgacttggtctttgtgaacattcacacggaattcagaatccaccactaccagcagtgtgtcgaccacgccatgaagctgcagatgcttgggggagatgcgctacgactgctaaaacccggcggcagcctcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagggcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccatagacatgaggacggctgtggagttgctcaatgatgacgtggagctgaccacagacttggtgagagtgcacccggacagcagcctggtgggtcgtaagggctacagtaccactgacgggtcgctgtactcgtacttggaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtggacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatcgacgacggaccactcagaccggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcatcctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgagactaggccgcgcgggtgcatatattttctcctcggacactggcagcggacatttacaacaaaaatccgttaggcagcacaatctccagtgcgcacaactggatgcggtcgaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggagagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctaaattggtgcgttaatacacagaattctgattatagcgcactattatagcaccatggagttcatcccaacccaaactttttacaataggaggtaccagcctcgaccctggactccgcgctctactatccaaatcattaggcccagaccgcgccctggatccggcgcgaccaatttttcactgttgaagcaagctggggatgtagaagaaaacccaggccctgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagttagggtaggcaatggcattgatatagcaagaaaattgaaaacagaaaaagttagggtaagcaatggcatataaccataactgtataacttataacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttcc

SEQ ID NO: 10 - DNA, 8712 bp, synthetic construct
  source 1..8712: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacgcccgccccacgacccgcagcgcccgaccgaaaggagcgcacgaccccatgatcgctagaccatggggtaccgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaacgcgtcga

SEQ ID NO: 11 - DNA, 8712 bp, synthetic construct
  source 1..8712: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacgcccgccccacgacccgcagcgcccgaccgaaaggagcgcacgaccccatgatcgctagaccatggggtaccgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaacgcgtcga

SEQ ID NO: 12 - DNA, 11380 bp, synthetic construct
  source 1..11380: mol_type = other DNA
ataggcggcgcatgagagaagcccagaccaattacctacccaaaatggagaaagttcacgttgacatcgaggaagacagcccattcctcagagctttgcagcggagcttcccgcattttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaacaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggccgcggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggctctgcctgtgaccgctctcctcctgccccttgccctgctgcttcacgctgcaaggccgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaagacgatcatgagagagaaatattcaaagtgttcgagcacaacaacacctgcaccaagacctcctacaccagctccgaccattgcctctcagcccctgtccctccgcccagaggcatgtcgaccagccgccggaggcgctgtgcatacacggggccttgacttcgcttgcgacatttatatttgggcaccgttagcaggcacatgcggggtcctgctccttagcctcgttattacactctactgcaagcgcggccggaagaagctgctgtacatcttcaagcaacctttcatgagaccagtgcagaccacccaggaggaggatgggtgttcctgtaggttccccgaggaagaagaaggcggctgcgagcttcgggtgaaattctcccgcagcgccgacgcccctgcttacaagcagggacagaatcagctgtacaacgagctgaacctcgggagaagagaggaatacgacgtgctggataagagaaggggacgggatccagagatgggaggcaaaccacggcgcaagaatccacaagagggcctgtacaatgagctccagaaggacaagatggctgaggcctattctgagatcggcatgaagggagagcggaggaggggcaagggccacgacggactgtaccagggcctgagtaccgccactaaagatacatatgatgcactgcatatgcaagcattaccacctagggggggcggtggttccggaggaggtggatctggcggaggagggtcagctctgcctgtgaccgctctcctcctgccccttgccctgctgcttcacgctgcaaggccgaggatgcttctgcatttgagtttgctagctcttggagctgcctacgtgtatgccatccccacagaaattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagtacaacaacacctgcaccaagacctcctacaccagctccgaccattgcctctcagcccctgtccctccgcccagaggcatgtcgaccagccgccggaggcgctgtgcatacacggggccttgacttcgcttgcgacatttatatttgggcaccgttagcaggcacatgcggggtcctgctccttagcctcgttattacactctactgcaagcgcggccggaagaagctgctgtacatcttcaagcaacctttcatgagaccagtgcagaccacccaggaggaggatgggtgttcctgtaggttccccgaggaagaagaaggcggctgcgagcttcgggtgaaattctcccgcagcgccgacgcccctgcttacaagcagggacagaatcagctgtacaacgagctgaacctcgggagaagagaggaatacgacgtgctggataagagaaggggacgggatccagagatgggaggcaaaccacggcgcaagaatccacaagagggcctgtacaatgagctccagaaggacaagatggctgaggcctattctgagatcggcatgaagggagagcggaggaggggcaagggccacgacggactgtaccagggcctgagtaccgccactaaagatacatatgatgcactgcatatgcaagcattaccacctaggtaaggccggcccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcaacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccgcggccgcatgccggccgccgccgcggcggccgtggagatcatcctgctgcaggagatcatcaaaaccctgaacagcctgaccgagcagaaaaccctgtgcaccgagctgaccgtgaccgacatcttcgccgccagcaaaaacaccacagagaaagagaccttctgccgcgccgccaccgtgctgcggcagttctactcccacgacgagaaagaccgccgactgcctgggcgccaccgcgcagcagttccacagacacacaactgatcagattcctgaaacgcctggaccggaacctgtgggggctggccggcctgaactcctgcccggtgaaagaggccaaccagagcacactggagaacttcctggagcggctgaaaaccattatggagaaagaaagcagtaagtgctcgagctagtgacgcccgccccacgacccgcagcgcccgaccgaaaggagcgcacgaccccatgatcgctagaccatggggtaccgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaacgcgtcga

SEQ ID NO: 13 - DNA, 9191 bp, synthetic construct
  source 1..9191: mol_type = other DNA
ataggcggcgcatgagagaagcccagaccaattacctacccaaaatggagaaagttcacgttgacatcgaggaagacagcccattcctcagagctttgcagcggagcttcccgcattttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaacaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggccgcggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggctctgcctgtgaccgctctcctcctgccccttgccctgctgcttcacgctgcaaggccgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaagacgatcatgagagagaaatattcaaagtgttcgagcacaacaacacctgcaccaagacctcctacaccagctccgaccattgcctctcagcccctgtccctccgcccagaggcatgtcgaccagccgccggaggcgctgtgcatacacggggccttgacttcgcttgcgacatttatatttgggcaccgttagcaggcacatgcggggtcctgctccttagcctcgttattacactctactgcaagcgcggccggaagaagctgctgtacatcttcaagcaacctttcatgagaccagtgcagaccacccaggaggaggatgggtgttcctgtaggttccccgaggaagaagaaggcggctgcgagcttcgggtgaaattctcccgcagcgccgacgcccctgcttacaagcagggacagaatcagctgtacaacgagctgaacctcgggagaagagaggaatacgacgtgctggataagagaaggggacgggatccagagatgggaggcaaaccacggcgcaagaatccacaagagggcctgtacaatgagctccagaaggacaagatggctgaggcctattctgagatcggcatgaagggagagcggaggaggggcaagggccacgacggactgtaccagggcctgagtaccgccactaaagatacatatgatgcactgcatatgcaagcattaccacctaggtgacgcccgccccacgacccgcagcgcccgaccgaaaggagcgcacgaccccatgatcgctagaccatggggtaccgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaacgcgtcga

SEQ ID NO: 14 - DNA, 9135 bp, synthetic construct
  source 1..9135: mol_type = other DNA
ataggcggcgcatgagagaagcccagaccaattacctacccaaaatggagaaagttcacgttgacatcgaggaagacagcccattcctcagagctttgcagcggagcttcccgcattttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaacaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggccgcggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggctctgcctgtgaccgctctcctcctgccccttgccctgctgcttcacgctgcaaggccgaggatgcttctgcatttgagtttgctagctcttggagctgcctacgtgtatgccatccccacagaaattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagtacaacaacacctgcaccaagacctcctacaccagctccgaccattgcctctcagcccctgtccctccgcccagaggcatgtcgaccagccgccggaggcgctgtgcatacacggggccttgacttcgcttgcgacatttatatttgggcaccgttagcaggcacatgcggggtcctgctccttagcctcgttattacactctactgcaagcgcggccggaagaagctgctgtacatcttcaagcaacctttcatgagaccagtgcagaccacccaggaggaggatgggtgttcctgtaggttccccgaggaagaagaaggcggctgcgagcttcgggtgaaattctcccgcagcgccgacgcccctgcttacaagcagggacagaatcagctgtacaacgagctgaacctcgggagaagagaggaatacgacgtgctggataagagaaggggacgggatccagagatgggaggcaaaccacggcgcaagaatccacaagagggcctgtacaatgagctccagaaggacaagatggctgaggcctattctgagatcggcatgaagggagagcggaggaggggcaagggccacgacggactgtaccagggcctgagtaccgccactaaagatacatatgatgcactgcatatgcaagcattaccacctaggtgacgcccgccccacgacccgcagcgcccgaccgaaaggagcgcacgaccccatgatcgctagaccatggggtaccgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaacgcgtcga

SEQ ID NO: 15 - DNA, 9462 bp, synthetic construct
  source 1..9462: mol_type = other DNA
ataggcggcgcatgagagaagcccagaccaattacctacccaaaatggagaaagttcacgttgacatcgaggaagacagcccattcctcagagctttgcagcggagcttcccgcattttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaacaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggccgcggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggctctgcctgtgaccgctctcctcctgccccttgccctgctgcttcacgctgcaaggccggacattcagatgacccagactacttctagcctgtccgcctctttgggcgacagagtgacaatcagctgcagagctagtcaggatattagcaagtatctgaactggtaccaacagaagccagacgggacagttaagctcctcatctaccacacttctaggctgcactccggcgtcccatcccgcttttccggaagcggcagcggaactgactatagcctgactatcagcaatcttgaacaggaggatattgccacttacttctgtcagcagggaaacactctgccctacacctttggtggtggaaccaagctggagatcacaggaggtggcggcagtggaggcggaggttcaggcggcggaggaagtgaagtgaagctgcaggaaagtggacccgggctggtcgccccttcccaaagccttagcgtcacctgtactgtctctggagtttctctgcccgactacggggtcagttggatcaggcagcctcctaggaaggggctggaatggcttggcgtgatctggggctctgagaccacatattataactccgccctcaaaagcagactcaccattatcaaagacaatagtaaatcccaggtattccttaagatgaacagtctgcagaccgacgacaccgccatctactactgcgctaaacattactactatggaggctcctatgctatggattactggggacagggtacctctgtgaccgtgagctctacaacaacacctgcaccaagacctcctacaccagctccgaccattgcctctcagcccctgtccctccgcccagaggcatgtcgaccagccgccggaggcgctgtgcatacacggggccttgacttcgcttgcgacatttatatttgggcaccgttagcaggcacatgcggggtcctgctccttagcctcgttattacactctactgcaagcgcggccggaagaagctgctgtacatcttcaagcaacctttcatgagaccagtgcagaccacccaggaggaggatgggtgttcctgtaggttccccgaggaagaagaaggcggctgcgagcttcgggtgaaattctcccgcagcgccgacgcccctgcttacaagcagggacagaatcagctgtacaacgagctgaacctcgggagaagagaggaatacgacgtgctggataagagaaggggacgggatccagagatgggaggcaaaccacggcgcaagaatccacaagagggcctgtacaatgagctccagaaggacaagatggctgaggcctattctgagatcggcatgaagggagagcggaggaggggcaagggccacgacggactgtaccagggcctgagtaccgccactaaagatacatatgatgcactgcatatgcaagcattaccacctaggtgacgcccgccccacgacccgcagcgcccgaccgaaaggagcgcacgaccccatgatcgctagaccatggggtaccgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaacgcgtcga

SEQ ID NO: 16 - DNA, 1187 bp, synthetic construct
  source 1..1187: mol_type = other DNA
atggctctgcctgtgaccgctctcctcctgccccttgccctgctgcttcacgctgcaaggccgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaagacgatcatgagagagaaatattcaaagtgttcgagcacaacaacacctgcaccaagacctcctacaccagctccgaccattgcctctcagcccctgtccctccgcccagaggcatgtcgaccagccgccggaggcgctgtgcatacacggggccttgacttcgcttgcgacatttatatttgggcaccgttagcaggcacatgcggggtcctgctccttagcctcgttattacactctactgcaagcgcggccggaagaagctgctgtacatcttcaagcaacctttcatgagaccagtgcagaccacccaggaggaggatgggtgttcctgtaggttccccgaggaagaagaaggcggctgcgagcttcgggtgaaattctcccgcagcgccgacgcccctgcttacaagcagggacagaatcagctgtacaacgagctgaacctcgggagaagagaggaatacgacgtgctggataagagaaggggacgggatccagagatgggaggcaaaccacggcgcaagaatccacaagagggcctgtacaatgagctccagaaggacaagatggctgaggcctattctgagatcggcatgaagggagagcggaggaggggcaagggccacgacggactgtaccagggcctgagtaccgccactaaagatacatatgatgcactgcatatgcaagcattaccacctagg

SEQ ID NO: 17 - DNA, 1131 bp, synthetic construct
  source 1..1131: mol_type = other DNA
gctctgcctgtgaccgctctcctcctgccccttgccctgctgcttcacgctgcaaggccgaggatgcttctgcatttgagtttgctagctcttggagctgcctacgtgtatgccatccccacagaaattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagtacaacaacacctgcaccaagacctcctacaccagctccgaccattgcctctcagcccctgtccctccgcccagaggcatgtcgaccagccgccggaggcgctgtgcatacacggggccttgacttcgcttgcgacatttatatttgggcaccgttagcaggcacatgcggggtcctgctccttagcctcgttattacactctactgcaagcgcggccggaagaagctgctgtacatcttcaagcaacctttcatgagaccagtgcagaccacccaggaggaggatgggtgttcctgtaggttccccgaggaagaagaaggcggctgcgagcttcgggtgaaattctcccgcagcgccgacgcccctgcttacaagcagggacagaatcagctgtacaacgagctgaacctcgggagaagagaggaatacgacgtgctggataagagaaggggacgggatccagagatgggaggcaaaccacggcgcaagaatccacaagagggcctgtacaatgagctccagaaggacaagatggctgaggcctattctgagatcggcatgaagggagagcggaggaggggcaagggccacgacggactgtaccagggcctgagtaccgccactaaagatacatatgatgcactgcatatgcaagcattaccacctaggtaa

SEQ ID NO: 18 - DNA, 413 bp, synthetic construct
  source 1..413: mol_type = other DNA
atgccggccgccgccgcggcggccgtggagatcatcctgctgcaggagatcatcaaaaccctgaacagcctgaccgagcagaaaaccctgtgcaccgagctgaccgtgaccgacatcttcgccgccagcaaaaacaccacagagaaagagaccttctgccgcgccgccaccgtgctgcggcagttctactcccacgacgagaaagaccgccgactgcctgggcgccaccgcgcagcagttccacagacacacaactgatcagattcctgaaacgcctggaccggaacctgtgggggctggccggcctgaactcctgcccggtgaaagaggccaaccagagcacactggagaacttcctggagcggctgaaaaccattatggagaaagaaagcagtaagtgctcgagctagtga

SEQ ID NO: 19 - DNA, 603 bp, synthetic construct
  source 1..603: mol_type = other DNA
ggccggcccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcaacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccgcggccgc

SEQ ID NO: 20 - DNA, 45 bp, synthetic construct
  source 1..45: mol_type = other DNA
gggggcggtggttccggaggaggtggatctggcggaggagggtca

SEQ ID NO: 21 - DNA, 9191 bp, synthetic construct; identical to SEQ ID NO: 13
  source 1..9191: mol_type = other DNA

SEQ ID NO: 22 - DNA, 1190 bp, synthetic construct
  source 1..1190: mol_type = other DNA
atggctctgcctgtgaccgctctcctcctgccccttgccctgctgcttcacgctgcaaggccgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaagacgatcatgagagagaaatattcaaagtgttcgagcacaacaacacctgcaccaagacctcctacaccagctccgaccattgcctctcagcccctgtccctccgcccagaggcatgtcgaccagccgccggaggcgctgtgcatacacggggccttgacttcgcttgcgacatttatatttgggcaccgttagcaggcacatgcggggtcctgctccttagcctcgttattacactctactgcaagcgcggccggaagaagctgctgtacatcttcaagcaacctttcatgagaccagtgcagaccacccaggaggaggatgggtgttcctgtaggttccccgaggaagaagaaggcggctgcgagcttcgggtgaaattctcccgcagcgccgacgcccctgcttacaagcagggacagaatcagctgtacaacgagctgaacctcgggagaagagaggaatacgacgtgctggataagagaaggggacgggatccagagatgggaggcaaaccacggcgcaagaatccacaagagggcctgtacaatgagctccagaaggacaagatggctgaggcctattctgagatcggcatgaagggagagcggaggaggggcaagggccacgacggactgtaccagggcctgagtaccgccactaaagatacatatgatgcactgcatatgcaagcattaccacctaggtga

SEQ ID NO: 23 - DNA, 9135 bp, synthetic construct; identical to SEQ ID NO: 14
  source 1..9135: mol_type = other DNA

SEQ ID NO: 24 - DNA, 1134 bp, synthetic construct
  source 1..1134: mol_type = other DNA
atggctctgcctgtgaccgctctcctcctgccccttgccctgctgcttcacgctgcaaggccgaggatgcttctgcatttgagtttgctagctcttggagctgcctacgtgtatgccatccccacagaaattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagtacaacaacacctgcaccaagacctcctacaccagctccgaccattgcctctcagcccctgtccctccgcccagaggcatgtcgaccagccgccggaggcgctgtgcatacacggggccttgacttcgcttgcgacatttatatttgggcaccgttagcaggcacatgcggggtcctgctccttagcctcgttattacactctactgcaagcgcggccggaagaagctgctgtacatcttcaagcaacctttcatgagaccagtgcagaccacccaggaggaggatgggtgttcctgtaggttccccgaggaagaagaaggcggctgcgagcttcgggtgaaattctcccgcagcgccgacgcccctgcttacaagcagggacagaatcagctgtacaacgagctgaacctcgggagaagagaggaatacgacgtgctggataagagaaggggacgggatccagagatgggaggcaaaccacggcgcaagaatccacaagagggcctgtacaatgagctccagaaggacaagatggctgaggcctattctgagatcggcatgaagggagagcggaggaggggcaagggccacgacggactgtaccagggcctgagtaccgccactaaagatacatatgatgcactgcatatgcaagcattaccacctaggtga

SEQ ID NO: 25 - DNA, 9462 bp, synthetic construct; identical to SEQ ID NO: 15
  source 1..9462: mol_type = other DNA

SEQ ID NO: 26 - DNA, 1461 bp, synthetic construct
  source 1..1461: mol_type = other DNA
atggctctgcctgtgaccgctctcctcctgccccttgccctgctgcttcacgctgcaaggccggacattcagatgacccagactacttctagcctgtccgcctctttgggcgacagagtgacaatcagctgcagagctagtcaggatattagcaagtatctgaactggtaccaacagaagccagacgggacagttaagctcctcatctaccacacttctaggctgcactccggcgtcccatcccgcttttccggaagcggcagcggaactgactatagcctgactatcagcaatcttgaacaggaggatattgccacttacttctgtcagcagggaaacactctgccctacacctttggtggtggaaccaagctggagatcacaggaggtggcggcagtggaggcggaggttcaggcggcggaggaagtgaagtgaagctgcaggaaagtggacccgggctggtcgccccttcccaaagccttagcgtcacctgtactgtctctggagtttctctgcccgactacggggtcagttggatcaggcagcctcctaggaaggggctggaatggcttggcgtgatctggggctctgagaccacatattataactccgccctcaaaagcagactcaccattatcaaagacaatagtaaatcccaggtattccttaagatgaacagtctgcagaccgacgacaccgccatctactactgcgctaaacattactactatggaggctcctatgctatggattactggggacagggtacctctgtgaccgtgagctctacaacaacacctgcaccaagacctcctacaccagctccgaccattgcctctcagcccctgtccctccgcccagaggcatgtcgaccagccgccggaggcgctgtgcatacacggggccttgacttcgcttgcgacatttatatttgggcaccgttagcaggcacatgcggggtcctgctccttagcctcgttattacactctactgcaagcgcggccggaagaagctgctgtacatcttcaagcaacctttcatgagaccagtgcagaccacccaggaggaggatgggtgttcctgtaggttccccgaggaagaagaaggcggctgcgagcttcgggtgaaattctcccgcagcgccgacgcccctgcttacaagcagggacagaatcagctgtacaacgagctgaacctcgggagaagagaggaatacgacgtgctggataagagaaggggacgggatccagagatgggaggcaaaccacggcgcaagaatccacaagagggcctgtacaatgagctccagaaggacaagatggctgaggcctattctgagatcggcatgaagggagagcggaggaggggcaagggccacgacggactgtaccagggcctgagtaccgccactaaagatacatatgatgcactgcatatgcaagcattaccacctaggtga

SEQ ID NO: 27 - DNA, 34 bp, synthetic construct
  source 1..34: mol_type = other DNA
ataggcggcgcatgagagaagcccagaccaatta

SEQ ID NO: 28 - DNA, 1619 bp, synthetic construct
  source 1..1619: mol_type = other DNA
attacctacccaaaatggagaaagttcacgttgacatcgaggaagacagcccattcctcagagctttgcagcggagcttcccgcattttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggcc

SEQ ID NO: 29 - DNA, 2382 bp, synthetic construct
  source 1..2382: mol_type = other DNA
ggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaacaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgt

SEQ ID NO: 30 - DNA, 1671 bp, synthetic construct
  source 1..1671: mol_type = other DNA
gcaccctcatatcatgtggtgcgaggggatattgccacggccaccgaaggagtgattataaatgctgctaacagcaaaggacaacctggccgcggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgca

SEQ ID NO: 31 - DNA, 1826 bp, synthetic construct
  source 1..1826: mol_type = other DNA
catacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaa

SEQ ID NO: 32 - DNA, 440 bp, synthetic construct
  source 1..440: mol_type = other DNA
cgcccgccccacgacccgcagcgcccgaccgaaaggagcgcacgaccccatgatcgctagaccatggggtaccgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaacgcgtcga

SEQ ID NO: 33 - DNA, 35 bp, synthetic construct
  source 1..35: mol_type = other DNA
cctgaatggactacgacatagtctagtccgccaag

SEQ ID NO: 34 - DNA, 1605 bp, synthetic construct
  source 1..1605: mol_type = other DNA
atggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggcc

SEQ ID NO: 35 - DNA, 2394 bp, synthetic construct
  source 1..2394: mol_type = other DNA
ggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatat

SEQ ID NO: 36 - DNA, 1084 bp, synthetic construct
  source 1..1084: mol_type = other DNA
ggctggggccggcatcattgaaacccctcggggagcaatcaaggtgaccgcccaacctaccgatcacgtggtaggcgaatacttggtattgtcccctcagaccgtcctccgatctcagaagctgtctctcatacacgcattggccgaacaggtgaagacatgcactcataatggcagagctggaagatacgctgtggaggcttacgatgggcgggttttggtgccttccgggtacgcaatcagcccagaggattttcagagtctttccgagagtgcaacaatggtatataacgaacgcgaattcgttaaccgaaaactccatcacatcgctatgcacggaccagccctcaatactgatgaggaatcttatgagcttgttagggcagagaggaccgaacatgaatatgtttacgacgtggaccaaaggcgatgttgcaagaaggaagaagctgcaggccttgttctcgtaggtgatcttactaacccaccttatcacgagtttgcttacgaaggcttgaaaatacggcctgcatgtccttataagatagccgtaattggcgtattcggagtacccggatccggcaagagtgccataattaaaaatcttgtgactaggcaggacctggtgaccagcggcaaaattacctgggtggctcctctcggtgtccgaggagctgactacacctataacctcgaattgggcctgcctgctaccctcggccgctatgatttggtcgtgataaatatacatacccctttccgaatacaccactatcaacagtgtgtagatcacgcaatgaagttgcagatgttgggtggcgatagtcttaggctgcttaagccaggaggcagcctcctgatccgcgcctacggctacgccgaccggactagcgaacgagttatatgcgtcctgggtagaaaattcagaagcagtcgagctttgaagcctccctgtgttacaagtaacaccgagatgttttttcttttcagtaattttgacaacggacggagaaacttcaccacccatgttatgaacaaccaattgaatgccgccttcgtgggacaggctactagagctggatgc

SEQ ID NO: 37 - DNA, 1671 bp, synthetic construct
  source 1..1671: mol_type = other DNA
gcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgca

SEQ ID NO: 38 - DNA, 1671 bp, synthetic construct; identical to SEQ ID NO: 37
  source 1..1671: mol_type = other DNA

SEQ ID NO: 39 - DNA, 1824 bp, synthetic construct
  source 1..1824: mol_type = other DNA
tacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaa

SEQ ID NO: 40 - DNA, 35 bp, synthetic construct; identical to SEQ ID NO: 33
  source 1..35: mol_type = other DNA

SEQ ID NO: 41 - DNA, 365 bp, synthetic construct
  source 1..365: mol_type = other DNA
gtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaacgcgtcga

SEQ ID NO: 42 - DNA, 345 bp, synthetic construct
  source 1..345: mol_type = other DNA
tgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaa

SEQ ID NO: 43 - DNA, 554 bp, synthetic construct
  source 1..554: mol_type = other DNA
gtcaacatcaagttggacatcacctcccacaacgaggactacaccatcgtggaacagtacgaacgcgccgagggccgccactccaccggcggcatggacgagctgtacaagtaacgcccgccccacgacccgcagcgcccgaccgaaaggagcgcacgaccccatgatcgctagaccatggggtaccgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaacgcgtcga

SEQ ID NO: 44 - DNA, 261 bp, synthetic construct
  source 1..261: mol_type = other DNA
gttagggtaggcaatggcattgatatagcaagaaaattgaaaacagaaaaagttagggtaagcaatggcatataaccataactgtataacttataacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttcc

SEQ ID NO: 45 - DNA, 1234 bp, synthetic construct
  source 1..1234: mol_type = other DNA
gttcctctggaagcttcttgaagacaaacaacgtctgtagcgaccctttgcaggcagcggaaccccccacctggcaacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccgcggccgcatgccggccgccgccgcggcggccgtggagatcatcctgctgcaggagatcatcaaaaccctgaacagcctgaccgagcagaaaaccctgtgcaccgagctgaccgtgaccgacatcttcgccgccagcaaaaacaccacagagaaagagaccttctgccgcgccgccaccgtgctgcggcagttctactcccacgacgagaaagaccgccgactgcctgggcgccaccgcgcagcagttccacagacacacaactgatcagattcctgaaacgcctggaccggaacctgtgggggctggccggcctgaactcctgcccggtgaaagaggccaaccagagcacactggagaacttcctggagcggctgaaaaccattatggagaaagaaagcagtaagtgctcgagctagtgacgcccgccccacgacccgcagcgcccgaccgaaaggagcgcacgaccccatgatcgctagaccatggggtaccgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaacgcgtcga

SEQ ID NO: 46 - DNA, 203 bp, synthetic construct
  source 1..203: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacg

SEQ ID NO: 47 - DNA, 66 bp, synthetic construct
  source 1..66: mol_type = other DNA
ggatccggcgcgaccaatttttcactgttgaagcaagctggggatgtagaagaaaacccaggccct

SEQ ID NO: 48 - DNA, 85 bp, synthetic construct
  source 1..85: mol_type = other DNA
atggcggatgtgtgacatacacgacgccaaaagattttgttccagctcctgccacctccgctacgcgagagattaaccacccacg

SEQ ID NO: 49 - DNA, 1611 bp, synthetic construct
  source 1..1611: mol_type = other DNA
atggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgtatgctacgcaaagaaactggcagcggcctccgggaaggtgctggatagagagatcgcagggaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaagaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgca

SEQ ID NO: 50 - DNA, 2397 bp, synthetic construct
  source 1..2397: mol_type = other DNA
ggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggccgttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagtcaacgacgtgaagaagcaccgcggactggacatccaggcaaaaacagtggactccatcctgctaaacgggtgtcgtcgtgccgtggacatcctatatgtggacgaggctttcgcttgccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggtaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagaggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcagattcgacttggtctttgtgaacattcacacggaattcagaatccaccactaccagcagtgtgtcgaccacgccatgaagctgcagatgcttgggggagatgcgctacgactgctaaaacccggcggcagcctcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgt

SEQ ID NO: 51 - DNA, 1446 bp, synthetic construct
  source 1..1446: mol_type = other DNA
gcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagggcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccatagacatgaggacggctgtggagttgctcaatgatgacgtggagctgaccacagacttggtgagagtgcacccggacagcagcctggtgggtcgtaagggctacagtaccactgacgggtcgctgtactcgtacttggaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtggacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatcgacgacggaccactcagaccggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcatcctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgagactaggccgcgcgggtgca

SEQ ID NO: 52 - DNA, 1845 bp, synthetic construct
  source 1..1845: mol_type = other DNA
tatattttctcctcggacactggcagcggacatttacaacaaaaatccgttaggcagcacaatctccagtgcgcacaactggatgcggtcgaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggagagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctaaattggtgcgttaa

SEQ ID NO: 53 - DNA, 38 bp, synthetic construct
  source 1..38: mol_type = other DNA
tacacagaattctgattatagcgcactattatagcacc

SEQ ID NO: 54 - DNA, 261 bp, synthetic construct; identical to SEQ ID NO: 44
  source 1..261: mol_type = other DNA

SEQ ID NO: 55 - DNA, 162 bp, synthetic construct
  source 1..162: mol_type = other DNA
gttagggtaggcaatggcattgatatagcaagaaaattgaaaacagaaaaagttagggtaagcaatggcatataaccataactgtataacttataacaaagcgcaacaagacctgcgcaattggccccgtggtccgcctcacggaaactcggggcaactcat

SEQ ID NO: 56 - DNA, 102 bp, synthetic construct
  source 1..102: mol_type = other DNA
atggagttcatcccaacccaaactttttacaataggaggtaccagcctcgaccctggactccgcgctctactatccaaatcattaggcccagaccgcgccct

SEQ ID NO: 57 - DNA, 66 bp, synthetic construct; identical to SEQ ID NO: 47
  source 1..66: mol_type = other DNA

(19) United States
(12) Patent Application Publication
(10) Pub. No.: US 2026/0286321 A1
(43) Pub. Date: Sep. 24, 2026

(54) RNA REPLICONS, COMPOSITIONS AND METHODS OF USE THEREOF

(71) Applicant: ABLE SCIENCES, INC., Philadelphia, PA (US)

(72) Inventors: Jolly M. CHENDRIMADA, Philadelphia, PA (US); Thimmaiah CHENDRIMADA, Philadelphia, PA (US)

(73) Assignee: ABLE SCIENCES, INC., Philadelphia, PA (US)

(21) Appl. No.: 19/684,113

(22) Filed: May 21, 2026

Related U.S. Application Data
(63) Continuation-in-part of application No. PCT/US25/19232, filed on Mar. 10, 2025.
(60) Provisional application No. 63/563,108, filed on Mar. 8, 2024.

(51) Int. Cl. C12N 9/12; A61K 40/11; A61K 40/15; A61K 40/31; A61K 40/35; A61P 11/06; A61P 35/00; C07K 14/54; C07K 16/24; C07K 16/28; C12N 5/0783

(52) U.S. Cl. CPC C12N 9/127; A61K 40/11; A61K 40/15; A61K 40/31; A61K 40/35; A61P 11/06; A61P 35/00; C07K 14/5406; C07K 16/244; C07K 16/247; C07K 16/2803; C12N 5/0636; C12N 5/0646; C12Y 207/07048

(57) ABSTRACT

The present disclosure provides novel self-amplifying RNA (saRNA) constructs engineered for enhanced protein expression, prolonged durability, reduced immunogenicity, and homogeneous expression of multiple therapeutic proteins. The saRNA constructs comprise a 5′ untranslated region, non-structural protein genes derived from alphaviruses, at least one gene of interest encoding a therapeutic protein, a 3′ untranslated region, and modified nucleosides. Also disclosed are dual construct systems and multivalent polycistronic constructs with flexible, modular component arrangements, including bivalent through hexavalent embodiments encoding combinations of chimeric antigen receptors (CARs), cytokine-anchored chimeric antigen receptors (CCARs), cytokines, cytokine muteins, immune checkpoint inhibitors, and co-stimulatory molecules. Methods of producing and using the saRNA constructs for engineering immune cells for treatment of cancer, inflammatory conditions, and infectious diseases are provided.

### RELATED APPLICATIONS AND INCORPORATION BY REFERENCE

This application is a continuation-in-part of International Patent Application No. PCT/US25/19232, filed Mar. 10, 2025, published as WO 2025/189200 on Sep. 11, 2025, which claims priority to U.S. Provisional Patent Application No. 63/563,108, filed Mar. 8, 2024, the disclosures of which are incorporated herein by reference in their entirety.

The foregoing applications, and all documents cited therein or during their prosecution (“application cited documents”) and all documents cited or referenced in the applications cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

Embodiments described herein relate generally to novel saRNA replicons, compositions, and to methods for making and using the same. More specifically, the present disclosure relates to saRNA constructs engineered for enhanced protein expression, increased durability, and reduced immunogenicity, and to the use of such constructs to express chimeric antigen receptors (CARs), cytokine-anchored chimeric antigen receptors (CCARs), cytokines, and combinations thereof in immune cells for the treatment of cancer, eosinophilic diseases including severe eosinophilic asthma (SEA), hypereosinophilic syndrome (HES), chronic eosinophilic leukemia (CEL), and other inflammatory conditions, and other diseases.

### SEQUENCE LISTING

The instant application contains a Sequence Listing which has been submitted electronically in XML format and is hereby incorporated by reference in its entirety. Said XML copy, created on May 20, 2026, is named H8762-00007_SL.xml, and is 249,383 bytes in size.

### BACKGROUND OF THE INVENTION

Ribonucleic acid (RNA) is a polymeric molecule that is essential for most biological functions, either by performing the function itself (non-coding RNA) or by forming a template for the production of proteins (messenger RNA). RNA and deoxyribonucleic acid (DNA) are nucleic acids. The nucleic acids constitute one of the four major macromolecules essential for all known forms of life. RNA is assembled as a chain of nucleotides. Cellular organisms use messenger RNA (mRNA) to convey genetic information (using the nitrogenous bases of guanine, uracil, adenine, and cytosine, denoted by the letters G, U, A, and C) that directs synthesis of specific proteins. Many viruses encode their genetic information using an RNA genome. Over one hundred different nucleoside modifications have been identified in RNA (Rozenski et al., 1999, The RNA Modification Database: 1999 update. Nucl Acids Res 27:196-197). The extent and nature of modifications vary and depend on the RNA type as well as the evolutionary level of the organism from where the RNA is derived. Ribosomal RNA, the major constituent of cellular RNA, contains significantly more nucleoside modifications in mammalian cells than bacteria. Nucleoside modifications have a great impact on the immunostimulatory potential and on the translation efficiency of RNA.

The use of RNA technology to address therapeutic needs from immunological disorders, to cancer and numerous other medical conditions is widely recognized as having tremendous potential. While certain RNA-based therapeutics have been successful, problems still exist.

Citation or identification of any document in this application is not an admission that such document is available as prior art to the present invention.

For example, it is known that RNA may itself be associated with triggering an immune response. One solution to this problem is based on the recognition that the immunogenicity of RNA may be reduced by the incorporation of modified nucleosides with an associated increase in translation (Kariko et al., 2008, Mol Ther 16:1833-1840; Kariko et al., 2005, Immunity 23:165-175) potentially allowing efficient expression of proteins in-vivo and ex-vivo without activation of innate immune receptors. Though the concept of modified nucleosides has been developed, the array of such modifications is vast and there continues to be a need to identify specific modifications that are both effective and safe. There is also still a need for improved processes that are reliable and scalable for producing RNA constructs that are durable, amenable for off-the-shelf and autologous use, and that permit the incorporation of complex designs while simultaneously reducing immunogenicity.

Thus, there is a need in the art for efficient preparations of RNA replicons optimized for protein production, as well as improved immune cell engineering, decreased immunogenicity and durability.

### SUMMARY OF THE INVENTION

The use of RNA technology to address therapeutic needs from immunological disorders, to cancer and numerous other medical conditions is widely recognized as having tremendous potential. While certain RNA-based therapeutics have been successful, certain challenges remain.

Despite the promise of RNA-based therapeutics, existing RNA delivery approaches often suffer from short duration of expression, limited protein production, heterogeneous expression patterns, and immunogenicity concerns. These limitations have constrained the development of effective RNA-based cell engineering strategies, particularly for applications requiring sustained expression of therapeutic proteins or multi-protein expression. Conventional mRNA approaches typically provide protein expression for only 2-3 days, limiting their therapeutic utility in applications requiring longer-term expression. Additionally, when multiple mRNAs are delivered to a cell, the resulting protein expression is often heterogeneous, with individual cells expressing varying levels of each protein. The present disclosure addresses these limitations through novel saRNA constructs that provide enhanced protein expression, increased durability, reduced immunogenicity, and the ability to express multiple therapeutic proteins homogeneously.

Self-amplifying RNA (saRNA), derived from the genomes of alphaviruses such as Venezuelan equine encephalitis virus (VEEV), Semliki Forest virus (SFV), and Chikungunya virus (CHIKV), represents a potential solution to many of these limitations. The saRNA encodes non-structural proteins (NSP1-4) that form an RNA-dependent RNA polymerase complex capable of amplifying the RNA template, leading to enhanced and prolonged protein expression. However, wild-type saRNA constructs have their own limitations, including potential cytotoxicity due to viral origin of the RNA, sub-optimal expression in certain cell types (particularly primary human immune cells), and inherent design challenges in expressing multiple proteins homogeneously.

Furthermore, while permanent genetic integration methods (e.g., lentiviral or retroviral vectors) have been explored for engineering CAR-T cells, their use in chronic, non-malignant inflammatory conditions like asthma presents severe safety risks, including the potential for insertional mutagenesis and permanent on-target/off-tumor toxicity (such as irreversible eosinophil aplasia). The novel saRNA constructs of the present disclosure provide a highly optimized expression profile: the expression is sufficiently durable (persisting for weeks) to effectuate a profound, systemic depletion of pathogenic inflammatory cells and delay their regeneration, yet inherently transient, ensuring that the engineered cells eventually clear. This allows the host immune compartment to safely reconstitute over time, offering a substantially improved safety profile over permanent viral vector integration.

In some embodiments, the non-structural protein (NSP) genes comprise one or more point mutations in NSP1, NSP2, NSP3, NSP4, or a combination thereof.

In further embodiments, the non-structural protein genes comprise sequences derived from one or more viruses selected from the group consisting of alphaviruses, flaviviruses, coronaviruses, picornaviruses, rhabdoviruses, orthomyxoviruses, paramyxoviruses, bunyaviruses, arenaviruses, and retroviruses.

In certain implementations, the non-structural protein genes comprise sequences derived from one or more alphaviruses selected from the group consisting of EEV, CHIKV, SFV, EEEV, WEEV, RRV, SINV, MAYV, ONNV, BFV, MIDV, NDUV, GETV, AURAV, TROCV, WHAV, FMV, and HJV.

In particular embodiments, the saRNA construct is selected from: a) ABLE-003 (SEQ ID NO: 1); b) ABLE-004 (SEQ ID NO: 2); c) ABLE-005 (SEQ ID NO: 3); d) ABLE-006 (SEQ ID NO: 4); e) ABLE-007 (SEQ ID NO: 5); f) ABLE-008 (SEQ ID NO: 6); g) ABLE-009 (SEQ ID NO: 7); h) ABLE-010 (SEQ ID NO: 8); or i) ABLE-011 (SEQ ID NO: 9).

In one aspect, the saRNA constructs include modifications to enhance their performance in cell engineering applications.

In an embodiment, the gene of interest encodes a chimeric antigen receptor (CAR), a cytokine, an immune checkpoint inhibitor, an antibody, an antigen, or a combination thereof.

In another embodiment, the gene of interest encodes a therapeutic protein selected from an enzyme, hormone, antibody, transcription factor, or gene-editing enzyme.

In certain implementations, the 5′UTR and 3′UTR are modified for enhanced RNA transcription, translation and stability.

In some implementations, the saRNA construct further comprises modified nucleotides selected from pseudouridine, N1-methyl-pseudouridine, 5-methylcytidine, 5-hydroxymethylcytidine, 5-methyluridine, 2′-O-methylation; or a combination thereof.

In a further aspect, the construct comprises a polyadenylation sequence.

In one embodiment, the saRNA construct comprises a polycistronic sequence encoding two or more therapeutic proteins.

In a particular implementation, the polycistronic sequence encodes four therapeutic proteins.

In certain aspects, the two or more therapeutic proteins are expressed homogeneously.

In one aspect, the combined effect of these modifications may result in saRNA constructs with superior properties compared to wild-type saRNA or conventional mRNA, including enhanced protein expression, increased duration of expression, reduced immunogenicity, and the ability to express multiple therapeutic proteins simultaneously and homogeneously.

In particular embodiments, the saRNA constructs comprise sequences derived from different alphavirus species.

In one implementation, construct ABLE-007 (SEQ ID NO: 5), may include NSP2 from the CHIKV family, and constructs ABLE-010 (SEQ ID NO: 8) and ABLE-011 (SEQ ID NO: 9) may include NSP1-4 from the SFV family.

In an aspect, these chimeric constructs leverage the advantageous properties of different alphavirus replication machineries to optimize performance in specific cell types or applications.

In some embodiments, the saRNA constructs may also incorporate modified nucleotides to enhance stability and reduce immunogenicity.

In certain examples, such modifications include, but are not limited to, pseudouridine, N1-methyl-pseudouridine, 5-methylcytidine, 5-hydroxymethylcytidine, 5-methyluridine, and 2′-O-methylation.

In one aspect, these modifications may serve to evade cellular innate immune sensors, thereby reducing the immunogenicity of the RNA while enhancing its translation efficiency, stability and durability.

In certain aspects, provided herein is a pharmaceutical composition comprising: a) the RNA construct as described herein; and b) a delivery vehicle.

In some embodiments, the delivery vehicle comprises: a) lipid nanoparticles; b) polymeric nanoparticles; c) peptide-based carriers; d) viral vectors; e) exosomes; or f) combinations thereof.

In further implementations, the lipid nanoparticles comprise: a) an ionizable lipid; b) a helper lipid; c) cholesterol; d) a PEG-lipid; and/or e) an antibody for tissue targeted delivery.

In one aspect, provided herein is an isolated nucleic acid encoding the self-amplifying RNA construct as described herein.

In an embodiment, provided herein is a vector comprising the nucleic acid encoding the saRNA construct.

In particular implementations, provided herein is a plasmid comprising the nucleic acid encoding the saRNA construct.

In some aspects, provided herein are unique immune cells comprising novel saRNA replicons comprising single-stranded RNA as described above.

In one embodiment, provided herein is an engineered cell comprising the RNA construct as described herein.

In certain implementations, the cell is selected from BHK-21 cell lines, human primary immune cells including NK cells, Pan T cells, γδ T cells, B cells, dendritic cells, macrophages, monocytes, NKT cells, MAIT cells, CAR-T cells, memory T cells, regulatory B cells, myeloid-derived suppressor cells, induced pluripotent stem cells, mesenchymal stem cells, hematopoietic stem cells, or other stem cells.

In some embodiments, the engineered cell expresses one or more therapeutic proteins with homogeneous expression.

In a further aspect, the engineered cell expresses one or more therapeutic proteins for at least 14 days.

In an embodiment, provided herein is a population of engineered cells comprising the saRNA construct, wherein at least 80% of the cells express the encoded protein.

In an embodiment, the cells may be characterized as having decreased non-self RNA immunogenicity, increased protein expression and/or increased durability as compared to immune cells comprising reference RNA constructs (e.g., ABLE-002 SEQ ID NO: 11).

In an embodiment, the cells are engineered to attack one or more tumor-kill signals.

In an embodiment, cancer is a complex disease, one that may be characterized by a multitude of antigens, markers or kill signals.

In an embodiment, the novel saRNA replicons disclosed herein enable the engineering of unique immune cells having the ability to target one or more antigens, markers or tumor kill signals such that the resulting immune cells are configured to have an armored design, packed with multiple recombinant proteins capable of mounting a multi-pronged attack on a tumor cell.

In an embodiment, the immune cells comprising the novel saRNA replicons described herein may be engineered using various delivery methods, including but not limited to electroporation, lipid nanoparticle-mediated delivery, polymeric nanoparticle-mediated delivery, peptide-based carrier-mediated delivery, or viral vector-mediated delivery.

In one aspect, provided herein is a method of producing the saRNA construct, comprising: a) performing in vitro transcription; b) co-transcriptional capping; c) addition of a 3′UTR poly A tail; d) purifying the RNA construct; and e) formulating the RNA construct in a delivery vehicle.

In certain aspects, provided herein is a method of treating cancer comprising administering to a subject in need thereof: a) the pharmaceutical composition as described herein; or b) the engineered cell as described herein.

In some embodiments, the RNA construct encodes: a) a CAR targeting a tumor antigen; b) a cytokine; c) a checkpoint inhibitor; or d) combinations thereof.

In one aspect, provided herein is a method of treating an inflammatory condition comprising administering to a subject in need thereof: a) the pharmaceutical composition as described herein; or b) the engineered cells as described herein.

In another aspect, provided herein is a method of treating an infectious disease comprising administering to a subject in need thereof: a) the pharmaceutical composition as described herein; or b) the engineered cells as described herein.

In certain implementations, provided herein is a method of engineering immune cells comprising: a) isolating immune cells from a subject; b) introducing the saRNA construct as described herein into the immune cells; c) culturing the engineered cells; and d) confirming protein expression.

In some embodiments, provided herein is a method of in-vivo engineering of immune cells comprising introducing the LNP packaged saRNA as described herein into the body, wherein the LNP packed saRNA enters the immune cells, expresses the target, and engineers the cells.

In one implementation, introducing the RNA construct comprises: a) electroporation; b) lipofection; or c) viral transduction.

In certain aspects, the engineered immune cells may be administered to a subject in need thereof for the treatment of various conditions, including cancer, inflammatory conditions, and infectious diseases.

In an embodiment, the saRNA construct encodes a CRISPR-associated nuclease. In an embodiment, the saRNA construct encodes a CRISPR-associated nuclease and may include a guide RNA. In an embodiment, the saRNA construct may encode any component of a CRISPR system, including but not limited to Cas9, Cas12, Cas13, Cas3, base editors, prime editors, or other CRISPR-associated proteins or variants thereof. In an embodiment, the saRNA construct may further include one or more guide RNAs, scaffold RNAs, tracrRNAs, donor templates for homology-directed repair, regulatory elements that control expression of the CRISPR components, or combinations thereof. In an embodiment, the CRISPR system components may be engineered for enhanced specificity, reduced off-target effects, altered PAM requirements, or modified functional properties.

In certain implementations, provided herein is a method of gene editing comprising introducing into a cell the saRNA construct as described herein.

In some aspects, provided herein is a kit comprising: a) the self-amplifying RNA construct as described herein; b) a delivery vehicle; c) reagents for cell engineering; and d) instructions for use.

In addition to the single construct system, where both the non-structural protein genes and the gene(s) of interest are contained within a single saRNA molecule, in an embodiment, the present disclosure also encompasses a dual construct system.

In one aspect, provided herein is a dual construct self-amplifying RNA (saRNA) system comprising: a) a first construct comprising: i. a 5′ untranslated region (5′UTR); ii. non-structural protein genes; iii. a 3′ untranslated region (3′UTR); and iv. one or more modified nucleosides, modified nucleotides, modified internucleotide linkages, or combinations thereof; and b) a second construct comprising: i. a 5′ untranslated region (5′UTR); ii. at least one gene of interest encoding a therapeutic protein; iii. a 3′ untranslated region (3′UTR); and iv. one or more modified nucleosides, modified nucleotides, modified internucleotide linkages, or combinations thereof; v, wherein the first and second constructs are co-transduced for activity.

In an embodiment, the first construct comprises non-structural proteins selected from one or more of NSP1, NSP2, NSP3, and NSP4.

In some implementations, the second construct comprises a polycistronic sequence encoding two or more therapeutic proteins.

In the dual construct system, a first construct comprises the non-structural genes, while a second construct consists of one or more genes of interest.

In one aspect, the advantages of this approach include larger capacity for therapeutic gene payloads and improved manufacturing characteristics.

In some embodiments, both constructs are co-transduced for activity, with the non-structural proteins expressed from the first construct enabling amplification of the gene(s) of interest in the second construct.

In certain aspects, the dual construct system offers several advantages over traditional single-construct approaches.

In a first aspect, it allows for the inclusion of larger or multiple genes of interest, which may be limited by the packaging capacity of a single construct.

In a second aspect, it enables more flexible manufacturing processes, as the non-structural protein component and the gene(s) of interest component can be produced and optimized independently.

In a third aspect, it allows for greater control over the ratio of non-structural proteins to genes of interest, which can be optimized for specific applications or cell types.

In one embodiment, the saRNA constructs described herein function through a self-amplification mechanism.

In an implementation, upon delivery to a host cell, the saRNA is translated to produce the non-structural proteins (NSP1-4), which form an RNA-dependent RNA polymerase (RdRp) complex.

In a further implementation, this RdRp complex recognizes and binds to the subgenomic promoter present in the saRNA, generating negative-strand RNA intermediates, which then serve as templates for the production of multiple copies of the positive-strand RNA.

In certain aspects, the amplified RNA can be further translated to produce the encoded therapeutic proteins.

In one aspect, this self-amplification process results in the production of approximately 104 to 105 copies of the gene of interest from a single strand of saRNA, significantly enhancing protein expression levels compared to non-amplifying mRNA systems.

In some embodiments, the enhanced expression levels and extended duration of expression achieved with the saRNA constructs described herein make them particularly suited for applications requiring sustained therapeutic protein production, such as cancer immunotherapy.

In an embodiment, the unique immune cells comprising the novel saRNA constructs disclosed herein may be useful for therapeutic applications.

In an embodiment, the immune cells may be utilized to treat cancer or inflammatory conditions such as lupus, gout, psoriatic arthritis, myositis, scleroderma, rheumatoid arthritis, vasculitis, or Kawasaki Disease.

In an embodiment, the saRNA constructs described herein may be designed to express multiple therapeutic proteins in a polycistronic manner.

In an embodiment, a single saRNA construct may encode one or more chimeric antigen receptors (CARs) targeting different receptors (e.g., CD19-CAR and CD22-CAR) and a cytokine (e.g., IL-12 or IL-15), enabling a multi-pronged approach to cancer immunotherapy.

In an aspect, the polycistronic design of the saRNA constructs allows for homogeneous expression of multiple therapeutic proteins within the same cell, enhancing therapeutic efficacy compared to approaches relying on co-delivery of multiple separate mRNAs, which often result in heterogeneous expression patterns.

In an embodiment, the polycistronic saRNA constructs may express up to four therapeutic proteins from a single RNA molecule.

In one aspect, the homogeneous expression of multiple proteins enables the generation of “armored” immune cells capable of mounting a multi-pronged attack on target cells, such as tumor cells.

In a further aspect, this approach is particularly advantageous for addressing the heterogeneity and immune evasion mechanisms frequently observed in complex diseases like cancer.

In an embodiment, the present disclosure relates to novel saRNA replicons, compositions and methods for making and using the same. The novel constructs of the disclosure are characterized by having lower immunogenicity and higher durability in comparison to currently available constructs.

In certain aspects, the present disclosure addresses limitations through novel self-amplifying RNA (saRNA) constructs that provide enhanced protein expression, increased durability, reduced immunogenicity, and the ability to express multiple therapeutic proteins homogeneously.

In one aspect, provided herein is a self-amplifying RNA (saRNA) construct comprising: a) a 5′ untranslated region (5′UTR); b) non-structural protein genes; c) at least one gene of interest encoding a therapeutic protein; d) a 3′ untranslated region (3′UTR); and e) one or more modified nucleosides, modified nucleotides, modified internucleotide linkages, or combinations thereof.

In an aspect, provided herein are methods for genetic engineering of cells using the saRNA constructs described herein. The saRNA constructs can be utilized to introduce and express genes that modify cellular behavior, providing a framework for cell-based therapeutic strategies. These genetic engineering methods facilitate both transient and durable modifications, enabling a range of applications from vaccine development to adoptive cell therapy.

In some embodiments, the saRNA constructs described herein are delivered to cells ex-vivo, followed by administration of the genetically engineered cells to a subject. In other embodiments, the saRNA constructs are delivered directly to cells in-vivo, resulting in genetic modification within the subject's body.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF FIGURES

The present disclosure is best understood from the following detailed description when read in conjunction with the accompanying drawings. It is emphasized that, according to common practice, the various features of the drawings are not necessarily to scale. On the contrary, the dimensions of the various features are arbitrarily expanded or reduced for clarity. Like reference numerals denote like features throughout specification and drawings.

FIG. 1 provides VEEV replicons with modification in 5′UTR and NSPs for constructs ABLE-001 (SEQ ID NO: 10), ABLE-002 (SEQ ID NO: 11), ABLE-003 (SEQ ID NO: 1), ABLE-004 (SEQ ID NO: 2), ABLE-005 (SEQ ID NO: 3), and ABLE-006 (SEQ ID NO: 4).

FIG. 2 provides VEEV replicons including non-VEEV sequences for constructs ABLE-007 (SEQ ID NO: 5), ABLE-008 (SEQ ID NO: 6), ABLE-009 (SEQ ID NO: 7), ABLE-010 (SEQ ID NO: 8), and ABLE-011 (SEQ ID NO: 9).

FIG. 3 provides a listing of replicons having modified bases.

FIG. 4 provides the nucleotide sequence for Linear-ABLE-001-WT-VEEV-mCherry (SEQ ID NO: 10): wherein the 5′UTR region nucleotides comprises 1-44, the NSP1 region comprises nucleotides 45-1649, the NSP2 region comprises nucleotides 1650-4031, the NSP3 region comprises nucleotides 4032-5702, the NSP4 region comprises nucleotides 5703-7526, the SG promoter region comprises nucleotides 7527-7561, the mCherry region comprises nucleotides 7562-8272, the conserved VEEV sequence region comprises nucleotides 8273-8357, the 3′UTR region comprises nucleotides 8358-8712. FIG. 5 provides the nucleotide sequence for Linear-ABLE-002-TC83-VEEV-mCherry (SEQ ID NO: 11): wherein the 5′UTR region comprises nucleotides 1-44, the NSP1 region comprises nucleotides 45-1649, the NSP2 region comprises nucleotides 1650-4031, the NSP3 region comprises nucleotides 4032-5702, the NSP4 region comprises nucleotides 5703-7526, the SG promoter region comprises nucleotides 7527-7561, the mCherry region comprises nucleotides 7562-8272, the conserved VEEV sequence region comprises nucleotides 8273-8357, the 3′UTR region comprises nucleotides 8358-8712. FIG. 6 provides the nucleotide sequence for Linear-ABLE-003-TC-83-VEEV-g116u(NSP1)-E112Q(NSP2)-G31R(NSP3)-mCherry (SEQ ID NO: 1) wherein the 5′UTR region comprises nucleotides 1-44, the NSP1 region comprises nucleotides 45-1649, the NSP2 region comprises nucleotides 1650-4031, the NSP3 region comprises nucleotides 4032-5702, the NSP4 region comprises nucleotides 5703-7526, the SG promoter region comprises nucleotides 7527-7561, the mCherry region comprises nucleotides 7562-8272, the conserved VEEV sequence region comprises nucleotides 8273-8357, and the 3′UTR region comprises nucleotides 8358-8712.

FIG. 7 provides the nucleotide sequence for Linear-ABLE-004-TC-83-VEEV-g116u(NSP1)-mCherry (SEQ ID NO: 2) wherein the 5′UTR region comprises nucleotides 1-44, the NSP1 region comprises nucleotides 45-1649, the NSP2 region comprises nucleotides 1650-4031, the NSP3 region comprises nucleotides 4032-5702, the NSP4 region comprises nucleotides 5703-7526, the SG promoter region comprises nucleotides 7527-7561, the mCherry region comprises nucleotides 7562-8272, the Conserved VEEV sequence region comprises nucleotides 8273-8357, and the 3′UTR region comprises nucleotides 8358-8712.

FIG. 8 provides the nucleotide sequence for Linear-ABLE-005-TC-83-VEEV-g116u(NSP1)-E112Q(NSP2)-mCherry (SEQ ID NO: 3): wherein the 5′UTR region comprises nucleotides 1-44, the NSP1 region comprises nucleotides 45-1649, the NSP2 region comprises nucleotides 1650-4031, the NSP3 region comprises nucleotides 4032-5702, the NSP4 region comprises nucleotides 5703-7526, the SG promoter region comprises nucleotides 7527-7561, the mCherry region comprises nucleotides 7562-8272, the conserved VEEV sequence region comprises nucleotides 8273-8357, and the 3′UTR region comprises nucleotides 8358-8712.

FIG. 9 provides the nucleotide sequence for Linear-ABLE006-TC-83-VEEV-E112Q (NSP2)-G31R(NSP3)-mCherry (SEQ ID NO: 4): wherein the 5′UTR region comprises nucleotides 1-44, the NSP1 region comprises nucleotides 45-1649, the NSP2 region comprises nucleotides 1650-4031, the NSP3 region comprises nucleotides 4032-5702, the NSP4 region comprises nucleotides 5703-7526, the SG promoter region comprises nucleotides 7527-7561, the mCherry region comprises nucleotides 7562-8272, and the Conserved VEEV sequence region comprises nucleotides 8273-8357, and the 3′UTR region comprises nucleotides 8358-8712.

FIG. 10 provides the nucleotide sequence for Linear-ABLE-007-TC-83-VEEV-coCHIK V NSP2-mCherry (SEQ ID NO: 5): wherein the 5′UTR region comprises nucleotides 1-44, the NSP1 region comprises nucleotides 45-1649, the coCHIKV-NSP2 region comprises nucleotides 1650-4043, the NSP3 region comprises nucleotides 4044-5714, the NSP4 region comprises nucleotides 5715-7538, SG promoter region comprises nucleotides 7539-7573, the mCherry region comprises nucleotides 7573-8284, the Conserved VEEV sequence region comprises nucleotides 8285-8369, and the 3′UTR region comprises nucleotides 8370-8724.

FIG. 11 provides the nucleotide sequence for Linear-ABLE-008-TC-83-VEEV-5 prime and 51ntCSE-g116u(NSP1)-E112Q(NSP2)-G31R(NSP3)-mCherry (SEQ ID NO: 6): wherein the 5′UTR region comprises nucleotides 1-44, the NSP1 region comprises nucleotides 45-1649, the NSP2 region comprises nucleotides 1650-4031, the NSP3 region comprises nucleotides 4032-5702, the NSP4 region comprises nucleotides 5703-7526, the SG promoter region comprises nucleotides 7527-7535, the 5′UTR+51NTCSE region comprises nucleotides 7536-7738, the P2A region comprises nucleotides 7739-7804, the mCherry region comprises nucleotides 7805-8512, the conserved VEEV sequence region comprises nucleotides 8513-8597, and the 3′UTR region comprises nucleotides 8598-8952.

FIG. 12 provides the nucleotide sequence for Linear-ABLE-009-TC-83-VEEV-5 prime and 51ntCSE-g116u(NSP1)-E112Q(NSP2)G31R(NSP3)-3 prime UTR of SFV-mCherry (SEQ ID NO: 7): wherein the 5′UTR region comprises nucleotides 1-44, the NSP1 region comprises nucleotides 45-1649, the NSP2 region comprises nucleotides 1650-4031, the NSP3 region comprises nucleotides 4032-5702, the NSP4 region comprises nucleotides 5703-7526, the SG promoter region comprises nucleotides 7527-7535, the 5′UTR+51NTCSE region comprises nucleotides 7536-7738, the P2A region comprises nucleotides 7739-7804, the mCherry region comprises nucleotides 7805-8512, and the 3′UTR of SFV region comprises nucleotides 8513-8773.

FIG. 13 provides the nucleotide sequence for Linear-ABLE010-Semliki Forest Virus-WT-mCherry (SEQ ID NO: 8): wherein the 5′UTR region comprises nucleotides 1-85, the NSP1 region comprises nucleotides 86-1696, the NSP2 region comprises nucleotides 1697-4093, the NSP3 region comprises nucleotides 4094-5539, the NSP4 region comprises nucleotides 5540-7384, the SG promoter region comprises nucleotides 7385-7422, mCherry region comprises nucleotides 7423-8133, and the 3′UTR of SFV region comprises nucleotides 8134-8394.

FIG. 14 provides the nucleotide sequence for Linear-ABLE-011-Semliki Forest Virus—with enhancer-mCherry (SEQ ID NO: 9): wherein the 5′UTR region comprises nucleotides 1-85, the NSP1 region comprises nucleotides 86-1696, the NSP2 region comprises nucleotides 1697-4093, the NSP3 region comprises nucleotides 4094-5539, the NSP4 region comprises nucleotides 5540-7384, the SG promoter region comprises nucleotides 7385-7422, the Enhancer: region comprises nucleotides 7423-7524, the P2A: region comprises nucleotides 7525-7590, mCherry region comprises nucleotides 7591-8298, and the 3′UTR of SFV region comprises nucleotides 8299-8460.

FIG. 15 provides a schematic demonstrating the advantages of the novel saRNA constructs described herein.

FIG. 16 provides a schematic demonstrating the configuration of immune cells with multiple signals designed to support “armored designs” cells packed with multiple genes to mount a multi-pronged attack on tumors.

FIG. 17 provides a schematic showing the evolution of cell engineering approaches.

FIG. 18 provides a tumor kill assay using WT-NK cells, saRNA, or mRNA-generated CAR-NK cells.

FIG. 19 provides a flow chart demonstrating the optimization needed for application in ex-vivo and in-vivo therapeutics.

FIG. 20 provides a schematic illustrating the evolution of the Synthetic Optimized Long Acting RNA (SOLAR) technology, including the incorporation of point mutations, enhancer elements, and modified 3′UTRs.

FIG. 21 provides graphs demonstrating that SOLAR constructs elicit a significantly lower Type-1 Interferon response (IFN-α and IFN-β) in primary human pooled PBMCs compared to wild-type and TC-83 saRNA.

FIG. 22 provides fluorescence microscopy images and a bar graph demonstrating enhanced GFP expression and mRNA copy numbers in primary human Natural Killer (NK) cells using the SOLAR construct compared to unmodified constructs.

FIG. 23 provides fluorescence microscopy images and a bar graph comparing transfection efficiency, protein production, and RNA copy numbers between conventional mRNA and SOLAR in primary human NK cells.

FIG. 24 provides fluorescence microscopy images and an ELISA graph demonstrating the Quadruple Single-Shot™ multivalency of the SOLAR construct, showing simultaneous co-delivery and functional expression of four genes (mCherry, eGFP, and hIL-12 heterodimer) in primary human NK cells.

FIG. 25 provides a graphical model comparing the expression intensity and durability of mRNA, WT saRNA, TC-83 saRNA, and the SOLAR construct over time.

FIG. 26 provides fluorescence microscopy images showing effective expression of the SOLAR construct in both primary human Pan-T cells and primary human γδ-T cells.

FIG. 27 provides a line graph demonstrating durable and potent Raji-Luc2 tumor cell killing by SOLAR-CAR-T cells at Day 10 post-transfection.

FIG. 28 provides a schematic of the trivalent Severe Eosinophilic Asthma (SEA) product design, illustrating the simultaneous targeting of the IL-5, IL-4, and IL-13 pathways to achieve both short-term eosinophil depletion and long-term delay of regeneration.

FIG. 29 provides line graphs demonstrating durable killing of primary human eosinophils by IL-5-CCAR-T cells at Day 1 and Day 10 post-transfection.

FIG. 30 provides a schematic and a line graph demonstrating the recognition and killing of IL-5Rα positive EoL-1 human eosinophil cells by IL-5-CCAR-T cells.

FIG. 31 provides a schematic and a line graph demonstrating the recognition and killing of IL-5Rα expressing A549 cells by IL-5-CCAR-T cells.

FIG. 32 provides a line graph demonstrating the effective killing of IL-4 receptor positive Raji B-cells by IL-4-CCAR-T cells at Day 1 post-transfection.

FIG. 33 provides a line graph of a CD19-CAR T-cell tumor kill assay in Raji-Luc2 cells at Day 10 post-transfection.

FIG. 34 provides a line graph of an IL-4-CCAR T-cell assay demonstrating dose-dependent specific lysis of Raji-Luc2 cells at Day 1 post-transfection.

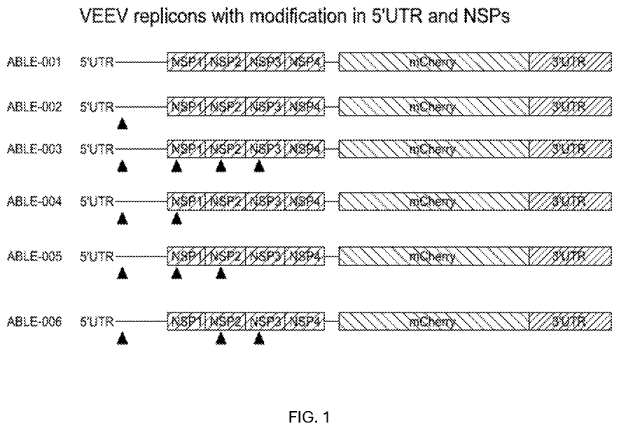

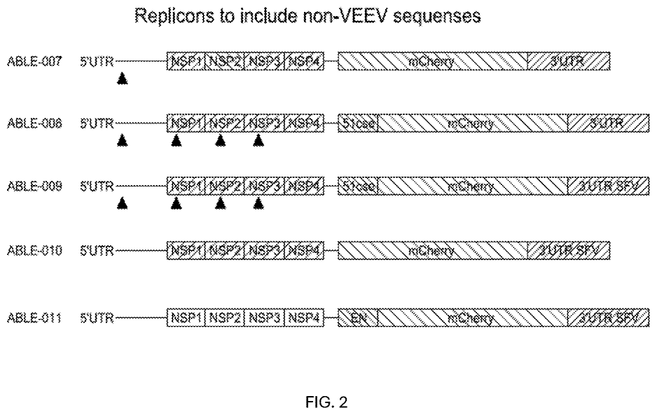

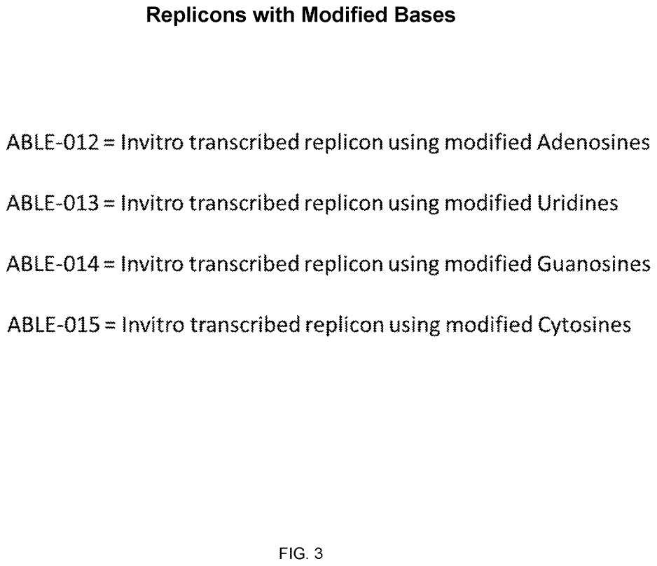

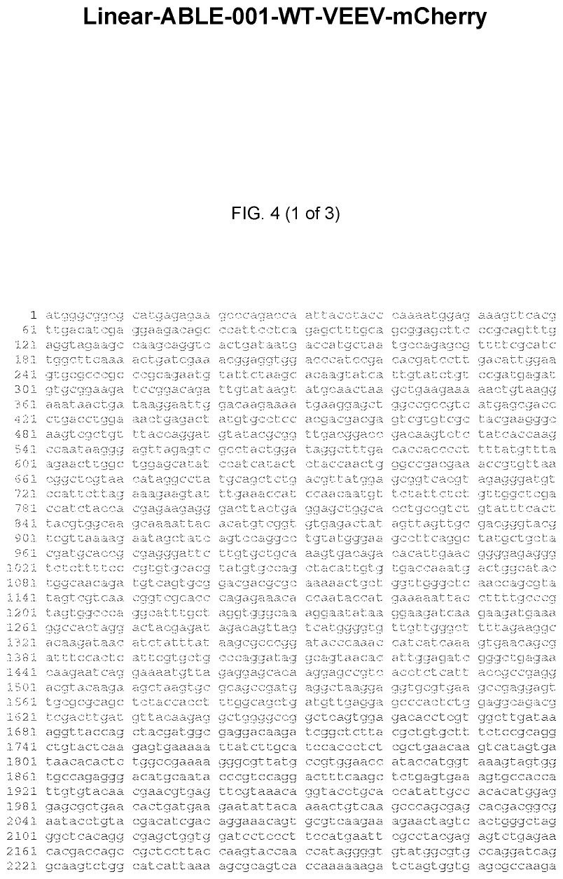

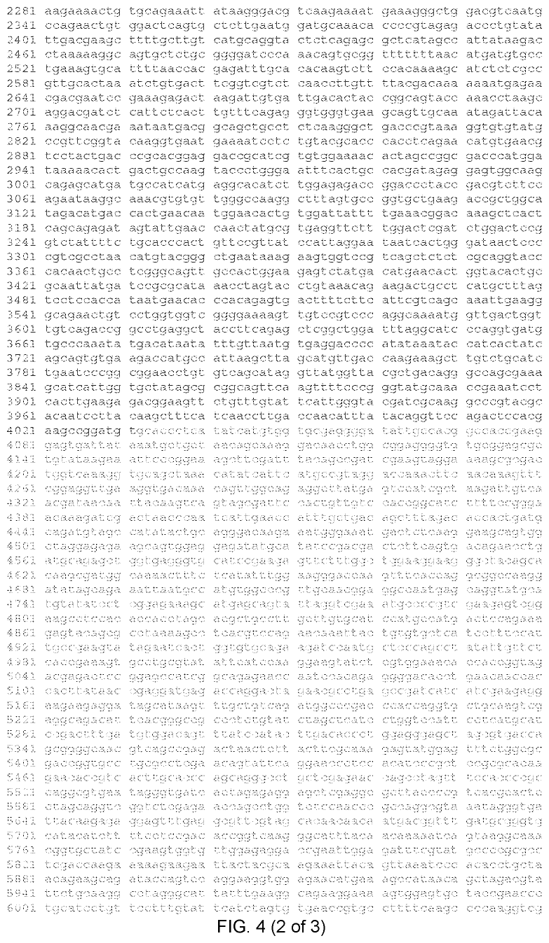

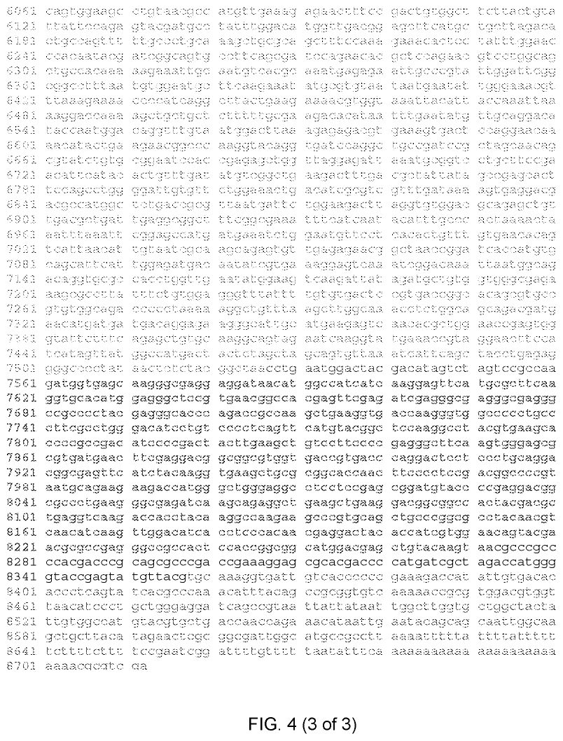

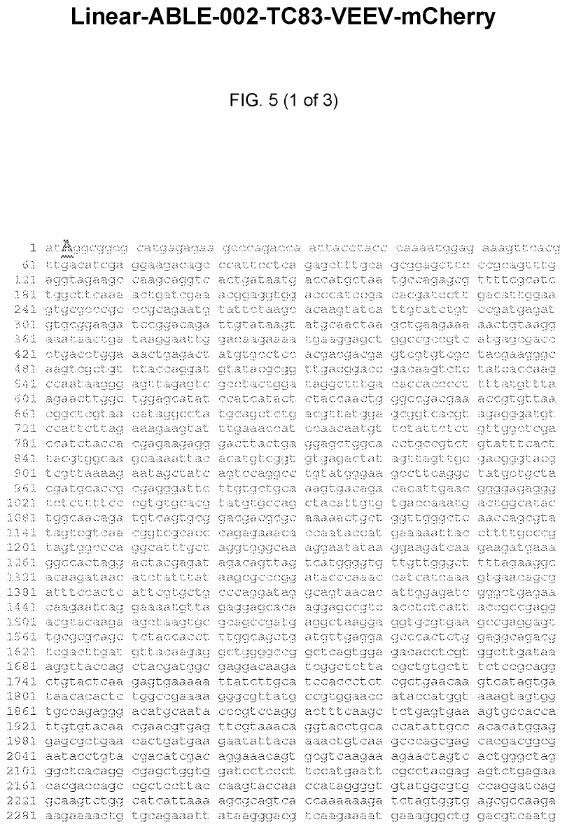

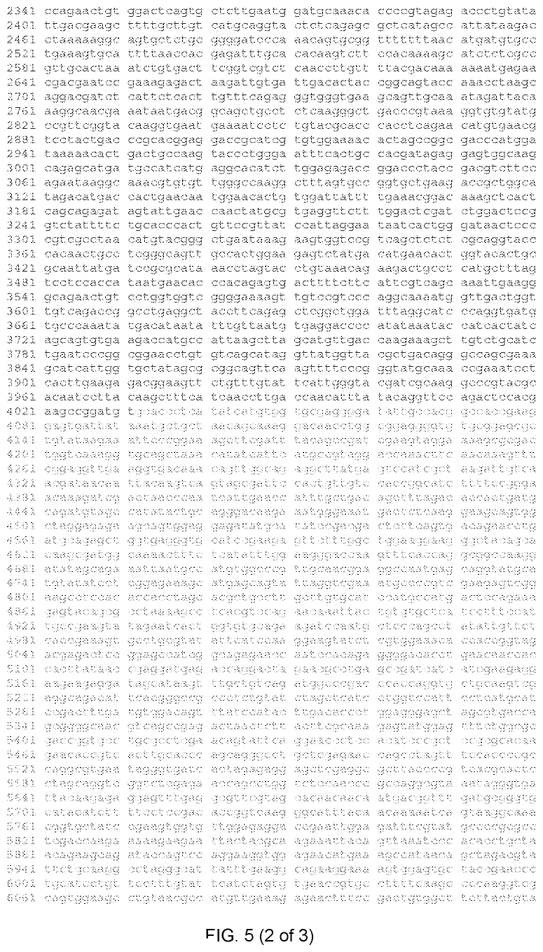

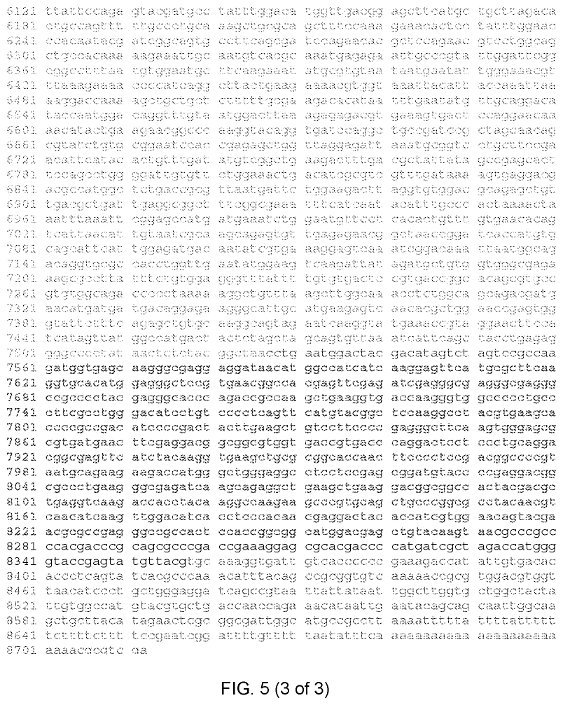

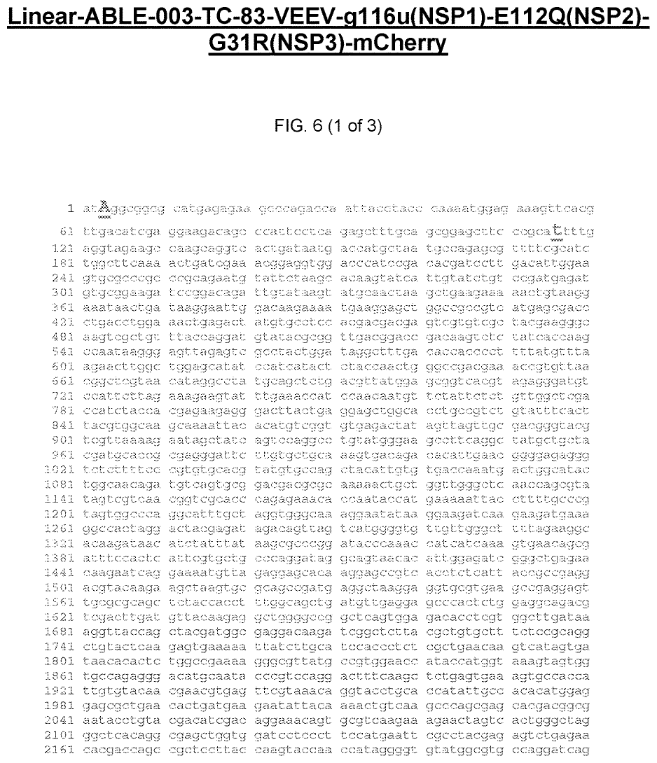

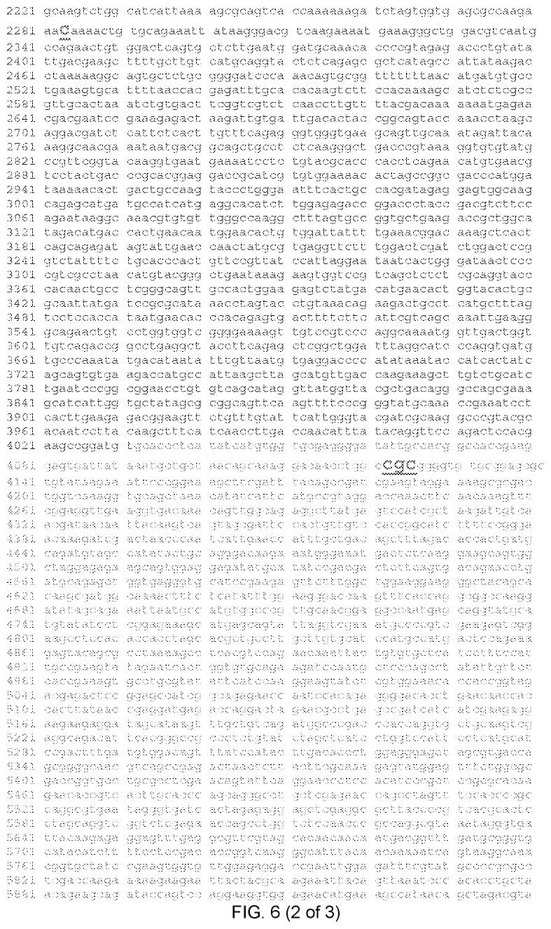

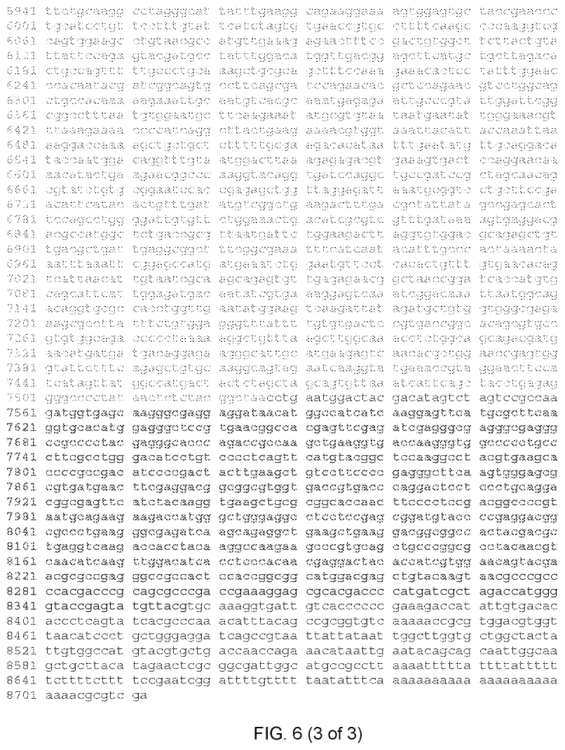

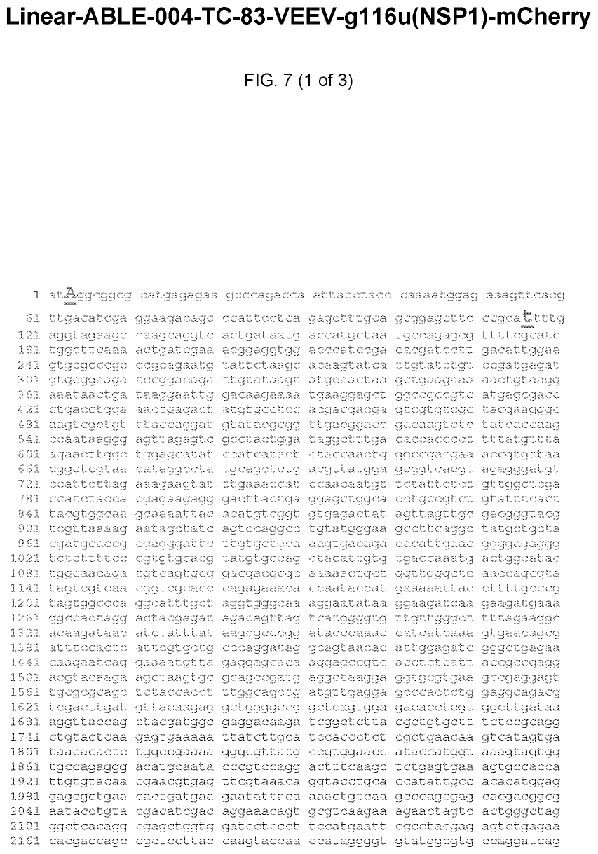

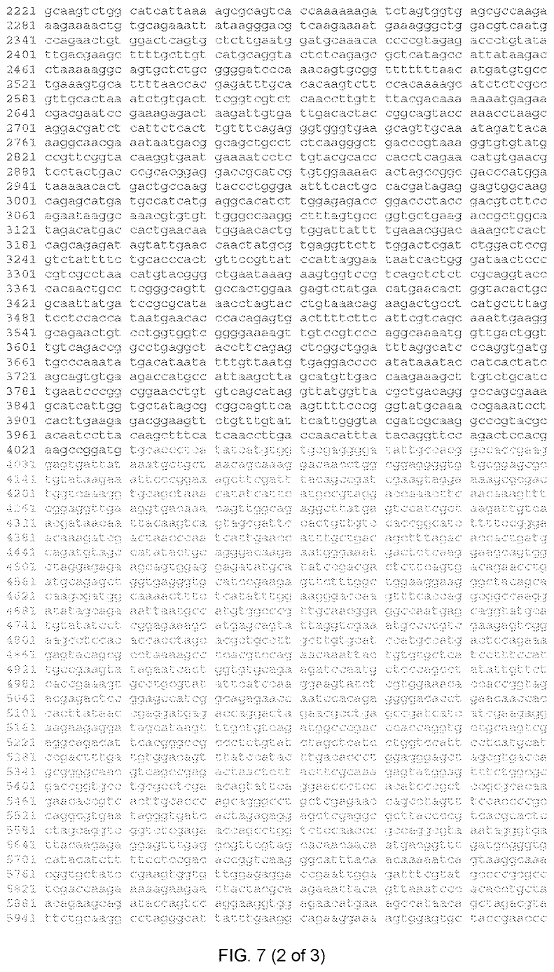

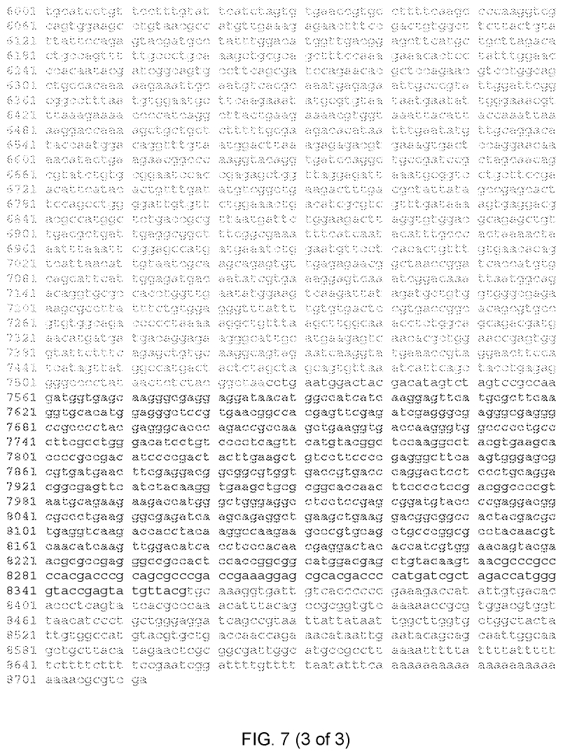

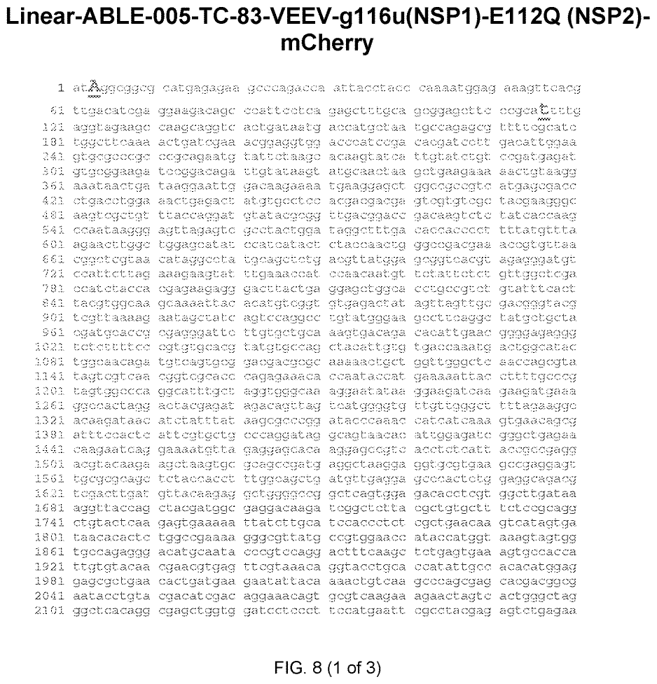

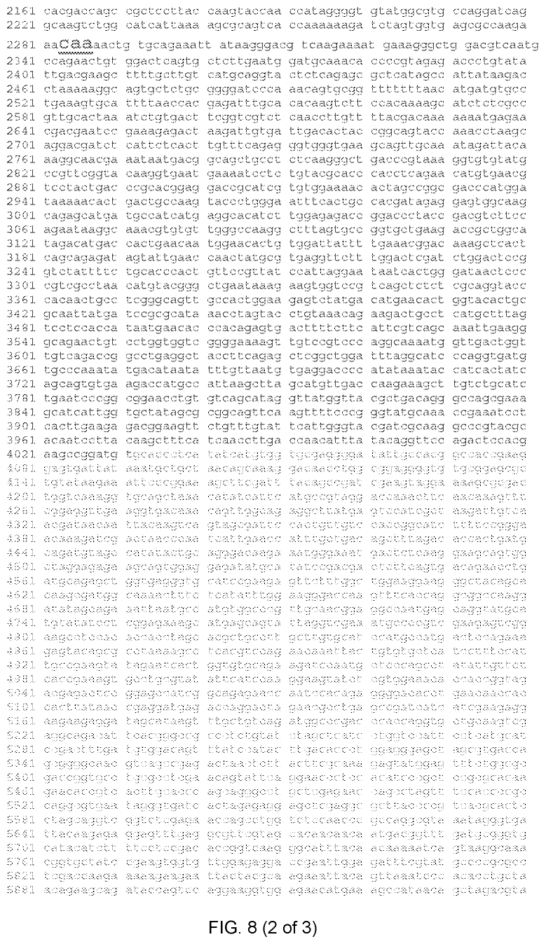

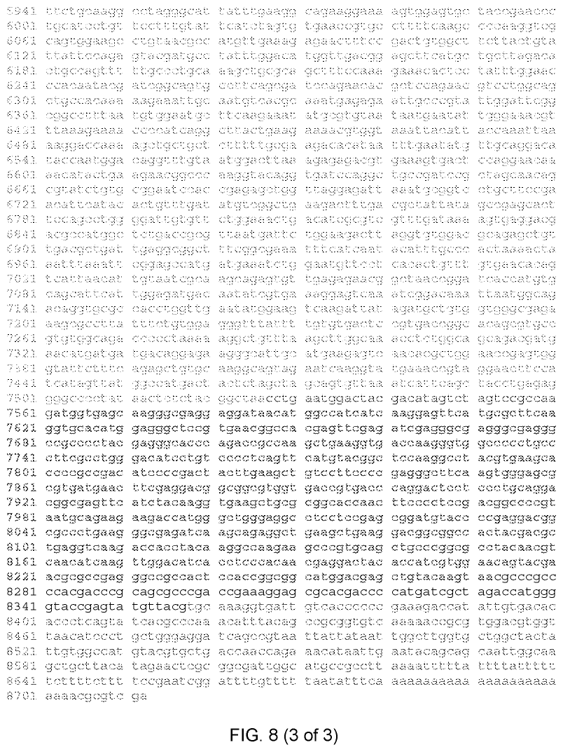

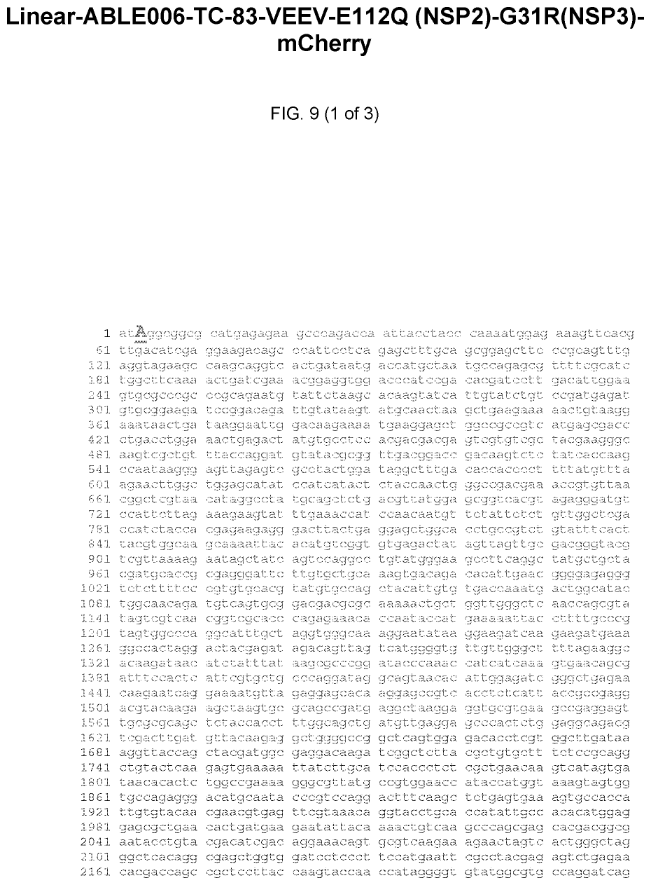

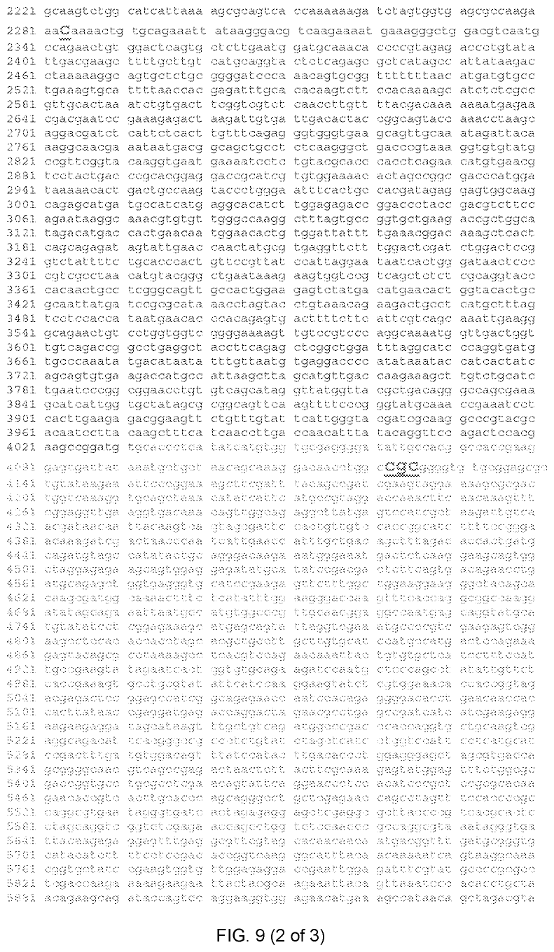

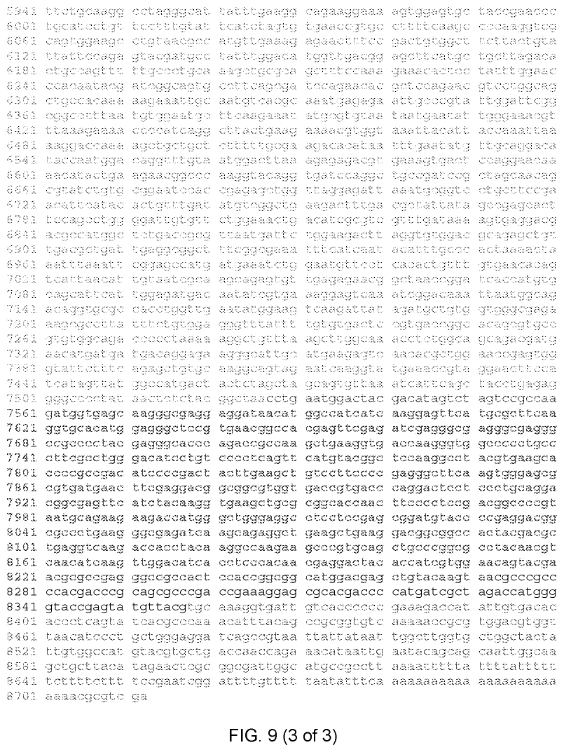

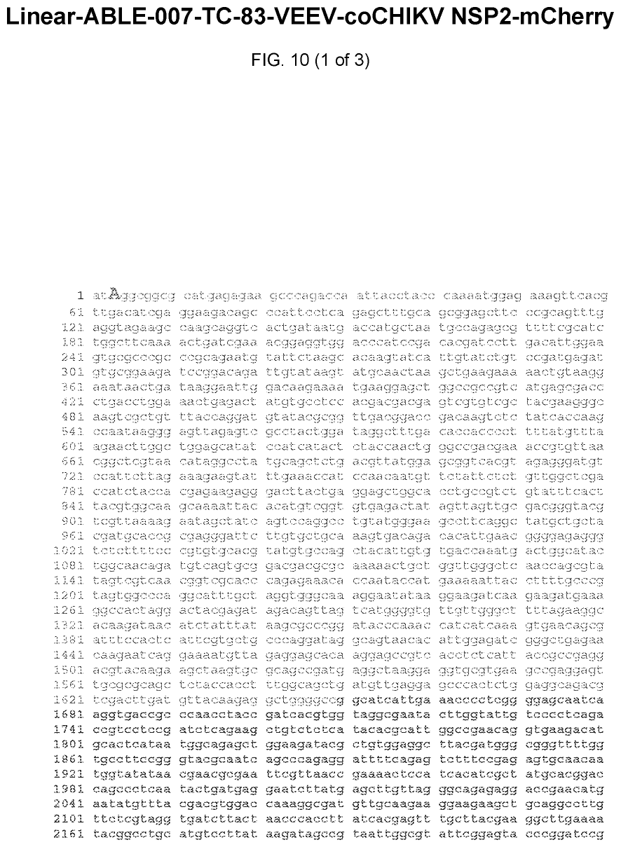

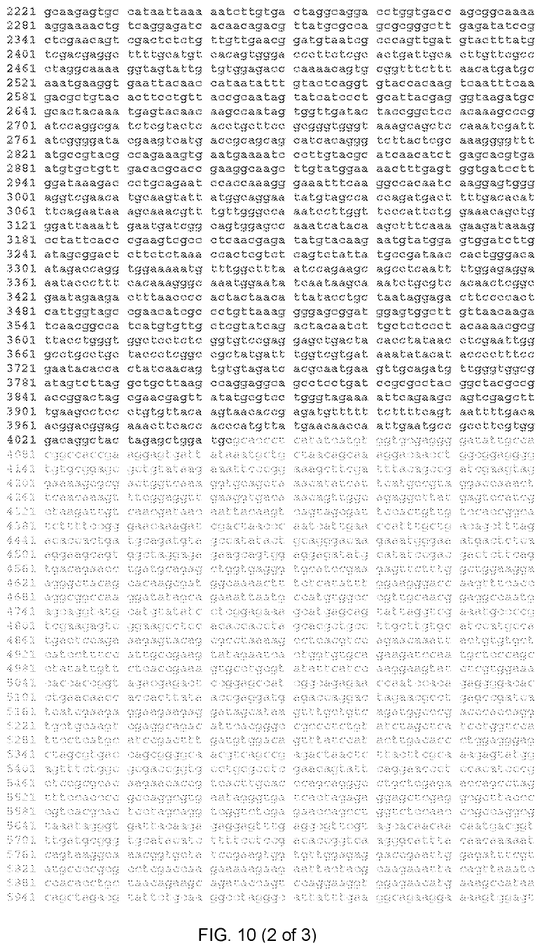

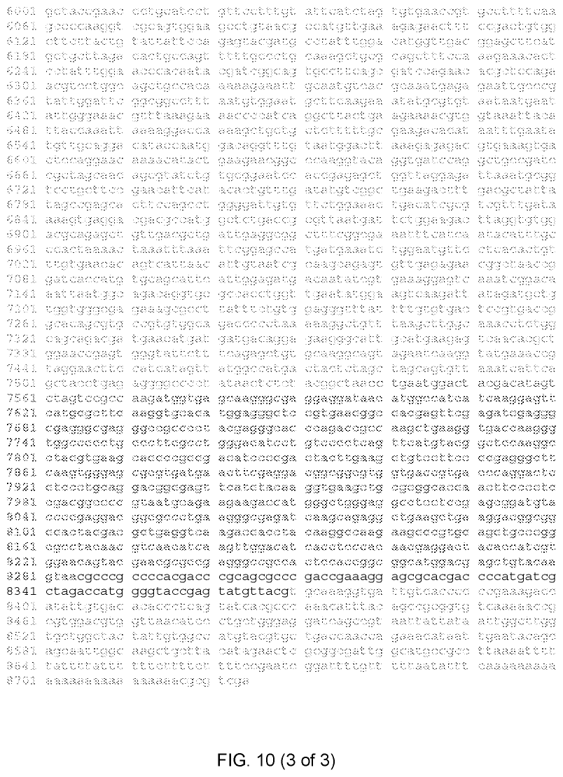

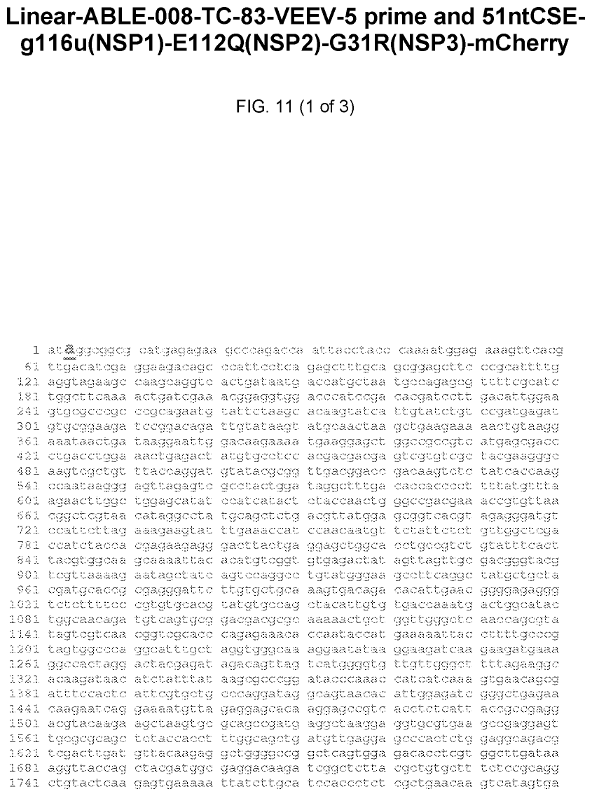

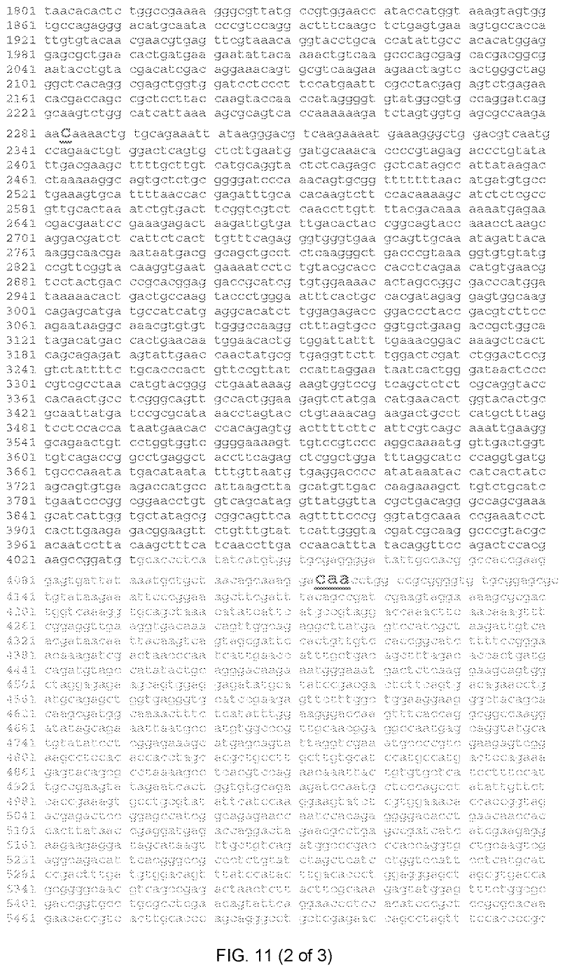

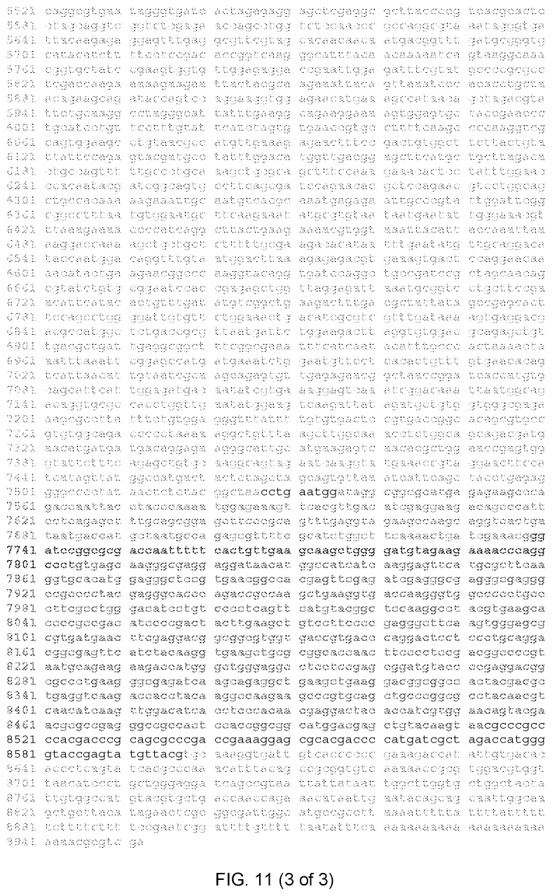

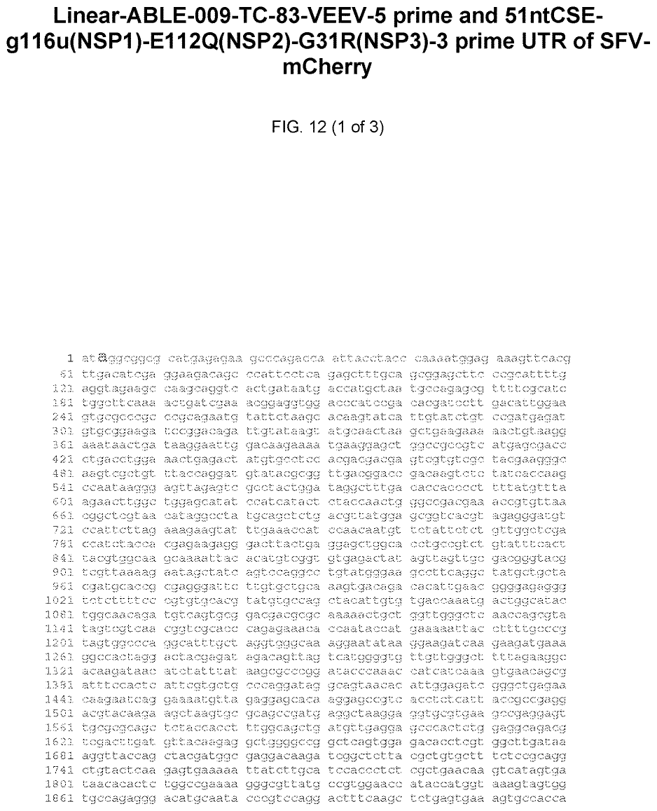

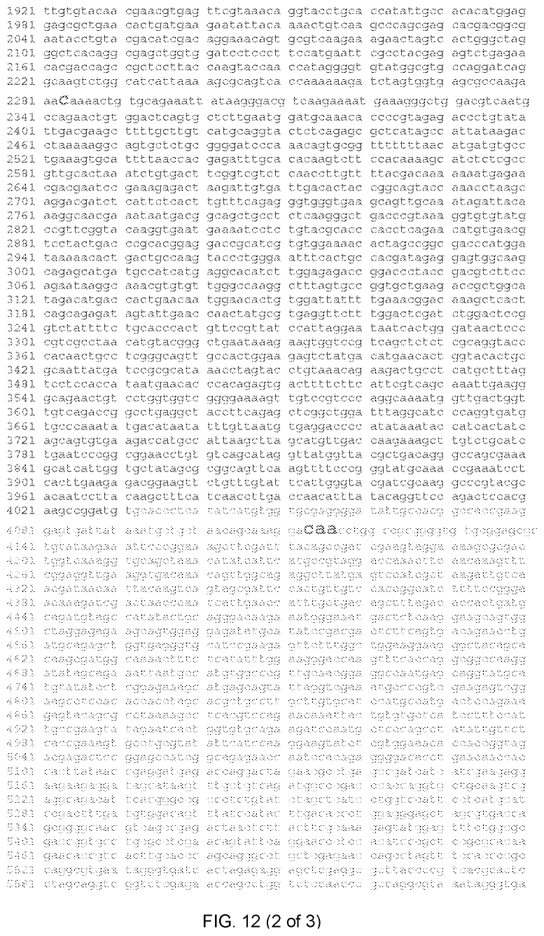

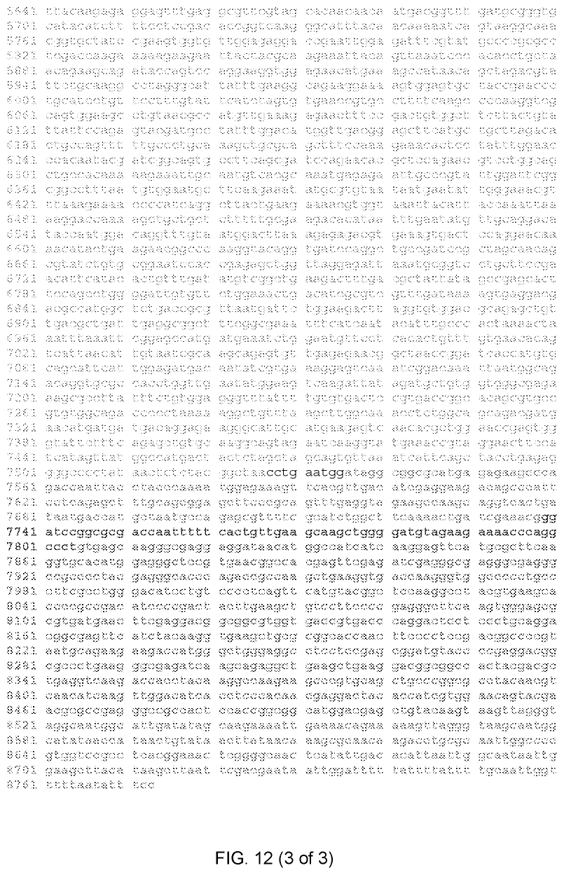

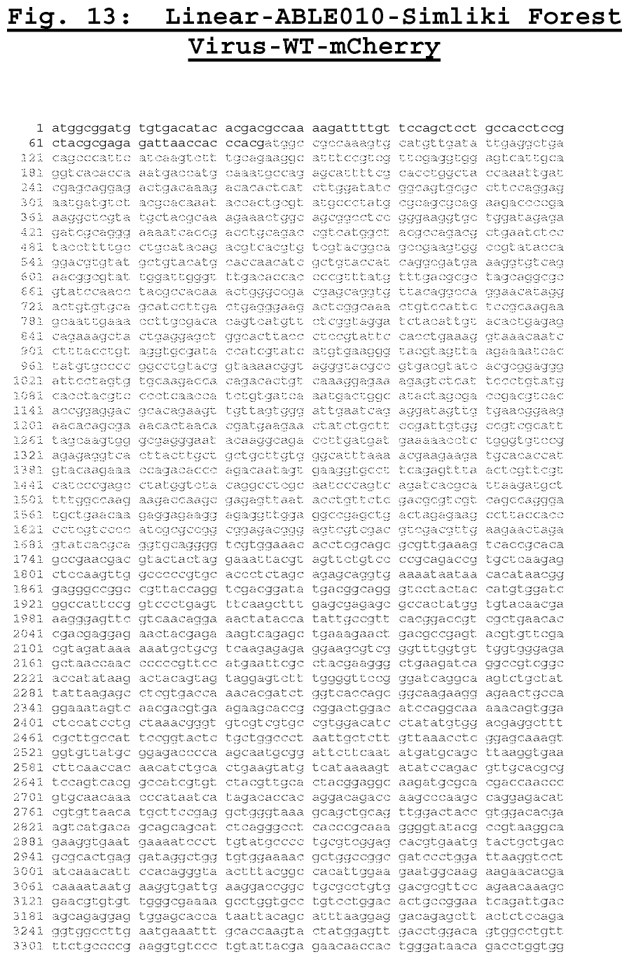

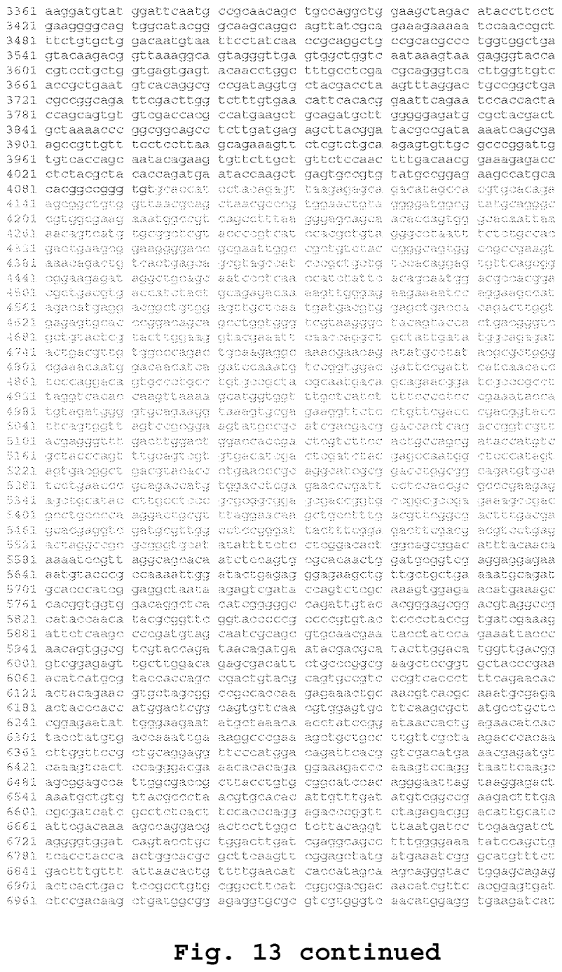

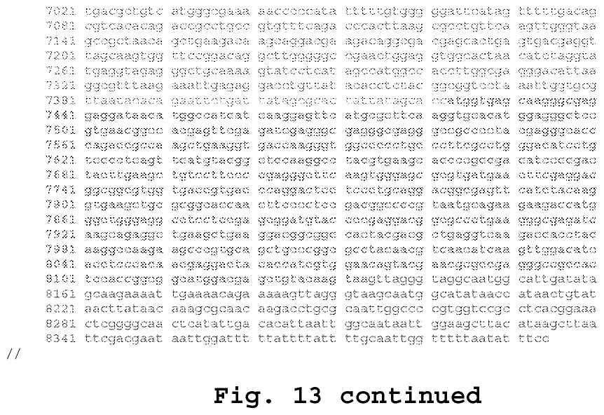

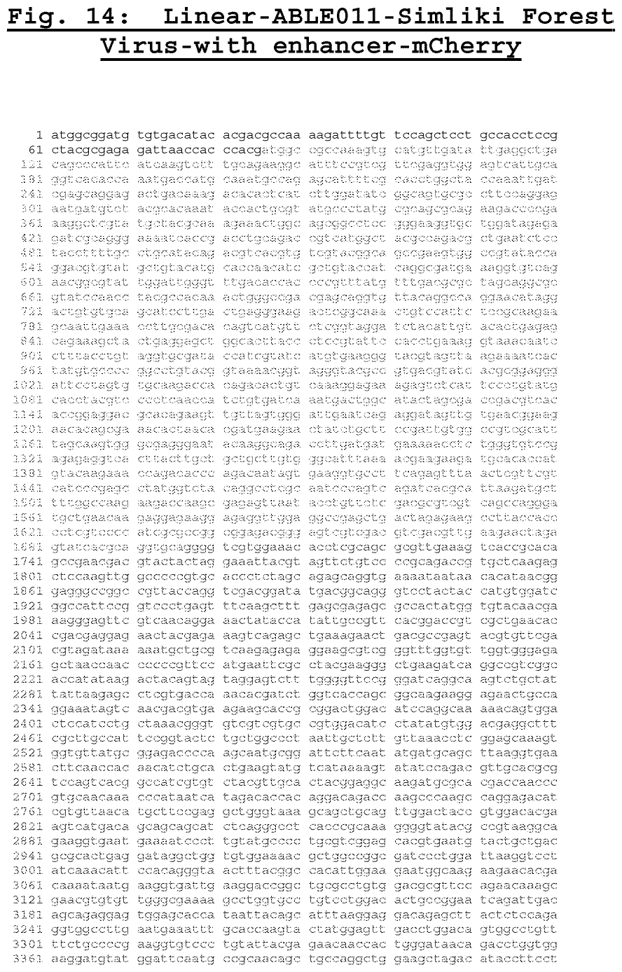

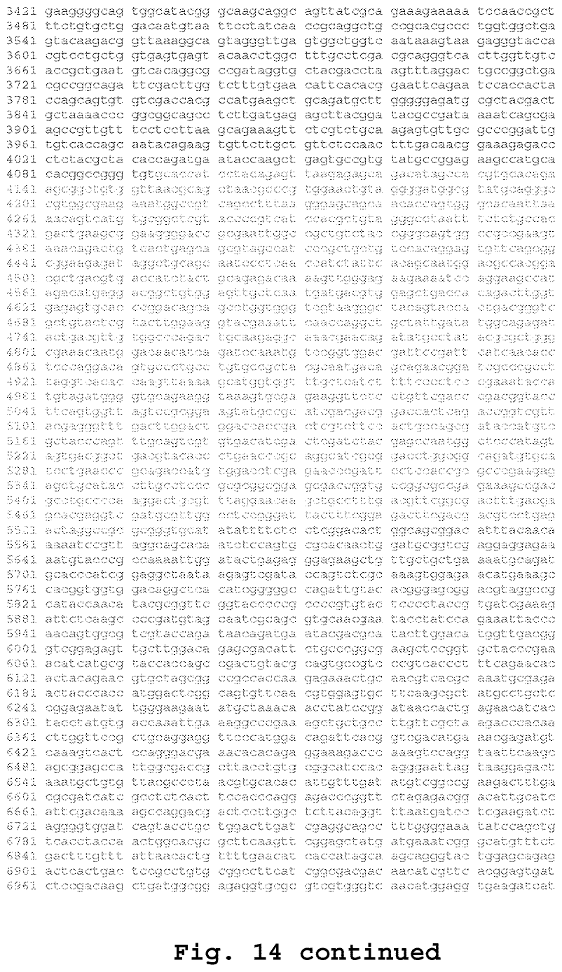

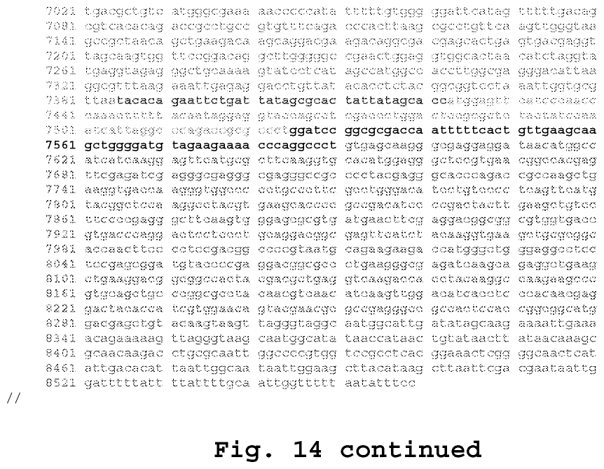

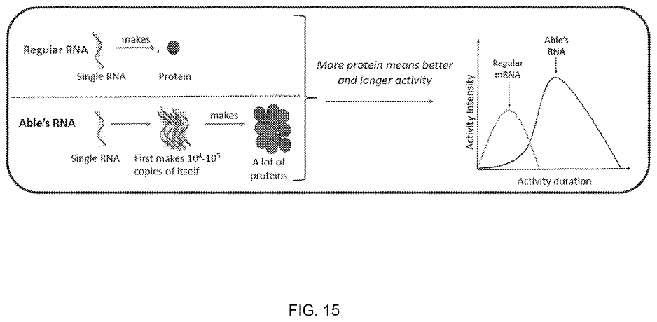

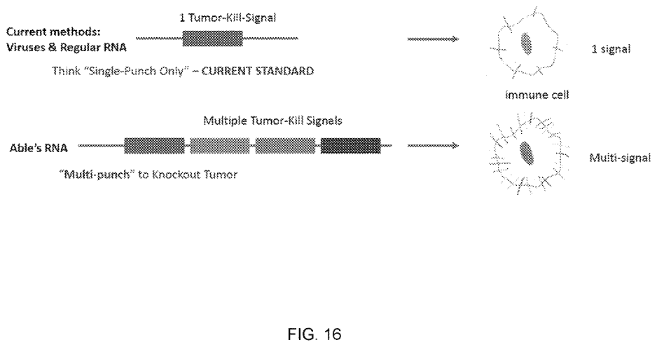

### DETAILED DESCRIPTION

The following detailed description is exemplary and explanatory and is intended to provide further explanation of the present disclosure described herein. Other advantages, and novel features will be readily apparent to those skilled in the art from the following detailed description of the present disclosure. Texts and references mentioned herein are incorporated in their entirety.

As used herein, the term “replicon” should be construed to mean disabled viral ssRNAs (single stranded RNAs) with autonomous RNA replication that drives high level, cytosolic expression of recombinant proteins.

As used herein, the term “saRNA” should be construed to mean “self-amplifying RNA.” In an aspect, an saRNA may be a Synthetic Optimized Long Acting RNA (SOLAR) construct engineered with point mutations, enhancer elements, and modified 3′ untranslated regions (3′UTRs) for enhanced stability and reduced immunogenicity.

As used herein, the term “non-structural protein” or “NSP” denotes the proteins encoded by the RNA replicon that facilitate RNA replication, wherein the term encompasses individual proteins (e.g., NSP1, NSP2, NSP3, NSP4) or combinations thereof, including any variants, fragments, or derivatives thereof.

As used herein, the term “modified nucleotide” means any nucleotide that has been chemically altered from its natural form, including but not limited to pseudouridine, N1-methyl-pseudouridine, 5-methylcytidine, and 2′-O-methylated nucleotides.

As used herein, the term “delivery vehicle” includes lipid nanoparticles, polymeric nanoparticles, peptide-based carriers, viral vectors, exosomes, or combinations thereof.

As used herein, the term “pharmaceutically acceptable carrier” refers to any material, composition, or vehicle that is compatible with biological systems and can be used to formulate the RNA replicon constructs for administration to a subject without causing significant adverse effects.

As used herein, the term “chimeric antigen receptor” or “CAR” refers to an engineered receptor protein that combines an extracellular antigen-binding domain with intracellular signalling domains capable of activating immune cells, particularly T cells or NK cells, to target specific antigens.

As used herein, the term “CCAR” refers to a Cytokine Chimeric Antigen Receptor, which comprises a specialized CAR-like receptor construct involving cytokine-related targeting, such as, for example, targeting the IL-4 or IL-5 pathways.

As used herein, the term “polycistronic sequence” refers to an RNA sequence that encodes multiple proteins from a single transcript, separated by elements such as internal ribosome entry sites (IRES) or 2A peptide sequences that enable the translation of each protein.

As used herein, the term “homogeneous expression” refers to the consistent and uniform expression of multiple proteins within the same cell or across a population of cells, as contrasted with heterogeneous expression where individual cells express varying levels of each protein.

As used herein, the term “durability” refers to the persistence of protein expression from the RNA construct over time, typically measured in days post-transfection or administration.

As used herein, the term “immunogenicity” refers to the ability of a substance, such as RNA, to provoke an immune response, with lower immunogenicity being generally desirable for therapeutic applications.

As used herein, the term “armored design” refers to immune cells engineered to express multiple therapeutic proteins that collectively enhance their anti-tumor or therapeutic efficacy through complementary mechanisms of action.

As used herein, the term “trivalent construct” refers to a polycistronic sequence or system such as an saRNA construct that simultaneously targets three distinct pathways, such as, for example, a construct encoding an IL-4 CCAR, an IL-5 CCAR, and a cytokine mutein (e.g., an IL-4 mutein) for the treatment of complex conditions, within a single RNA molecule. Also included within the scope of the invention are multivalent saRNA constructs wherein such constructs encode multiple sequences encoding multiple targets.

As used herein, “Severe Eosinophilic Asthma” or “SEA” refers to a phenotype of severe asthma characterized by elevated levels of eosinophils in the blood and airways, typically driven by Th2 inflammatory pathways including IL-4, IL-5, and IL-13, among others.

As used herein, the term “eosinophilic disease” includes, but is not limited to, severe eosinophilic asthma (SEA), hypereosinophilic syndrome (HES), chronic eosinophilic leukemia (CEL), eosinophilic esophagitis, eosinophilic chronic rhinosinusitis, and other conditions characterized by pathological eosinophil expansion or accumulation.

As used herein, the term “cytokine-anchored chimeric antigen receptor” or “CCAR” refers to a chimeric antigen receptor configuration that uses a cytokine (rather than a single-chain variable fragment (scFv)) as the extracellular target-binding domain, wherein the cytokine engages its cognate receptor on target cells to trigger T-cell-mediated or NK-cell-mediated killing in a receptor-dependent manner.

As used herein, the term “IL-4 mutein” refers to a variant of interleukin-4 that functions as an antagonist of IL-4 and IL-13 signaling by binding to the IL-4 receptor alpha (IL-4Rα) without inducing downstream signaling, thereby blocking the biological activity of both IL-4 and IL-13.

As used herein, the term “co-transduction” refers to the simultaneous delivery of multiple RNA constructs to the same cell, particularly in the context of the dual construct system described herein.

As used herein, the term “RNA-dependent RNA polymerase” or “RdRp” refers to an enzyme complex formed by the non-structural proteins that catalyzes the replication of RNA from an RNA template, enabling the self-amplification process of saRNA constructs.

As used herein, the term “Subgenomic promoter” refers to a sequence element within the saRNA construct that directs the expression of the gene(s) of interest. As used herein, the term “Untranslated region” or “UTR” refers to regions of the RNA construct that are not translated into protein but play important roles in regulating translation efficiency and RNA stability, including the 5′ untranslated region (5′UTR) and the 3′ untranslated region (3′UTR). In the present disclosure the singular forms “a,” “an,” and “the” include the plural reference, and reference to a particular numerical value includes at least that particular value, unless the context clearly indicates otherwise. Thus, for example, a reference to “a bead” or “a nano structure” is a reference to one or more of such structures and equivalents thereof known to those skilled in the art, and so forth. When values are expressed as approximations, by use of the antecedent “about,” it will be understood that the particular value forms another embodiment. As used herein, “about X” (where X is a numerical value) preferably refers to +10% of the recited value, inclusive. For example, the phrase “about 8” preferably refers to a value of 7.2 to 8.8, inclusive; as another example, the phrase “about 8%” preferably (but not always) refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such a listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” It is intended that any component, element, attribute, or step that is positively recited herein may be explicitly excluded in the claims, whether such components, elements, attributes, or steps are listed as alternatives or whether they are recited in isolation.

### RNA Replicons

In an embodiment, provided herein are novel saRNA replicons comprising single-stranded RNA identified as ABLE-003, ABLE-004, ABLE-005, ABLE-006, ABLE-007, ABLE-008, ABLE-009, ABLE-010, ABLE-011, and ABLE-012. In some embodiments, the single-stranded RNA (saRNA) encodes one or more proteins. In some embodiments, the saRNA is transient, safe, scalable, durable, autologous, suitable for complex designs.

In an embodiment, the saRNA constructs of the present disclosure include various modifications to enhance their performance in cell engineering applications. In an embodiment, these modifications may include, but are not limited to, alterations in the 5′UTR sequence, mutations in the non-structural protein genes (NSP1-4), incorporation of enhancer elements, inclusion of heterologous sequences from different alphavirus species, point mutations, addition of optimized 3′UTR sequences, and incorporation of modified nucleotides. In an embodiment, the combined effect of these modifications results in saRNA constructs with superior properties compared to wild-type saRNA or conventional mRNA, including enhanced protein expression, increased duration of expression, reduced immunogenicity, and the ability to express multiple therapeutic proteins simultaneously and homogeneously.

In an embodiment, the saRNA constructs comprise sequences derived from different alphavirus species. For example, in an embodiment, construct ABLE-007 includes NSP2 from the CHIKV family, and in an embodiment, constructs ABLE-010 and ABLE-011 include NSP1-4 from the SFV family. In an aspect, these chimeric constructs leverage the advantageous properties of different alphavirus replication machineries to optimize performance in specific cell types or applications.

In an embodiment, the saRNA constructs may also incorporate modified nucleotides to enhance stability and reduce immunogenicity. In an embodiment, examples of such modifications include, but are not limited to, pseudouridine, N1-methyl-pseudouridine, 5-methylcytidine, 5-hydroxymethylcytidine, 5-methyluridine, and 2′-O-methylation. In an aspect, these modifications serve to evade cellular innate immune sensors, thereby reducing the immunogenicity of the RNA while enhancing its translation efficiency and stability.

The unique combination of structural modifications within the Synthetic Optimized Long Acting RNA (SOLAR) constructs, including optimized 3′UTRs, specific point mutations within the replicase complex, and modified nucleosides, results in a synergistic and quantifiable reduction in non-self RNA immunogenicity. In primary human pooled peripheral blood mononuclear cells (PBMCs), the administration of the SOLAR constructs elicits a significantly attenuated Type-1 Interferon response compared to both wild-type saRNA and TC-83 saRNA. Specifically, the SOLAR constructs demonstrate a marked, statistically significant reduction in both Interferon-alpha (IFN-α) and Interferon-beta (IFN-β) secretion levels (measured in pg/ml) at 24 hours post-transfection. This suppressed reactogenicity profile allows for sustained, highly durable translation of the therapeutic payload by avoiding the premature cellular shutdown or apoptosis typically triggered by robust innate immune sensing of viral-derived RNA elements.

### Immune Cells Comprising RNA Replicons

Provided herein are unique immune cells comprising novel saRNA replicons comprising single-stranded RNA identified as, ABLE-003, ABLE-004, ABLE-005, ABLE-006, ABLE-007, ABLE-008, ABLE-009, ABLE-010, ABLE-011, and ABLE-012. The cells may be characterized as having decreased non-self RNA immunogenicity, increased protein expression and/or increased durability as compared to immune cells comprising ABLE-001 or ABLE-002. In an embodiment, the cells are engineered to attack one or more tumor-kill signals or inflammatory targets, such as eosinophils.

For example, it is known that cancer is a complex disease, one that may be characterized by a multitude of antigens, markers or kill signals. In an embodiment, these genetic markers, antigens, or kill signals targeted by the engineered immune cells may include, but are not limited to, CD19, CD20, CD22, BCMA (B-cell maturation antigen), CLEC12A, CD33, EGFRvIII, HER2, mesothelin, and PSMA for oncological applications. In an embodiment, the relevant genetic markers and cell-surface receptors include, but are not limited to, the Interleukin-5 receptor alpha subunit (IL-5Rα), the Interleukin-4 receptor alpha subunit (IL-4Rα), CCR3 (C—C chemokine receptor type 3), Siglec-8 (Sialic acid-binding Ig-like lectin 8), and EMRI (EGF-like module-containing mucin-like hormone receptor-like 1). The ability of the present saRNA platform to target these specific markers—either individually or simultaneously via polycistronic multivalent constructs—allows for precise cellular ablation of both malignant cells and aberrant inflammatory cell populations, such as pathogenic eosinophils and their progenitors. In an embodiment, the novel saRNA replicons disclosed herein enable the engineering of unique immune cells having the ability to target one or more antigens, markers, tumor kill signals such that the resulting immune cells are configured to have an armored design, packed with multiple recombinant proteins capable of mounting a multi-pronged attack on a tumor cell or targeted depletion of inflammatory cells and delay of their regeneration.

The immune cells comprising the novel saRNA replicons of the present disclosure may be engineered using various delivery methods, including but not limited to electroporation, lipid nanoparticle-mediated delivery, polymeric nanoparticle-mediated delivery, peptide-based carrier-mediated delivery, or viral vector-mediated delivery. The engineered immune cells may be administered to a subject in need thereof for the treatment of various conditions, including cancer, inflammatory conditions, and infectious diseases.

### Dual Construct System

In addition to the single construct system, where both the non-structural protein genes and the gene(s) of interest are contained within a single saRNA molecule, the present disclosure also encompasses a dual construct system. In the dual construct system, a first construct comprises the non-structural genes, while a second construct consists of one or more genes of interest. The advantage of this approach includes larger capacity for therapeutic gene payloads and improved manufacturing characteristics. Both constructs are co-transduced for activity, with the non-structural proteins expressed from the first construct enabling amplification of the gene(s) of interest in the second construct.

In an embodiment, the dual construct system offers several advantages over traditional single-construct approaches. First, it allows for the inclusion of larger or multiple genes of interest, which may be limited by the packaging capacity of a single construct. Second, it enables more flexible manufacturing processes, as the non-structural protein component and the gene(s) of interest component can be produced and optimized independently. Third, it allows for greater control over the ratio of non-structural proteins to genes of interest, which can be optimized for specific applications or cell types.

### Self-Amplification Mechanism

The saRNA constructs described herein function through a self-amplification mechanism. Upon delivery to a host cell, the saRNA is translated to produce the non-structural proteins (NSP1-4), which form an RNA-dependent RNA polymerase (RdRp) complex. This RdRp complex recognizes and binds to the saRNA, generating negative-strand RNA intermediates, which then serve as templates for the production of multiple copies of the positive-strand RNA. The amplified RNA can be further translated to produce the encoded therapeutic proteins.

This self-amplification process results in the production of approximately 104 to 105 copies of the gene of interest from a single strand of saRNA, significantly enhancing protein expression levels compared to non-amplifying mRNA systems. The enhanced expression levels and extended duration of expression achieved with the saRNA constructs described herein make them particularly suited for applications requiring sustained therapeutic protein production, such as cancer immunotherapy and durable treatment of chronic inflammatory conditions.

Beyond the extended duration of expression, the optimized self-amplification mechanism of the SOLAR constructs yields a substantially higher Protein-to-RNA ratio than conventional linear mRNA. When delivered at equivalent RNA dosing levels (e.g., 1 μg) and equivalent transfection efficiencies, the SOLAR constructs drive significantly greater intracellular accumulation of both the target mRNA transcripts and the resulting therapeutic proteins. In primary human immune cells, such as Natural Killer (NK) cells, the SOLAR constructs have demonstrated an excess of a 60-fold enhancement in target mRNA expression relative to conventional, non-amplifying mRNA controls. This superior translation efficiency allows for the use of lower administered doses of the saRNA payload or LNP formulation, thereby minimizing potential dose-limiting toxicities while achieving highly potent and sustained cellular engineering.

### Polycistronic Design

In an embodiment, the saRNA constructs of the present disclosure may be designed to express multiple therapeutic proteins in a polycistronic manner. For example, a single saRNA construct may encode one or more chimeric antigen receptors (CARs) or cytokine chimeric antigen receptors (CCARs) targeting different antigens (such as CD19-CAR and CD22-CAR) and a cytokine or cytokine mutein (such as IL-12, IL-15, or an IL-4 mutein), enabling a multi-pronged approach to cancer immunotherapy or the treatment of eosinophilic conditions. The polycistronic design of the saRNA constructs allows for homogeneous expression of multiple therapeutic proteins within the same cell, enhancing therapeutic efficacy compared to approaches relying on co-delivery of multiple separate mRNAs, which often result in heterogeneous expression patterns.

In an embodiment, the polycistronic saRNA constructs may express up to four (or more) therapeutic proteins from a single RNA molecule, such as, for example a trivalent construct encoding an IL-4 CCAR, an IL-5 CCAR, and an IL-4 mutein. The homogeneous expression of multiple proteins enables the generation of “armored” immune cells capable of mounting a multi-pronged attack on target cells, such as tumor cells or aberrant inflammatory cells (e.g., eosinophils in SEA). This approach is particularly advantageous for addressing the heterogeneity and immune evasion mechanisms frequently observed in complex diseases like cancer and therapy-resistant respiratory conditions.

In an embodiment, the saRNA constructs of the present disclosure may be designed to express multiple therapeutic proteins in a polycistronic manner (multivalent constructs). For example, a single saRNA construct may encode one or more chimeric antigen receptors (CARs) or cytokine chimeric antigen receptors (CCARs) targeting different antigens (e.g., CD19-CAR and CD22-CAR) and a cytokine or cytokine mutein (e.g., IL-12, IL-15, or an IL-4 mutein), enabling a multi-pronged approach to cancer immunotherapy or the treatment of eosinophilic conditions. The polycistronic design of the saRNA constructs allows for homogeneous expression of multiple therapeutic proteins within the same cell, enhancing therapeutic efficacy compared to approaches relying on co-delivery of multiple separate mRNAs, which often result in heterogeneous expression patterns.

In an embodiment, a trivalent construct comprises a first cytokine-anchored chimeric antigen receptor (CCAR), a second CCAR, and a cytokine or cytokine mutein. The arrangement of these components within the polycistronic sequence is flexible and not limited to a fixed order. For example, in an embodiment, the arrangement of the components can be [first CCAR]-[cytokine or cytokine mutein]-[second CCAR], or [second CCAR]-[cytokine or cytokine mutein]-[first CCAR], or [cytokine or cytokine mutein]-[first CCAR]-[second CCAR], or any other permutation. The components may be operably linked by elements such as internal ribosome entry sites (IRES) or 2A self-cleaving peptide sequences to ensure the expression of each therapeutic protein from the single saRNA molecule.

In an embodiment, the polycistronic saRNA constructs may express up to three or four therapeutic proteins from a single RNA molecule, such as, for example, a trivalent construct encoding an IL-4 CCAR, an IL-5 CCAR, and an IL-4 mutein. The homogeneous expression of multiple proteins enables the generation of “armored” immune cells capable of mounting a multi-pronged attack on target cells, such as tumor cells or aberrant inflammatory cells (e.g., eosinophils in SEA). In an embodiment, the polycistronic saRNA constructs of the present disclosure demonstrate “Quadruple Single-Shot” multivalency, enabling the simultaneous co-delivery and functional expression of four distinct gene sequences from a single RNA transcript. This high-capacity armored design is particularly advantageous for the expression of complex, multi-subunit therapeutic proteins alongside targeting receptors. For example, a quadruple saRNA construct may encode two distinct CAR or CCAR targeting moieties, alongside the separate subunits of a heterodimeric cytokine (e.g., the P40 and P35 subunits of human IL-12) separated by IRES or 2A cleavage elements. This enables the localized, stoichiometric secretion of fully assembled, functional heterodimeric cytokines from the engineered cell, significantly amplifying the therapeutic potency of the multi-valent immune cell without the need for multiple independent transfections. This approach is particularly advantageous for addressing the heterogeneity and immune evasion mechanisms frequently observed in complex diseases like cancer and therapy-resistant respiratory conditions.

In an embodiment, provided herein are novel saRNA replicons comprising single-stranded RNA identified as ABLE-003 (SEQ ID NO: 1), ABLE-004 (SEQ ID NO: 2), ABLE-005 (SEQ ID NO: 3), ABLE-006 (SEQ ID NO: 4), ABLE-007 (SEQ ID NO: 5), ABLE-008 (SEQ ID NO: 6), ABLE-009 (SEQ ID NO: 7), ABLE-010 (SEQ ID NO: 8), and ABLE-011 (SEQ ID NO: 9), as well as saRNA sequences encoding multivalent therapeutic payloads (e.g., SEQ ID NO: 10, SEQ ID NO: 11, SEQ ID NO: 12), where these saRNA replicons have distinct advantages over previous RNA technologies, including enhanced protein expression, increased duration of expression, and reduced immunogenicity.

In an embodiment, the saRNA constructs described herein incorporate modified nucleotides to enhance stability and reduce immunogenicity, with modifications including, but not limited to, pseudouridine, N1-methyl-pseudouridine, 5-methylcytidine, 5-hydroxymethylcytidine, 5-methyluridine, and 2′-O-methylation.

In an embodiment, the saRNA constructs described herein comprise sequence elements derived from different alphavirus species, resulting in chimeric constructs with optimized replication and expression properties in specific cell types.

In an embodiment, immune cells comprising the novel saRNA replicons described herein exhibit decreased non-self RNA immunogenicity, increased protein expression, and increased durability compared to immune cells comprising previous RNA replicon technologies.

In an embodiment, the unique immune cells comprising the novel saRNA replicons described herein are engineered to attack one or more tumor-kill signals or cytokine-mediated pathways, where the cells are configured to have an armored design packed with multiple recombinant proteins capable of mounting a multi-pronged attack on a tumor cell or target inflammatory cell (e.g., eosinophils).

In an embodiment, the saRNA constructs described herein are designed to express multiple therapeutic proteins in a polycistronic manner, allowing for homogeneous expression of up to three or four (or more) therapeutic proteins from a single RNA molecule, such as a trivalent IL-4 CCAR/IL-5 CCAR/IL-4 mutein payload.

In an embodiment, the dual construct system described herein, comprising a first construct with non-structural genes and a second construct with one or more genes of interest, provides advantages including larger capacity for therapeutic gene payloads and improved manufacturing characteristics.

In an embodiment, the self-amplification process of the saRNA constructs described herein results in the production of approximately 104 to 105 copies of the gene of interest from a single strand of saRNA, significantly enhancing protein expression levels compared to non-amplifying mRNA systems.

In an embodiment, the unique immune cells comprising the novel saRNA replicons described herein are useful for therapeutic applications, including the treatment of cancer and inflammatory conditions, such as severe eosinophilic asthma (SEA).

In an embodiment, the immune cells comprising the novel saRNA replicons described herein may be engineered using various delivery methods, including but not limited to electroporation, lipid nanoparticle-mediated delivery, polymeric nanoparticle-mediated delivery, peptide-based carrier-mediated delivery, or viral vector-mediated delivery.

In an embodiment, the engineered immune cells comprising the novel saRNA replicons described herein may be administered to a subject in need thereof for the treatment of various conditions, including cancer, inflammatory conditions, and infectious diseases.

### Genetic Engineering and Cell Modification

The saRNA constructs disclosed herein can be employed in the genetic engineering of target cells. In one embodiment, the constructs are delivered to cells ex-vivo or in-vivo to introduce genetic material, thereby enabling the cells to express one or more heterologous proteins. Methods for delivery include, but are not limited to, electroporation, lipid nanoparticle-mediated transfection, polymeric nanoparticle-mediated delivery, peptide-based carrier-mediated delivery, and viral vector-mediated delivery.

In an embodiment, the saRNA constructs described herein may be used to genetically engineer stem cells, including hematopoietic stem cells, mesenchymal stem cells, or induced pluripotent stem cells; resulting in, for example, engineered immune cells, such as T cells, natural killer (NK) cells, gamma-delta T cells, B cells, dendritic cells, or macrophages In an embodiment, these engineered stem cells may be used for regenerative medicine applications or as progenitors for the production of differentiated cells with specific therapeutic functions including for example, performing enhanced or novel functions, such as targeting specific antigens in the case of CAR-expressing cells or producing cytokines that modulate the immune response.

In an embodiment, the saRNA constructs may be co-delivered with other nucleic acids or gene-editing components, such as CRISPR-associated nucleases, to facilitate targeted modification of endogenous genes. In an embodiment, the saRNA construct may encode the gene-editing components, or these components may be delivered simultaneously using separate delivery vehicles.

In an embodiment, the saRNA constructs may be co-delivered with other nucleic acids or gene-editing components, such as CRISPR-associated nucleases, to facilitate targeted modification of endogenous genes. The saRNA construct may encode the gene-editing components, or these components may be delivered simultaneously using separate delivery vehicles.

In an embodiment, the genetic engineering methods described herein may be tailored to achieve either transient or sustained modification, depending on the specific application. In an embodiment, for transient modification, the saRNA constructs may be designed to express the desired protein for a defined period without integration into the host genome. In an embodiment, for more durable modification, the saRNA constructs may be combined with gene-editing strategies to achieve stable genomic integration or permanent alteration of endogenous genes.

In an embodiment, the genetic engineering approach involves a single round of modification using a single saRNA construct. In an embodiment, cells may undergo multiple rounds of modification using different saRNA constructs, or combinations of saRNA constructs and other genetic engineering tools, to achieve complex phenotypic changes.

### Therapeutic Applications

In an embodiment, the unique immune cells comprising the novel saRNA constructs disclosed herein are useful for therapeutic application. In an embodiment, the immune cells may be utilized to treat cancer or inflammatory conditions. As used herein the term “cancer” includes but is not limited to carcinoma, sarcoma, leukemia, lymphoma, multiple myeloma, melanoma, brain and spinal cord tumors, germ cell tumors, neuroendocrine tumors, and carcinoid tumors. As used herein, the term “inflammatory condition” includes, but is not limited to lupus, gout, psoriatic arthritis, myositis, scleroderma, rheumatoid arthritis, vasculitis, Kawasaki Disease, or eosinophilic conditions such as severe eosinophilic asthma (SEA).

In an embodiment, the immune cells comprising the novel saRNA replicons described herein may be used for the treatment of cancer. In an embodiment, the cancer may be a hematological malignancy, such as leukemia, lymphoma, or multiple myeloma, or a solid tumor, such as carcinoma, sarcoma, or melanoma. In such embodiments, targeting markers such as CD19 may be utilized as a benchmark target system to validate the platform's efficacy in CAR-expressing cells.

In an embodiment, the immune cells comprising the novel saRNA replicons described herein may be used for the treatment of infectious diseases. In an embodiment, the infectious disease may be caused by a virus, bacterium, fungus, or parasite.

In an embodiment, the immune cells comprising the novel saRNA replicons described herein may be used for the treatment of inflammatory or autoimmune diseases. In an embodiment, the inflammatory or autoimmune disease may be rheumatoid arthritis, lupus, multiple sclerosis, inflammatory bowel disease, psoriasis, or severe eosinophilic asthma (SEA) via the targeted depletion of eosinophils and delay of their regeneration using CCAR-engineered immune cells.

In an embodiment, the immune cells comprising the novel saRNA replicons described herein may be used for the treatment of genetic disorders. In an embodiment, the genetic disorder may be a monogenic disorder, such as sickle cell anemia, beta-thalassemia, or severe combined immunodeficiency.

In an embodiment, the saRNA constructs described herein encoding CCAR payloads are used for the treatment of eosinophilic diseases. The IL-5-CCAR-T cells target and eliminate IL-5Rα-expressing eosinophils and eosinophil progenitors, thereby reducing eosinophil counts in target tissues. This approach provides a durable, single-dose therapeutic option for patients with severe eosinophilic asthma, hypereosinophilic syndrome, chronic eosinophilic leukemia, eosinophilic esophagitis, chronic rhinosinusitis, and other eosinophil-driven conditions.

In an embodiment, the trivalent saRNA construct encoding IL-4-CCAR, IL-5-CCAR, and IL-4 mutein enables a multi-pronged therapeutic approach to type 2-high asthma by simultaneously: (a) depleting eosinophils via IL-5-CCAR-mediated killing; (b) targeting IL-4 receptor-expressing cells via IL-4-CCAR-mediated killing; and (c) blocking IL-4 and IL-13 signaling via secretion of IL-4 mutein. This trivalent approach addresses the multiple cytokine pathways that drive type 2 inflammation and provides more comprehensive disease control than agents targeting a single pathway.

In an embodiment, the CCAR-T cells engineered using the saRNA constructs described herein may be administered to a subject as a single infusion, providing durable control of eosinophil-driven inflammation over an extended period exceeding the typical therapeutic window of current monoclonal antibody-based treatments.

### Pharmaceutical Compositions and Methods

In an embodiment, the saRNA constructs disclosed herein may be administered in a variety of pharmaceutical compositions. In an embodiment, the saRNA is formulated with a delivery vehicle selected from the group consisting of lipid nanoparticles, polymeric nanoparticles, peptide-based carriers, viral vectors, and exosomes. In an embodiment, the delivery system may further comprise components such as an ionizable lipid, a helper lipid, cholesterol, or a PEG-lipid, which aid in targeted delivery and cellular uptake.

Moreover, the methods of use described herein include, but are not limited to, the in-vivo engineering of immune cells, methods for the treatment of cancer or inflammatory conditions, and techniques for gene editing. In one embodiment, a method for producing the saRNA construct comprises in vitro transcription, co-transcriptional capping, addition of a polyadenylation tail, purification of the saRNA, and formulation within the selected delivery vehicle.

In one embodiment of a method of treatment, the pharmaceutical composition is administered to a subject such that the saRNA construct enters target cells and directs the homogeneous expression of the therapeutic protein(s) over an extended period

### Manufacturing Methods

In an embodiment, the saRNA constructs described herein may be manufactured using in vitro transcription methods. In an embodiment, the in vitro transcription may be performed using a DNA template encoding the saRNA construct, with appropriate regulatory elements such as a T7 promoter. In an embodiment, the saRNA may be co-transcriptionally capped using cap analogs, and may be modified by incorporating modified nucleotides during transcription.

In an embodiment, the saRNA constructs described herein may be purified using methods such as lithium chloride precipitation, silica-based purification, chromatography, or filtration. In an embodiment, the purified saRNA may be formulated in an appropriate buffer for storage or delivery.

In an embodiment, the immune cells comprising the novel saRNA replicons described herein may be manufactured using ex-vivo engineering methods. In an embodiment, the cells may be isolated from a subject, engineered with the saRNA constructs using methods such as electroporation or lipid-mediated transfection, expanded in culture, and reinfused into the subject.

In an embodiment, the manufacturing process for the immune cells comprising the novel saRNA replicons described herein may include quality control steps, such as testing for cell viability, phenotype, function, and sterility.

### Delivery Systems

In an embodiment, the saRNA constructs described herein may be delivered to cells or tissues using lipid nanoparticles (LNPs). In an embodiment, the LNPs may include cationic or ionizable lipids, helper lipids, cholesterol, and PEG-lipids, formulated to enhance saRNA encapsulation and cellular uptake while minimizing toxicity.

In an embodiment, the lipid nanoparticles are formulated for systemic intravenous administration to facilitate in-vivo cellular engineering. The LNPs may be surface-modified or compositionally tuned (e.g., via specific ionizable lipid-to-helper lipid ratios) to preferentially target secondary lymphoid organs, particularly the spleen, as well as peripheral circulation. This in-vivo targeting allows the LNP-encapsulated multivalent saRNA (such as the trivalent SOLAR-SEA construct) to directly transfect circulating and splenic T-cells. By engineering T-cells directly within the patient's body, this approach circumvents the requirement for ex-vivo cell harvesting, expansion, and lymphodepleting preconditioning regimens, thereby enabling a rapid, off-the-shelf therapeutic modality for acute and chronic conditions.

In an embodiment, the saRNA constructs described herein may be delivered to cells or tissues using polymeric nanoparticles. In an embodiment, the polymeric nanoparticles may include cationic polymers, such as polyethylenimine or poly(beta-amino esters), formulated to enhance saRNA encapsulation and cellular uptake while minimizing toxicity.

In an embodiment, the saRNA constructs described herein may be delivered to cells or tissues using peptide-based carriers. In an embodiment, the peptide-based carriers may include cationic peptides, cell-penetrating peptides, or targeting peptides, formulated to enhance saRNA encapsulation and cellular uptake while minimizing toxicity.

In an embodiment, the saRNA constructs described herein may be delivered to cells or tissues using viral vectors. In an embodiment, the viral vectors may include retroviruses, lentiviruses, adenoviruses, or adeno-associated viruses, engineered to package and deliver the saRNA constructs to target cells.

### OTHER EMBODIMENTS

In an embodiment, provided herein are novel saRNA replicons comprising single-stranded RNA identified as ABLE-003 (SEQ ID NO: 1), ABLE-004 (SEQ ID NO: 2), ABLE-005 (SEQ ID NO: 3), ABLE-006 (SEQ ID NO: 4), ABLE-007 (SEQ ID NO: 5), ABLE-008 (SEQ ID NO: 6), ABLE-009 (SEQ ID NO: 7), ABLE-010 (SEQ ID NO: 8), and ABLE-011 (SEQ ID NO: 9), as well as saRNA sequences encoding multivalent therapeutic payloads (e.g., SEQ ID NO: 10, SEQ ID NO: 11, SEQ ID NO: 12), where these saRNA replicons have distinct advantages over previous RNA technologies, including enhanced protein expression, increased duration of expression, and reduced immunogenicity.

In an embodiment, the saRNA constructs described herein incorporate modified nucleotides to enhance stability and reduce immunogenicity, with modifications including, but not limited to, pseudouridine, N1-methyl-pseudouridine, 5-methylcytidine, 5-hydroxymethylcytidine, 5-methyluridine, and 2′-O-methylation.

In an embodiment, the saRNA constructs described herein comprise sequence elements derived from different alphavirus species, resulting in chimeric constructs with optimized replication and expression properties in specific cell types.

In an embodiment, immune cells comprising the novel saRNA replicons described herein exhibit decreased non-self saRNA immunogenicity, increased protein expression, and increased durability compared to immune cells comprising previous saRNA replicon technologies.

In an embodiment, the unique immune cells comprising the novel saRNA replicons described herein are engineered to attack one or more tumor-kill signals or cytokine-mediated pathways, where the cells are configured to have an armored design packed with multiple recombinant proteins capable of mounting a multi-pronged attack on a tumor cell or target inflammatory cell (e.g., eosinophils).

In an embodiment, the saRNA constructs described herein are designed to express multiple therapeutic proteins in a polycistronic manner, allowing for homogeneous expression of up to four (or more) therapeutic proteins from a single saRNA molecule, such as a trivalent IL-4 CCAR/IL-5 CCAR/IL-4 mutein payload.

In an embodiment, the dual construct system described herein, comprising a first construct with non-structural genes and a second construct with one or more genes of interest, provides advantages including larger capacity for therapeutic gene payloads and improved manufacturing characteristics.

In an embodiment, the self-amplification process of the saRNA constructs described herein results in the production of approximately 104 to 105 copies of the gene of interest from a single strand of saRNA, significantly enhancing protein expression levels compared to non-amplifying mRNA systems.

In an embodiment, the unique immune cells comprising the novel saRNA replicons described herein are useful for therapeutic applications, including the treatment of cancer and inflammatory conditions, such as severe eosinophilic asthma (SEA).

In an embodiment, the immune cells comprising the novel saRNA replicons described herein may be engineered using various delivery methods, including but not limited to electroporation, lipid nanoparticle-mediated delivery, polymeric nanoparticle-mediated delivery, peptide-based carrier-mediated delivery, or viral vector-mediated delivery.

In an embodiment, the engineered immune cells comprising the novel saRNA replicons described herein may be administered to a subject in need thereof for the treatment of various conditions, including cancer, inflammatory conditions, and infectious diseases.

In an embodiment, the saRNA construct comprises a 5′UTR that may be modified for enhanced ribosome recruitment and translation efficiency, one or more non-structural protein (NSP) genes—wherein the NSP sequences may be derived from one or more alphaviruses or other virus families—and a 3′UTR engineered for increased saRNA stability. In an embodiment, the gene of interest may encode a therapeutic protein, such as a chimeric antigen receptor (CAR), a cytokine chimeric antigen receptor (CCAR), a cytokine, an immune checkpoint inhibitor, or an enzyme. In an embodiment, the saRNA construct may further include a polyadenylation sequence and may be formulated as a polycistronic message to allow the homogeneous expression of multiple proteins.

In an embodiment, modifications may be made to the non-structural protein genes, such as specific point mutations, or substitutions derived from different viral families, to optimize performance in a range of cell types. In an embodiment, the saRNA may also incorporate a defined percentage of modified nucleotides—ranging from about 0.01% to about 25%—to reduce recognition by cellular innate sensors and enhance protein expression.

In an embodiment, embodiments may utilize an internal ribosome entry site (IRES) to decouple replicase translation from cap-dependent mechanisms, thereby permitting the use of uncapped synthetic saRNA molecules and facilitating the inclusion of additional transgenes. The constructs described herein are applicable to various pharmaceutical compositions and methods of administration, including but not limited to formulations in lipid nanoparticles and other delivery vehicles.

In one embodiment, an RNA replicon comprises a 5′ untranslated region modified to include regulatory elements that enhance ribosome binding and translation initiation without reliance on a traditional 5′ cap structure. In another embodiment, the replicon includes a polyadenylation signal and engineered UTR sequences that contribute to enhanced mRNA stability and translational efficiency.

Furthermore, embodiments include the incorporation of chemically modified nucleosides, where between 0.01% to 25% of one or more nucleosides (e.g., uracil, cytosine) are replaced with analogs such as pseudouridine, N1-methyl-pseudouridine, 5-methylcytidine, 5-hydroxymethylcytidine, or 5-methyluridine. Such modifications can reduce activation of cellular innate immune sensors and increase protein expression levels.

In some embodiments, the saRNA construct comprises non-structural protein genes derived from one or more alphaviruses, including but not limited to VEEV, CHIKV, SFV, EEEV, WEEV, RRV, SINV, MAYV, ONNV, BFV, MIDV, NDUV, GETV, AURAV, TROCV, WHAV, FMV, and HJV. The combination of sequences from different viral sources can be selected to optimize performance in specific cell types or applications.

In some embodiments, the saRNA constructs are designed to express multiple therapeutic proteins in a polycistronic manner. The polycistronic design allows for homogeneous expression of multiple therapeutic proteins within the same cell, which can enhance therapeutic efficacy compared to co-delivery of multiple separate mRNAs.

Representative experimental results indicate that the modified replicons achieve sustained protein expression for periods exceeding those observed with conventional mRNA systems, with more uniform expression profiles across target cells. These results are corroborated by in vitro transfection assays and in-vivo studies, where the replicon formulations are delivered using lipid nanoparticle systems or other advanced delivery vehicles.

Preferred embodiments may further comprise detailed experimental parameters, such as methods for in vitro transcription, co-transcriptional capping procedures, purification techniques, and cell engineering protocols. Such detailed process descriptions, along with supportive data, are provided herein to ensure a comprehensive understanding of the construction, use, and potential modifications of the saRNA system.

### Cytokine-Anchored Chimeric Antigen Receptor (CCAR) Constructs

In an embodiment, the saRNA constructs described herein are utilized to express cytokine-anchored chimeric antigen receptors (CCARs). Unlike traditional CAR designs that rely on scFv-derived extracellular binding domains, which may induce immunogenic reactions including anaphylaxis, the CCAR configuration uses a human cytokine as the extracellular target-binding domain. By utilizing an endogenous human cytokine as the targeting moiety, the CCAR constructs eliminate the risk of anti-drug antibody (ADA) formation typically generated against murine or humanized scFv sequences. This is a critical structural advantage for the treatment of chronic inflammatory conditions like severe eosinophilic asthma (SEA), where repeated administrations of the LNP-saRNA therapeutic may be required over a patient's lifetime. This design avoids the immunogenicity risks associated with xenogenetically-derived scFv domains and is also advantageous in terms of time- and resource-efficiency, as the costly large-scale screening for antibody moieties can be omitted.

In an embodiment, an IL-5-anchored CCAR comprises a chimeric receptor in which human IL-5 serves as the extracellular target-binding domain, fused to intracellular signaling domains (e.g., CD28 costimulatory domain and CD3ζ signaling domain), allowing for antigen-specific activation of T-cell killing of eosinophils in an IL-5Rα-dependent manner. The human IL-5Rα is expressed on mature eosinophils, basophils, and their progenitors, making IL-5Rα an optimal target for ablating pathogenic eosinophils across developmental stages.

In an embodiment, an IL-4-anchored CCAR comprises a chimeric receptor in which human IL-4 serves as the extracellular target-binding domain, fused to intracellular signaling domains (e.g., CD28 costimulatory domain and CD3ζ signaling domain). IL-4 receptor-expressing cells, including Raji B cells, serve as target cells for IL-4-CCAR-mediated cytotoxicity.

In an embodiment, a trivalent construct is provided comprising, on a single ABLE-003 saRNA backbone (Linear-ABLE003-TC-83-VEEV-g116u(NSP1)-E112Q(NSP2)-G31R(NSP3)): (i) a human IL-4-CCAR encoding sequence; (ii) a human IL-5-CCAR encoding sequence; and (iii) an IL-4 mutein encoding sequence, interconnected by IRES elements and linker sequences. The trivalent construct simultaneously targets the IL-5/IL-5Rα axis, the IL-4/IL-4Rα axis, and IL-13 signaling, thereby addressing multiple pathways involved in severe eosinophilic asthma and type 2-high inflammatory diseases.

In an embodiment, individual CCAR constructs are provided on the ABLE-003 backbone, including: the individual IL-4-CCAR construct (Linear-ABLE003-TC-83-VEEV-g116u(NSP1)-E112Q(NSP2)-G31R(NSP3)-human IL4-CCAR); and the individual IL-5-CCAR construct (Linear-ABLE003-TC-83-VEEV-g116u(NSP1)-E112Q(NSP2)-G31R(NSP3)-human IL5-CCAR).

In an embodiment, saRNA-encoded CCAR-T cells maintain durable killing activity at Day 10 post-engineering, whereas mRNA-encoded CCAR-T cells lose their activity. This demonstrates the significant advantage of the saRNA platform disclosed in the parent application for sustained CCAR expression and durable immune cell function when combined with the CCAR payloads newly disclosed herein.

### RNA Replicon Constructs

In an embodiment, the saRNA constructs described herein comprise a 5′ untranslated region (5′UTR), non-structural protein genes (NSP1-4), one or more genes of interest encoding therapeutic proteins, a 3′ untranslated region (3′UTR), and a poly(A) tail. In an embodiment, the 5′UTR may include elements that enhance saRNA stability and translation efficiency. In an embodiment, the non-structural protein genes may include mutations or modifications that optimize replication efficiency, reduce cytotoxicity, or enhance compatibility with the host cell machinery. In an embodiment, the 3′UTR may include elements that enhance saRNA stability or translation efficiency.

Furthermore, the therapeutic payloads described herein—including the single-gene CARs (e.g., CD19-CAR), individual CCARs (e.g., IL-4 CCAR, IL-5 CCAR), and the multivalent/trivalent payloads (e.g., IL-4 CCAR/IL-5 CCAR/IL-4 mutein)—are universally compatible with, and applicable across, all saRNA backbone architectures disclosed herein. Any of the aforementioned payloads may be operably linked to the subgenomic promoter of any of the optimized replicon backbones, including but not limited to the constructs identified as ABLE-003, ABLE-004, ABLE-005, ABLE-006, ABLE-007, ABLE-008, ABLE-009, ABLE-010, ABLE-011, and ABLE-012.

In an embodiment, the saRNA constructs described herein may include a subgenomic promoter, which drives the expression of the gene(s) of interest. In an embodiment, the subgenomic promoter may be derived from an alphavirus or may be a synthetic promoter designed to enhance expression.

In an embodiment, the saRNA constructs described herein may include internal ribosome entry sites (IRESs) or 2A peptide sequences, which allow for the expression of multiple proteins from a single RNA molecule. In an embodiment, these elements may be used to express combinations of therapeutic proteins, such as CARs, cytokines, and immune checkpoint inhibitors.

In an embodiment, the saRNA constructs described herein may include sequence elements that enhance RNA stability or translation efficiency, such as stabilizing stem-loop structures, polyadenylation signals, or sequence motifs that interact with RNA-binding proteins.

In an embodiment, the saRNA constructs described herein may include sequence elements that reduce immunogenicity, such as modifications to avoid recognition by pattern recognition receptors or to inhibit the activation of interferon-stimulated genes.

### Engineered Immune Cells

In an embodiment, the immune cells comprising the novel saRNA replicons described herein may be T cells, including CD4+ T cells, CD8+ T cells, gamma-delta T cells, or regulatory T cells. In an embodiment, the engineered T cells may express one or more therapeutic proteins, such as CARs, CCARs, T cell receptors (TCRs), cytokines, or immune checkpoint inhibitors.

In an embodiment, the immune cells comprising the novel saRNA replicons described herein may be natural killer (NK) cells. In an embodiment, the engineered NK cells may express one or more therapeutic proteins, such as CARs, CCARs, natural cytotoxicity receptors, cytokines, or immune checkpoint inhibitors.

In an embodiment, the immune cells comprising the novel saRNA replicons described herein may be B cells, dendritic cells, or macrophages. In an embodiment, these engineered cells may express one or more therapeutic proteins, such as antigens, cytokines, or co-stimulatory molecules.

In an embodiment, the immune cells comprising the novel saRNA replicons described herein may be engineered to express a combination of therapeutic proteins that enhance their anti-tumor activity. For example, in an embodiment, the cells may express a CAR targeting a tumor antigen, a cytokine that enhances immune cell function (e.g., IL-12, IL-15, IL-18), and an inhibitor of immune checkpoints (e.g., anti-PD-1, anti-CTLA-4); alternatively, for anti-inflammatory activity, the cells may express a multivalent payload such as a trivalent combination of an IL-4 CCAR, an IL-5 CCAR, and an IL-4 mutein.

In an embodiment, the immune cells comprising the novel saRNA replicons described herein may be autologous to the subject in need thereof. In an embodiment, the use of autologous cells may reduce the risk of immune rejection and enhance the persistence and efficacy of the engineered cells.

In an embodiment, the immune cells comprising the novel saRNA replicons described herein may be allogeneic to the subject in need thereof. In an embodiment, the use of allogeneic cells may provide logistical advantages, such as off-the-shelf availability and reduced manufacturing costs.

### Vector Constructs and Delivery Systems

In an embodiment, the present invention further provides a vector comprising the nucleic acid encoding the saRNA construct. In an embodiment, the vector may be a plasmid that includes a replication origin and selectable marker for propagation in host cells. Alternatively, in an embodiment, the vector may be a viral vector, such as an engineered alphavirus, wherein the RNA construct is incorporated into the viral genome. In an embodiment, such viral vectors may be rendered replication-defective or conditionally replicative to enhance safety.

In an embodiment, the vector is formulated for delivery by non-viral means. For instance, in an embodiment, the saRNA construct may be encapsulated within lipid nanoparticles or polymeric nanoparticles. In an embodiment, the vector may additionally comprise promoter sequences, enhancers, or other regulatory elements that ensure efficient transcription, replication, or expression of the therapeutic gene.

In an embodiment, the vector embodiments described herein may also be designed to permit the insertion of additional genetic elements, such as reporter genes, selectable markers, or regulatory elements that modulate expression levels. This allows for flexibility in applications ranging from gene therapy to cell engineering and vaccination.

TABLE 1
Summary of Sequences

| SEQ | Length | |
|---|---|---|
| ID NO. | (bp) | Description |
| 1 | 8,712 | ABLE-003: Linear-ABLE003-TC-83-VEEV-g116u(NSP1)- |
| | | E112Q(NSP2)-G31R(NSP3)-mCherry |
| 2 | 8,712 | ABLE-004: Linear-ABLE004-TC-83-VEEV-g116u(NSP1)-mCherry |
| 3 | 8,712 | ABLE-005: Linear-ABLE005-TC-83-VEEV-g116u(NSP1)- |
| | | E112Q(NSP2)-mCherry |
| 4 | 8,712 | ABLE-006: Linear-ABLE006-TC-83-VEEV-E112Q(NSP2)- |
| | | G31R(NSP3)-mCherry |
| 5 | 8,724 | ABLE-007: Linear-ABLE007-TC-83-VEEV-coCHIKV NSP2-mCherry |
| 6 | 8,952 | ABLE-008: Linear-ABLE008-TC-83-VEEV-5 prime and 51ntCSE- |
| | | g116u(NSP1)-E112Q(NSP2)-G31R(NSP3)-mCherry |
| 7 | 8,394 | ABLE-009: Linear-ABLE009-TC-83-VEEV-5 prime and 51ntCSE- |
| | | g116u(NSP1)-E112Q(NSP2)-G31R(NSP3)-3 prime UTR of SFV- |
| | | mCherry |
| 8 | 8,394 | ABLE-010: Linear-ABLE010-Semliki Forest Virus-WT-mCherry |
| 9 | 8,460 | ABLE-011: Linear-ABLE011-Semliki Forest Virus-with enhancer- |
| | | mCherry |
| 10 | 8,712 | ABLE-001: Linear-ABLE001-WT-VEEV-mCherry (reference) |
| 11 | 8,712 | ABLE-002: Linear-ABLE002-TC-83-VEEV-mCherry (reference) |
| 12 | 11,380 | Trivalent construct: Linear-ABLE003-TC-83-VEEV-g116u(NSP1)- |
| | | E112Q(NSP2)-G31R(NSP3)-human IL4-CCAR-human IL5-CCAR- |
| | | IL4 mutein |
| 13 | 9,191 | Individual IL-4 CCAR construct: Linear-ABLE003-TC-83-VEEV- |
| | | g116u(NSP1)-E112Q(NSP2)-G31R(NSP3)-human IL4-CCAR |
| 14 | 9,135 | Individual IL-5 CCAR construct: Linear-ABLE003-TC-83-VEEV- |
| | | g116u(NSP1)-E112Q(NSP2)-G31R(NSP3)-human IL5-CCAR |
| 15 | 1,187 | IL-4 CCAR encoding region (full-length with signaling domains) |
| 16 | 1,131 | Human IL-4 CCAR encoding sequence (from trivalent construct) |
| 17 | 1,131 | Human IL-5 CCAR encoding sequence (from trivalent construct) |
| 18 | 413 | IL-4 mutein encoding sequence |
| 19 | 45 | IRES element sequence |
| 20 | 45 | Linker sequence (GGGGSGGGGSGGGGGS) |
| 21 | 9,191 | Full IL-4 CCAR construct on ABLE-003 backbone (variant) |
| 22 | 1,190 | IL-4 CCAR encoding region (with signal peptide, variant) |
| 23 | 9,135 | Full IL-5 CCAR construct on ABLE-003 backbone (variant) |
| 24 | 1,134 | IL-5 CCAR encoding region (with signal peptide, variant) |
| 25 | 9,462 | CD19-CAR construct: Linear-ABLE003-TC-83-VEEV-g116u(NSP1)- |
| | | E112Q(NSP2)-G31R(NSP3)-human CD19-CAR |
| 26 | 1,398 | Human CD19-CAR encoding sequence |

TABLE 2
ABLE Backbone Constructs

| SEQ | | | |
|---|---|---|---|
| ID | Construct | | Reporter/ |
| NO. | Name | Key Modifications | Payload |
| 1 | ABLE-003 | g116u(NSP1), E112Q(NSP2), | mCherry |
| | | G31R(NSP3) | |
| 2 | ABLE-004 | g116u(NSP1) | mCherry |
| 3 | ABLE-005 | g116u(NSP1), E112Q(NSP2) | mCherry |
| 4 | ABLE-006 | E112Q(NSP2), G31R(NSP3) | mCherry |
| 5 | ABLE-007 | coCHIKV NSP2 chimera | mCherry |
| 6 | ABLE-008 | g116u(NSP1), E112Q(NSP2), | mCherry |
| | | G31R(NSP3), 51ntCSE | |
| 7 | ABLE-009 | g116u(NSP1), E112Q(NSP2), | mCherry |
| | | G31R(NSP3), 51ntCSE, | |
| | | SFV 3′UTR | |
| 8 | ABLE-010 | SFV-derived, wild-type | mCherry |
| 9 | ABLE-011 | Semliki Forest Virus-derived, | mCherry |
| | | with enhancer | |
| 10 | ABLE-001 | WT-VEEV | mCherry |
| | (ref.) | | |
| 11 | ABLE-002 | TC-83-VEEV | mCherry |
| | (ref.) | | |

TABLE 3
Therapeutic Payload Constructs

| SEQ | Construct/ | Length | | |
|---|---|---|---|---|
| ID NO. | Component | (bp) | Backbone | Description |
| 12 | Trivalent SEA | 11,380 | ABLE-003 | Full-length trivalent encoding IL-4 CCAR, |
| | Construct | | | IL-5 CCAR, and IL-4 mutein connected by |
| | | | | IRES elements and linker sequences |
| 13 | IL-4 CCAR on | 9,191 | ABLE-003 | Individual IL-4 CCAR on |
| | ABLE-003 | | | g116u/E112Q/G31R backbone |
| 14 | IL-5 CCAR on | 9,135 | ABLE-003 | Individual IL-5 CCAR on |
| | ABLE-003 | | | g116u/E112Q/G31R backbone |
| 16 | IL-4 CCAR | 1,131 | — | IL-4 CCAR encoding region within the |
| | encoding | | | trivalent construct |
| 17 | IL-5 CCAR | 1,131 | — | IL-5 CCAR encoding region within the |
| | encoding | | | trivalent construct |
| 18 | IL-4 mutein | 413 | — | IL-4 mutein encoding region within the |
| | encoding | | | trivalent construct |
| 19 | IRES element | 45 | — | Internal ribosome entry site connecting |
| | | | | payload elements |
| 20 | Linker | 45 | — | Flexible glycine-serine linker |
| | | | | (gggggcggtggttccggaggaggtggatctggcggaggagggtca) |
| 21 | IL-4 CCAR | 9,191 | ABLE-003 | Alternative IL-4 CCAR full construct |
| | (variant) | | | |
| 22 | IL-4 CCAR | 1,190 | — | IL-4 CCAR encoding region with signal |
| | region (variant) | | | peptide (variant) |
| 23 | IL-5 CCAR | 9,135 | ABLE-003 | Alternative IL-5 CCAR full construct |
| | (variant) | | | |
| 24 | IL-5 CCAR | 1,134 | — | IL-5 CCAR encoding region with signal |
| | region (variant) | | | peptide (variant) |
| 25 | CD19-CAR on | 9,462 | ABLE-003 | Human CD19-CAR on g116u/E112Q/G31R |
| | ABLE-003 | | | backbone |
| 26 | CD19-CAR | 1,398 | — | Human CD19-CAR encoding region |
| | encoding | | | |

TABLE 4
Nucleotide Sequence of Linker

| Position | Sequence |
|---|---|
| 1-45 | GGGGGCGGTGGTTCCGGAGGAGGTGGATCTGGCGGAG |
| | GAGGGTCA (SEQ ID NO: 20) |

TABLE 5
Trivalent Construct Organization (SEQ ID NO: 12)

| | Nucleotide Range | | Corresponding |
|---|---|---|---|
| Region | (Approximate) | Component | SEQ ID NO. |
| 5′UTR | 1-44 | VEEV 5′ untranslated | SEQ ID |
| | | region | NO: 27 |
| NSP1 | 45-1655 | Non-structural protein 1 | SEQ ID |
| | | (with g116u mutation) | NO: 28 |
| NSP2 | 1656-4052 | Non-structural protein 2 | SEQ ID |
| | | (with E112Q mutation) | NO: 29 |
| NSP3 | 4053-5498 | Non-structural protein 3 | SEQ ID |
| | | (with G31R mutation) | NO: 31 |
| NSP4 | 5499-7343 | Non-structural protein 4 | SEQ ID |
| | | | NO: 39 |
| SG | 7344-7381 | Subgenomic promoter | SEQ ID |
| Promoter | | | NO: 33 |
| IL-4 | 7382-8512 | Human IL-4 CCAR | SEQ ID |
| CCAR | | | NO: 16 |
| IRES | 8513-8557 | Internal ribosome | SEQ ID |
| | | entry site | NO: 19 |
| IL-5 | 8558-9688 | Human IL-5 CCAR | SEQ ID |
| CCAR | | | NO: 17 |
| Linker | 9689-9733 | Glycine-serine flexible | SEQ ID |
| | | linker | NO: 20 |
| IL-4 | 9734-10146 | IL-4 mutein (antagonist) | SEQ ID |
| mutein | | | NO: 18 |
| 3′UTR + | 10147-11380 | VEEV 3′ untranslated | SEQ ID |
| polyA | | region and poly(A) tail | NO: 45 |

TABLE 6
Sub-Region Sequences

| SEQ | | |
|---|---|---|
| ID | Length | |
| NO. | (bp) | Description |
| 27 | 34 | VEEV 5′UTR |
| 28 | 1,619 | VEEV NSP1 with TC-83 mutation |
| 29 | 2,382 | VEEV NSP2 wild-type |
| 30 | 1,671 | VEEV NSP3 variant |
| 31 | 1,826 | VEEV NSP3 with G31R mutation |
| 32 | 440 | Conserved VEEV sequence and 3′UTR region |
| 33 | 35 | Subgenomic (SG) promoter |
| 34 | 1,605 | NSP1 wild-type (no mutation); present in |
| | | ABLE-001, -002, -006 |
| 35 | 2,382 | NSP2 wild-type (no mutation); present in |
| | | ABLE-001, -002, -004 |
| 36 | 2,394 | Codon-optimized CHIKV-derived NSP2 chimera; |
| | | unique to ABLE-007 |
| 37 | 1,671 | NSP3 wild-type (no mutation); present in |
| | | ABLE-001, -002, -004, -005 |
| 38 | 1,671 | NSP3 from ABLE-007 (shifted 12 bp downstream |
| | | due to longer coCHIKV NSP2) |
| 39 | 1,824 | NSP4 from ABLE-007 (shifted due to |
| | | coCHIKV NSP2) |
| 40 | 35 | SG Promoter from ABLE-007 (shifted) |
| 41 | 355 | VEEV 3′UTR (standard); present in ABLE-001 |
| | | through -006 |
| 42 | 355 | VEEV 3′UTR from ABLE-007 (shifted) |
| 43 | 555 | VEEV 3′UTR (ABLE-008, extended version in |
| | | 51ntCSE construct) |
| 44 | 261 | SFV 3′UTR (from ABLE-009) |
| 45 | 1,234 | VEEV 3′UTR + polyA (trivalent construct, SEQ ID |
| | | NO: 12, nt 10147-11380) |
| 46 | 203 | 5′UTR + 51ntCSE enhancer element; present in |
| | | ABLE-008 and -009 |
| 47 | 66 | P2A self-cleaving peptide (VEEV constructs); |
| | | present in ABLE-008 and -009 |
| 48 | 85 | SFV 5′UTR; identical in ABLE-010 and -011 |
| 49 | 1,611 | SFV NSP1 |
| 50 | 2,397 | SFV NSP2 |
| 51 | 1,446 | SFV NSP3 |
| 52 | 1,845 | SFV NSP4 |
| 53 | 38 | SFV SG Promoter |
| 54 | 261 | SFV 3′UTR from ABLE-010 |
| 55 | 162 | SFV 3′UTR from ABLE-011 (shorter due to |
| | | enhancer insertion) |
| 56 | 102 | Enhancer element; unique to ABLE-011 |
| 57 | 66 | P2A self-cleaving peptide from ABLE-011 |

The following examples are given to illustrate exemplary embodiments of the present disclosure. It should be understood, however, that the present disclosure is not to be limited to the specific conditions or details described in these examples. Examples are provided below to facilitate a more complete understanding of the invention. The following examples illustrate the exemplary modes of making and practicing the invention.

### EXAMPLES

### Example 1: Design and Construction of RNA Replicons

### Protocols—Materials and Methods

The following description provides an overview of novel saRNA modifications and mutations geared towards optimizing the following attributes for the unique RNA constructs disclosed herein:
a. Increased expression compared to ABLE001 (SEQ ID NO: 10) and/or ABLE002 (SEQ ID NO: 11)
b. Increased durability of expression compared to ABLE001 (SEQ ID NO: 10) and/or ABLE002 (SEQ ID NO: 11)
c. Lower non-self RNA immunogenicity/reactogenicity against the saRNA compared to ABLE001 (SEQ ID NO: 10) and/or ABLE002 (SEQ ID NO: 11)

The designed saRNA was transcribed via in vitro transcription, transfected to cells and measured for the following—
a. Expression (% red (Fluorescent) cells OR intensity)
b. Duration of expression (defined as days until <5% of cells positive for mCherry signal)
c. Immunogenicity, as measured by interferon gamma levels (6, 24 and 48 h post transfection)

### Production of RNA Replicons Using In-Vitro Transcription—

DNA sequences encoding the 5′UTR, 3′UTR, non-structural components of alphaviral replicons, the mutations where described, and a reporter gene (e.g., mCherry) were cloned into a production plasmid between a promoter for T7 RNA polymerase and ori site. RNA was transcribed using MegaScript T7 RNA polymerase (Thermo Fisher Scientific, Waltham, MA) with co-transcriptional capping using the CleanCap trinucleotide cap 1 analog (TriLink Biotechnologies, San Diego, CA), precipitated using lithium chloride, and purified using cellulose chromatography. For sequences with nucleotide modifications, the respective residue was fully substituted with the modified nucleotide (TriLink Biotechnologies) in the reaction mixture. Resultant mRNA was analyzed by gel electrophoresis, sequenced, checked for dsRNA contaminants using a J2 dot blot, and stored frozen at −80° C. until use. The optimized T7 promoter, use of Cap 1 structure, and improved 3′UTR sequence helped with obtaining high-quality saRNA transcripts. The purification process was optimized to ensure the removal of impurities, such as template DNA and incomplete transcripts to avoid potentially impact translation efficiency or cellular responses.

Transfection of IVT RNA into Cells Using Lipoparticles

NK-92, Primary human NK-cells, BHK-21, or Primary human T-cells obtained from ATCC were grown in cell culture media as per manufacturer's recommendation and maintained in a humidified incubator at 37° C. and 5% CO2. 2 hours prior to transfection, cells were seeded at a confluency of 80% in 24-well plates. Cells were transfected with 400 ng of IVT saRNAs using commercially available liposome-based Lipofectamine™ MessengerMAX™ transfection reagent (Invitrogen, Waltham, MA, USA) at an RNA (μg): Lipofectamine™ (μL) ratio of 1:1.5. The MessengerMAX™ Reagent and the IVT RNA was diluted in two separate tubes containing 25 ul of Opti-MEM™ Medium. They were incubated for 2 minutes, and the saRNA containing mixture was added to the tube with the Lipofectamine™ to form the lipoparticle complex. After a 15 minutes incubation, the entire mixture was added to the cells.

Transfection of IVT RNA into Cells Using Electroporation

NK-92, Primary human NK-cells or BHK-21 cells, sourced from commercial entities, were grown in cell culture media as per manufacturer's recommendation and maintained in a humidified incubator at 37° C. and 5% CO2. On the day of electroporation, cells were washed with PBS, and resuspended in Buffer R to achieve a concentration of 5×10⁷ cells/ml. 1 μg of saRNA or N1-methylpseudouridine mRNA was then electroporated into 10 μl of the Buffer R resuspended cells using the Neon Electroporation system from Thermo Scientific. Electroporated cells were transferred to a 24-well plate containing pre-warmed media and maintained in cell culture incubator.

### Evaluation of Reporter Gene Expression in Transfected Cells

mCherry or GFP expression was measured using either flow cytometry or a fluorescence microscope. mCherry or GFP levels were determined starting at 6 or 24 hours post-transfection and monitored until loss of signal.

### Flow Cytometry Protocol

Cells were harvested (in duplicate samples when available), washed and stained with Live/Dead viability dye (Thermo Fisher Scientific, cat #L3244) in accordance with manufacturer's recommended procedures. Samples were then acquired on the BD FACS CANTO flow cytometer and gating analysis performed to determine the % of viable cells, the % of these viable cells that are expressing GFP and/or mCherry, and the MFI (Mean Fluorescent Intensity) for GFP and/or mCherry in positive samples.

### Microscope Analysis

Imaging of mCherry or GFP transfected cells was performed on a Invitrogen EVOS M7000 microscope. The microscope was set-up using the appropriate mCherry (RFP) or GFP filter settings, red or green channel respectively. The cell culture plates (96, 24 or 48-well) were placed under the 10× magnification lens, and images captured with optimized exposure without saturation. Un-transfected cells or WT-cells without mCherry or GFP were used as negative controls to confirm specificity.

### Cell Toxicity and Interferon Gamma Levels

6, 24 and 48 hours following saRNA transfection, media was collected and used to measure, cytotoxicity and cytokine responses. Cytotoxicity was measured using the ToxiLight™ Non-Destructive Cytotoxicity BioAssay Kit (Cat #LT07-217), Lonza, Cambridge, MA, USA. Cytokine levels were measured using the U-PLEX human Interferon Combo kit (Cat #K15094K) from Meso Scale Discovery (MSD), Rockville, MD, USA. Protocols used were as per manufacturer's recommendations.

### In-Vitro Evaluation of SOLAR Constructs

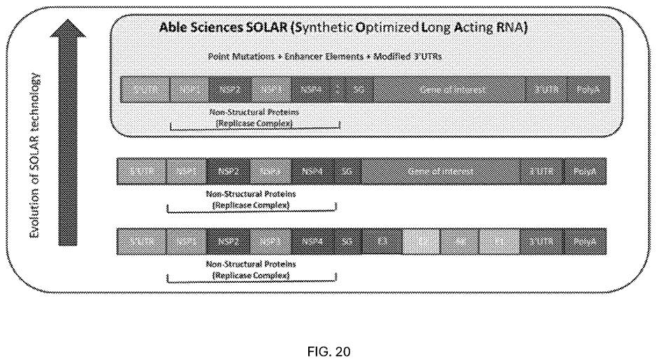

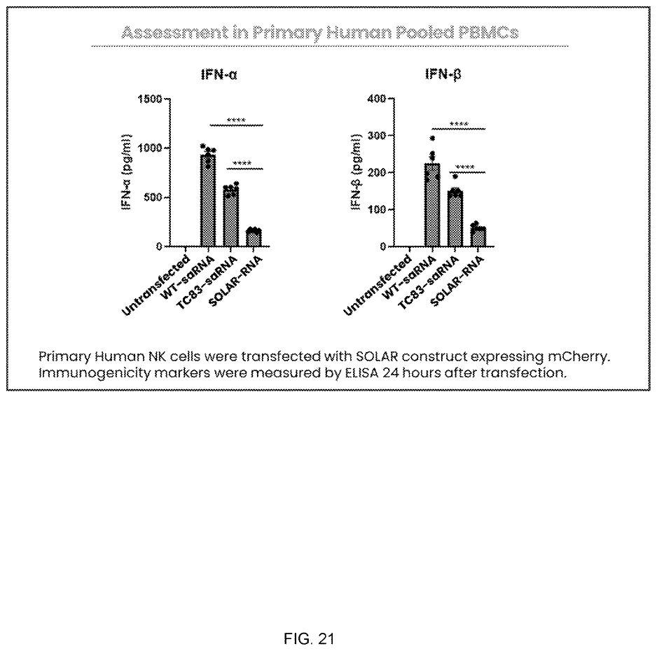

The Synthetic Optimized Long Acting RNA (SOLAR) constructs (illustrated structurally in FIG. 20) were evaluated against wild-type saRNA, TC-83 saRNA, and conventional linear mRNA to determine transcription efficiency and reactogenicity. As demonstrated in FIG. 21, transfection of primary human pooled PBMCs with the SOLAR construct elicited a statistically significant, unexpectedly lower Type-1 Interferon response (IFN-α and IFN-β) at 24 hours post-transfection compared to both WT-saRNA and TC-83-saRNA, thereby overcoming the rapid cellular shutdown typically associated with viral-derived RNA.

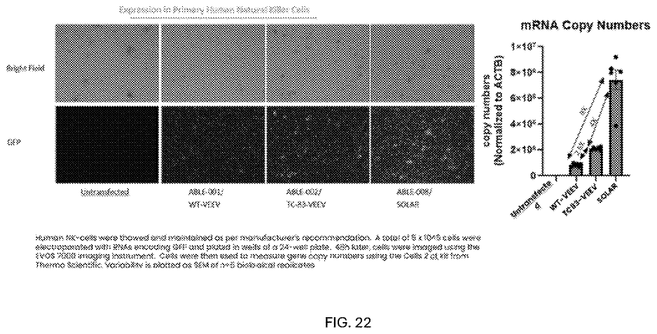

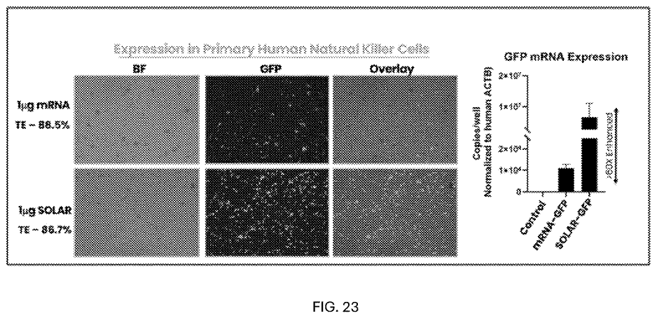

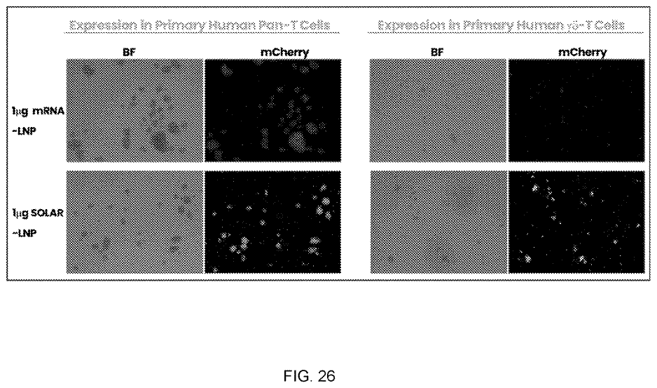

Furthermore, the SOLAR constructs demonstrated superior protein translation and RNA amplification kinetics. As shown in FIG. 22 and FIG. 23, primary human Natural Killer (NK) cells transfected with 1 μg of SOLAR-GFP exhibited a >60-fold enhancement in GFP mRNA copies per well compared to cells transfected with 1 μg of conventional mRNA, while maintaining equivalent transfection efficiencies (~86-88%). This robust expression profile is maintained across varied immune cell subtypes, including primary human Pan-T cells and primary human γδ-T cells (FIG. 26).

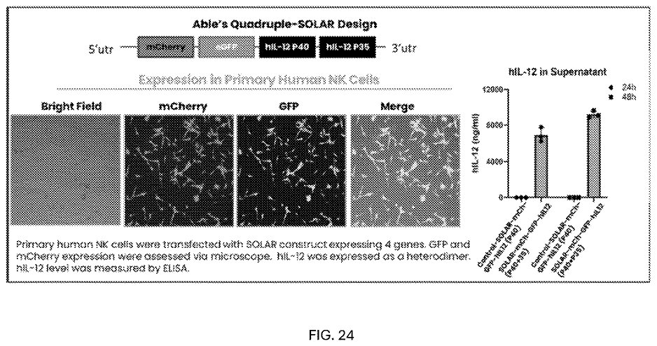

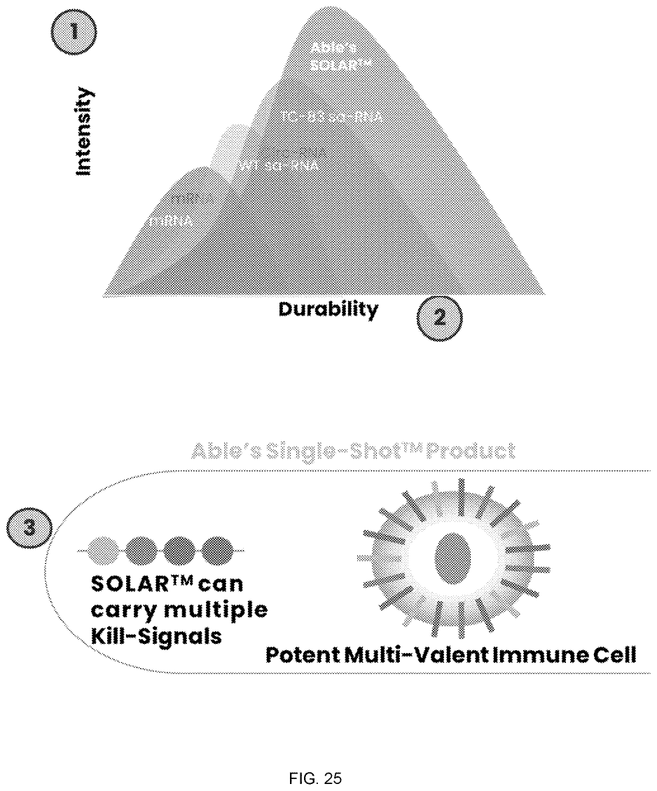

To evaluate the payload capacity of the SOLAR platform, a “Quadruple Single-Shot” multivalent construct was engineered to simultaneously co-deliver four distinct transgenes: mCherry, eGFP, and the P40 and P35 subunits of human IL-12. As demonstrated in FIG. 24, primary human NK cells successfully expressed both fluorescent reporters and secreted fully assembled, functional heterodimeric hIL-12 into the supernatant, confirming the platform's capacity to enable highly complex, multi-subunit “armored” cellular designs from a single RNA transcript.

In an embodiment, the arrangement of the distinct coding sequences within the multivalent or trivalent polycistronic constructs is entirely modular and is not limited to a fixed sequential order. For example, in an embodiment, a trivalent construct encoding an IL-4 CCAR, an IL-5 CCAR, and an IL-4 mutein may be arranged in any 5′ to 3′ configuration downstream of the subgenomic promoter. Exemplary functional arrangements include, but are not limited to: (a) 5′-[IL-4 CCAR]-[IL-5 CCAR]-[IL-4 mutein]-3′; (b) 5′-[IL-4 CCAR]-[IL-4 mutein]-[IL-5 CCAR]-3′; (c) 5′-[IL-5 CCAR]-[IL-4 CCAR]-[IL-4 mutein]-3′; and (d) 5′-[IL-4 mutein]-[IL-4 CCAR]-[IL-5 CCAR]-3′. The respective therapeutic coding regions may be separated by any suitable co-translational cleavage elements, linker sequences, or internal ribosome entry sites (IRES), such as, e.g., P2A, T2A, E2A, F2A, or encephalomyocarditis virus (EMCV) IRES—to ensure stoichiometric and homogenous translation of all components regardless of their positional order within the transcript.

### Multivalent Construct Flexibility and Alternative Configurations

The present disclosure provides multivalent polycistronic constructs with flexible component arrangements that are not limited to trivalent configurations. In an embodiment, the saRNA constructs may encode two, three, four, five, six, or more therapeutic components, with each component selected from the group consisting of chimeric antigen receptors (CARs), cytokine-anchored chimeric antigen receptors (CCARs), cytokines, cytokine muteins, immune checkpoint inhibitors, co-stimulatory molecules, and combinations thereof.

In an embodiment, a bivalent (divalent) construct is provided comprising two therapeutic components, such as: (i) an IL-5 CCAR and an IL-4 mutein; (ii) an IL-4 CCAR and an IL-5 CCAR; (iii) a CD19 CAR and IL-15; or (iv) any pairwise combination of a CCAR, a CAR, a cytokine, or a cytokine mutein. In an embodiment, the two components may be arranged in either order within the polycistronic sequence (i.e., [Component A]-[Component B] or [Component B]-[Component A]).

In an embodiment, a quadrivalent construct is provided comprising four therapeutic components. For example, a quadrivalent construct may encode: (i) an IL-4 CCAR; (ii) an IL-5 CCAR; (iii) an IL-4 mutein; and (iv) an additional therapeutic component selected from IL-15, IL-12 (as a heterodimer of P40 and P35 subunits), an additional CAR targeting a distinct antigen, or an immune checkpoint inhibitor. The four components may be arranged in any of the 24 possible permutations within the polycistronic sequence, separated by any suitable combination of 2A self-cleaving peptide sequences (e.g., P2A, T2A, E2A, F2A) or internal ribosome entry sites (IRES).

In an embodiment, a pentavalent construct is provided comprising five therapeutic components. The pentavalent construct may encode, for example: (i) an IL-4 CCAR; (ii) an IL-5 CCAR; (iii) an IL-4 mutein; (iv) IL-15 or an IL-15 superagonist; and (v) a second cytokine mutein or an immune checkpoint inhibitor. The five components may be arranged in any of the 120 possible permutations within the polycistronic sequence.

In an embodiment, a hexavalent construct is provided comprising six therapeutic components. For example, a hexavalent construct may encode: (i) an IL-4 CCAR; (ii) an IL-5 CCAR; (iii) an IL-4 mutein; (iv) IL-15; (v) a CD19 CAR or other tumor-targeting CAR; and (vi) the P40 and P35 subunits of IL-12 expressed as a heterodimer. The six components may be arranged in any of the 720 possible permutations within the polycistronic sequence. Such hexavalent constructs enable the simultaneous targeting of eosinophilic disease pathways and oncological targets within a single engineered immune cell.

In an embodiment, the therapeutic components within the multivalent constructs may be selected and combined based on the target disease or condition. In an embodiment, for eosinophilic diseases, the constructs may preferentially include IL-5 CCARs, IL-4 CCARs, and IL-4 muteins. In an embodiment, for oncological applications, the constructs may preferentially include tumor-targeting CARs (e.g., CD19, CD22, BCMA, HER2, mesothelin), cytokines that enhance anti-tumor immunity (e.g., IL-12, IL-15, IL-18), and immune checkpoint inhibitors. In an embodiment, for diseases involving both inflammatory and malignant components, hybrid constructs combining eosinophil-targeting and tumor-targeting elements may be employed.

In an embodiment, the arrangement flexibility of the multivalent constructs allows for optimization based on expression levels, protein folding, and functional activity. Without being bound by theory, the position of a therapeutic component within the polycistronic sequence may influence its translation efficiency, with components positioned closer to the 5′ end of the subgenomic transcript potentially exhibiting higher expression levels. In an embodiment, the constructs may be designed with the most critical therapeutic component positioned first in the sequence, or the arrangement may be empirically optimized for a given application.

### CAR-NK Tumor Kill Assay

In a 96-well plate, Raji-Luc2 tumor cells (target cells) were seeded at a density of 5×10³ cells/ml and allowed to attach overnight. The next day, WT-NK cells, saRNA, or mRNA-generated CAR-NK cells (effector cells), maintained for 1- or 10-days post electroporation (EP), were added to the tumor cells at various effector-to-target ratios (1:1, 3:1, 5:1, 10:1), with three replicate wells for each condition to assess dose-response. Controls included wells containing only Raji-Luc2 cells and those with only CAR-NK cells. After 24 hours of co-culture, tumor cell viability was assessed using Bright-Glo (Promega). Luminescence was measured within 10 minutes using a luminescence plate reader, and the data was normalized to the Raji-Luc2 control.

### Example 2: Additional Standard Methods

### Methods for Modified Nucleotide Incorporation

In an embodiment, modified nucleotides may be incorporated into the saRNA constructs to enhance stability and reduce immunogenicity. In an embodiment, the modified nucleotides may be incorporated during in vitro transcription by substituting the standard nucleotide with the modified variant in the reaction mixture. For example, in an embodiment, pseudouridine or N1-methyl-pseudouridine may be incorporated by replacing UTP with pseudouridine-5′-triphosphate or N1-methyl-pseudouridine-5′-triphosphate in the reaction mixture. In an embodiment, similar approaches may be used for incorporating 5-methylcytidine, 5-hydroxymethylcytidine, 5-methyluridine, and 2′-O-methylated nucleotides. In an embodiment, the incorporation efficiency and impact on RNA stability and immunogenicity may be assessed using techniques such as mass spectrometry, RNA stability assays, and cytokine release assays.

### Nanoparticle Formulation Methods

In an embodiment, for delivery of saRNA constructs to cells or tissues, lipid nanoparticle (LNP) formulations may be prepared using methods known in the art. In an embodiment, the LNP formulation comprises a cationic lipid, a helper lipid (e.g., DSPC), cholesterol, and a PEG-lipid. In an embodiment, the components may be combined in a specific molar ratio (e.g., 50:10:38.5:1.5) and mixed with saRNA in an aqueous buffer under controlled pH and temperature conditions. In an embodiment, the resulting LNPs may be characterized for size, polydispersity, encapsulation efficiency, and surface charge. In an embodiment, alternative delivery vehicles, such as polymeric nanoparticles or peptide-based carriers, may also be used following established protocols.

### Analysis of RNA Replication and Protein Expression

In an embodiment, the replication efficiency of saRNA constructs may be assessed by quantifying the copy number of RNA at various time points post-transfection. In an embodiment, total cellular RNA may be isolated using commercial kits (e.g., RNeasy Mini Kit) and subjected to quantitative RT-PCR using primers specific for the non-structural protein genes or the gene of interest. In an embodiment, the PCR data may be analyzed to determine the fold amplification of the saRNA over time.

In an embodiment, protein expression may be measured using techniques such as western blotting, ELISA, flow cytometry (for fluorescent reporters), or luciferase assays (for luciferase reporters). In an embodiment, the kinetics of protein expression, including peak expression levels and duration of expression, may be determined by sampling at various time points post-transfection. In an embodiment, for multi-cistronic constructs, the relative expression levels of different proteins may be quantified to assess the homogeneity of expression.

In an embodiment, for in-vivo studies, the saRNA constructs may be formulated in an appropriate delivery vehicle (e.g., LNPs) and administered to experimental animals via various routes, including intravenous, intramuscular, subcutaneous, or intradermal injection. In an embodiment, blood samples may be collected at defined time points to assess the pharmacokinetics of the saRNA and the expression of encoded proteins. In an embodiment, tissue samples may also be collected for analysis of biodistribution, protein expression, and histological changes. In an embodiment, for immune cell engineering applications, immune cells may be isolated from the animals post-administration to assess phenotypic and functional changes induced by the saRNA.

In an embodiment, primary immune cells may be electroporated with the saRNA in-vitro. 48 hrs post transfection, the cells are pooled and administered into animals via intravenous injection. In an embodiment, blood samples may be collected at defined time points to assess the pharmacokinetics of the saRNA and the expression of encoded proteins. In an embodiment, tissue samples may also be collected for analysis of biodistribution, protein expression, and histological changes.

### Immunological Assays

In an embodiment, the immunogenicity of saRNA constructs may be assessed using various immunological assays. In an embodiment, in vitro, the release of inflammatory cytokines (e.g., IFN-α, IFN-β, IL-6, TNF-α) from cells transfected with saRNA may be quantified using multiplex immunoassays or ELISA. In an embodiment, the activation of pattern recognition receptors (e.g., TLR3, TLR7, TLR8, RIG-I, MDA5) may be assessed using reporter cell lines or by measuring downstream signaling events. In an embodiment, in-vivo, the immunogenicity may be assessed by measuring the induction of antigen-specific T cell and antibody responses, as well as by evaluating signs of systemic inflammation.

In an embodiment, for therapeutic applications targeting tumors, the efficacy of engineered immune cells may be evaluated in tumor-bearing animal models. In an embodiment, parameters such as tumor growth, survival, tumor-infiltrating lymphocytes, and systemic immune responses may be measured to assess the therapeutic potential of the engineered cells.

### Example 3: CCAR-T Cell Functional Activity Assays (Newly Disclosed in the Present Continuation-In-Part Application)

CD19 CAR cell kill assay in Raji-Luc2 cells: CD19-CAR cytotoxic activity was evaluated using a bioluminescence-based killing assay with Raji-Luc2 target cells and effector primary human T-cells transfected with Able-saRNA-encoded CD19-CAR or mRNA-encoded CD19-CAR. Raji-Luc2 cells (expressing firefly luciferase 2) were seeded at 5,000 cells per well in a 96-well white-walled plate in complete RPMI medium. Day 1 or Day 10 post-transfected effector cells (primary T cells or engineered cell lines) expressing CD19-CAR were added at various effector-to-target (E:T) ratios ranging from 0.5:1 to 10:1, and co-cultures were incubated at 37° C., 5% CO₂ for 24 hours. Cells were lysed in 1× lysis buffer, and luciferase activity was quantified using a luminometer by adding luciferin substrate and measuring light emission (relative light units, RLU). Target cell viability was calculated relative to target-only control wells. Specific lysis was determined as follows: % cytotoxicity=[1−(RLU sample/RLU target control)]×100. Each assay condition was performed in triplicate, and results were normalized to background luminescence from assay medium alone.

IL-5 CCAR cell kill assay in primary human eosinophils: IL-5 CCAR cytotoxic activity was evaluated using a bioluminescence-based killing assay with primary human eosinophils as target cells and effector T-cells transfected with Able-saRNA-encoded IL-5 CCAR or mRNA-encoded IL-5 CCAR. Primary human eosinophils transfected with the Luc2 gene were seeded at 5,000 cells per well in a 96-well white-walled plate in complete RPMI medium. Day 1 or Day 10 post-transfected effector cells expressing IL-5 CCAR were added at various effector-to-target (E: T) ratios ranging from 0.1:1 to 5:1, and co-cultures were incubated at 37° C., 5% CO₂ for 24 hours. Luciferase activity was quantified and specific lysis was calculated as described above.

IL-5 CCAR cell kill assay in IL-5Rα-expressing A549-Luc2 cells: IL-5 CCAR cytotoxic activity was evaluated using a bioluminescence-based killing assay with IL-5Rα and Luc2 co-transfected A549 target cells and effector T-cells transfected with Able-saRNA-encoded IL-5 CCAR or mRNA-encoded IL-5 CCAR.

To further validate the receptor-specific cytotoxicity of the IL-5 CCAR constructs, EoL-1 human eosinophilic leukemia cells were utilized. EoL-1 cells endogenously express high levels of the human IL-5 receptor alpha (IL-5Rα) and serve as an established in-vitro proxy for pathogenic human eosinophils observed in severe eosinophilic asthma and chronic eosinophilic leukemia. Additionally, the use of A549 cells (a human alveolar basal epithelial cell line) engineered to transiently co-express IL-5Rα and Luc2 isolates the IL-5Rα targeting mechanism, confirming that the saRNA-encoded CCAR-T cells selectively recognize and lyse cells purely based on the presence of the targeted receptor, independent of the underlying cell type.

Target cells were seeded at 5,000 cells per well, and Day 1 post-transfected effector cells expressing IL-5 CCAR were added at various E: T ratios ranging from 0.1:1 to 5:1. Co-cultures were incubated at 37° C., 5% CO₂ for 24 hours, and target cell killing was determined by luciferase activity as described above.

IL-4 CCAR cell kill assay in Raji-Luc2 cells: IL-4 CCAR cytotoxic activity was evaluated using a bioluminescence-based killing assay with Raji-Luc2 target cells and effector primary human T-cells transfected with Able-saRNA-encoded IL-4 CCAR or mRNA-encoded IL-4 CCAR. Raji-Luc2 cells (expressing firefly luciferase 2) were seeded at 5,000 cells per well in a 96-well white-walled plate in complete RPMI medium. Day 1 post-transfected effector cells expressing IL-4 CCAR were added at various E: T ratios ranging from 0.1:1 to 5:1, and co-cultures were incubated at 37° C., 5% CO₂ for 24 hours. Luciferase activity was quantified and specific lysis was calculated as described above.

Human IL-4 mutein detection: IL-4 mutein production by cells engineered with the trivalent saRNA construct was assessed using a commercial human IL-4 ELISA kit (capture antibody-coated 96-well plate, detection antibody, streptavidin-HRP) according to the manufacturer's instructions. Recombinant IL-4 standards and samples (100 μL/well) were prepared in duplicate, added to the pre-coated plate, and incubated for 1 hour at room temperature. Samples were removed and wells were washed 3-5 times with PBS-0.05% Tween. Detection antibody (100 μL) was added, incubated for 1 hour, and wells were washed 3-5 times. Streptavidin-HRP was added and incubated 20-30 minutes. Wells were washed, and 100 μL TMB substrate was added, developed 5-20 minutes in the dark, and the reaction was stopped. Absorbance was read at 450 nm and IL-4 concentrations were determined by fitting the standard curve per kit instructions.

### Experimental Results of CCAR—T Cell Functional Activity Assays

The following experimental results demonstrate the functional activity of CAR and CCAR constructs expressed from the saRNA platform described herein.

### CD19 CAR-NK Cell Tumor Kill Assay Results

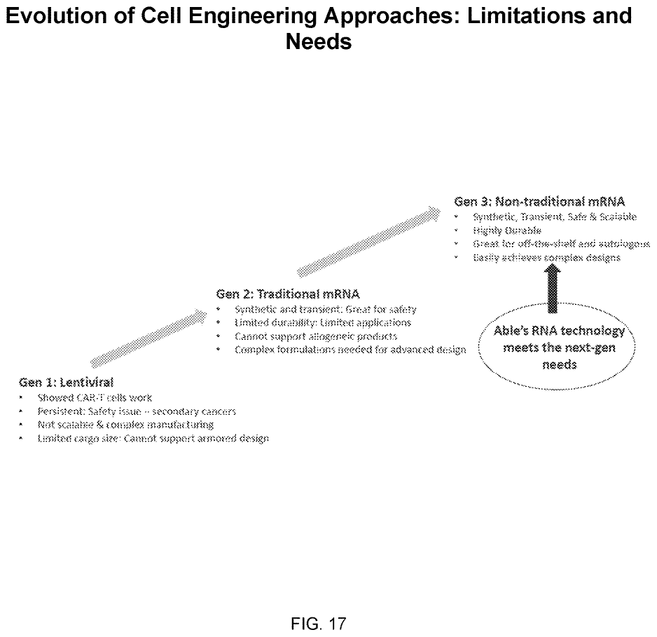

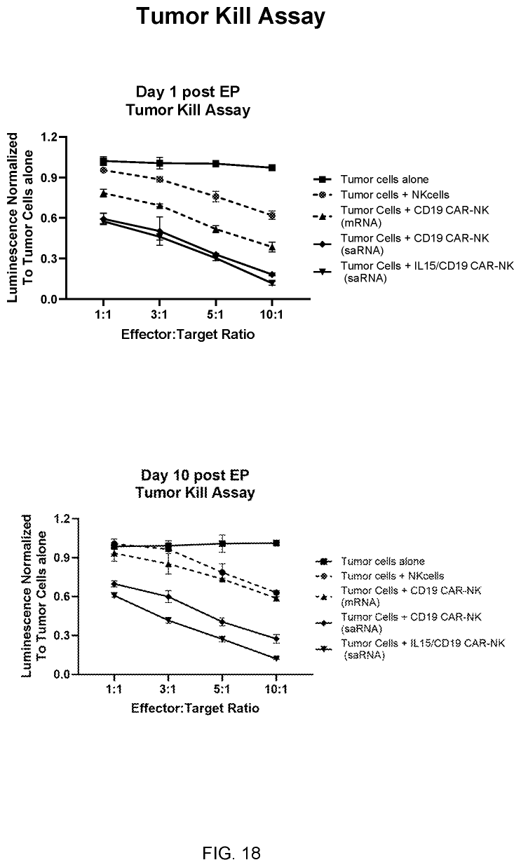

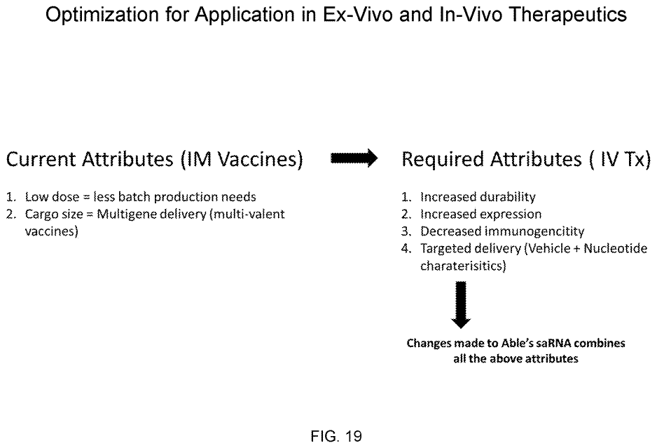

Raji-Luc2 tumor cells were co-cultured with CD-19 CAR-NK cells engineered with either mRNA-CD19 CAR or Able-saRNA CD19 CAR at effector-to-target (E:T) ratios ranging from 1:1 to 10:1. As shown in FIG. 18, at Day 1 post-engineering, both mRNA and saRNA constructs facilitated dose-dependent target lysis. However, as also demonstrated in FIG. 18, at Day 10 post-engineering, the mRNA-encoded CAR-NK cells lost substantial cytotoxic capacity, whereas the SOLAR-encoded CD19 CAR-NK cells maintained robust, durable tumor elimination. Furthermore, a dual-payload construct (SOLAR-CAR-CD19+IL15) exhibited superior specific lysis compared to the single-payload construct, validating the efficacy of the armored design SEQ ID NO: 25).

### CD19 CAR-T Cell Tumor Kill Assay Results

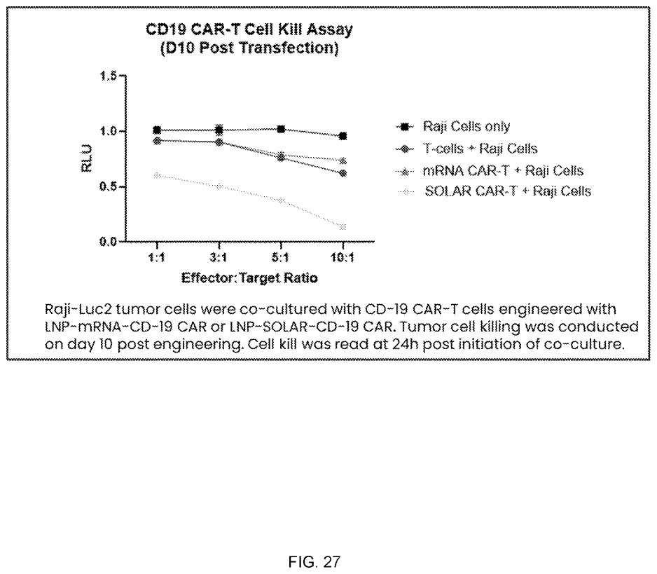

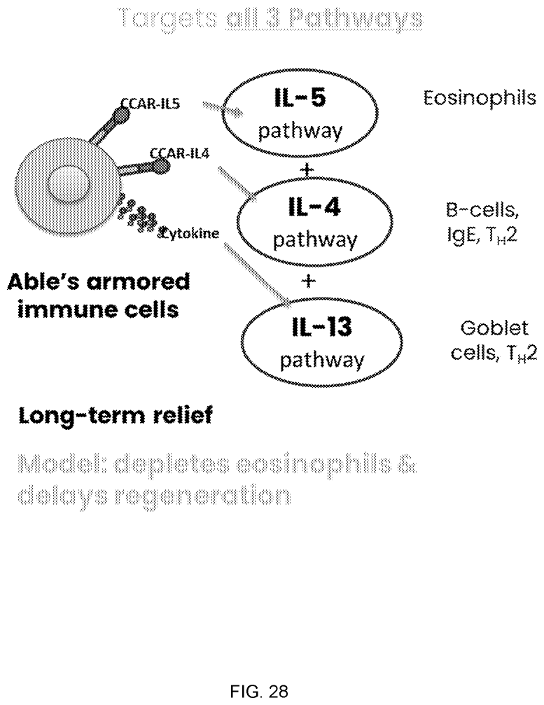

The durable cytotoxicity of the platform was similarly confirmed in primary human T-cells. As depicted in FIG. 27, SOLAR-encoded CD19 CAR-T cells maintained highly potent, dose-dependent killing of Raji-Luc2 cells at Day 10 post-engineering, outperforming the rapidly degrading mRNA-encoded equivalents.

### IL-5 CCAR-T Cell Killing of Primary Human Eosinophils

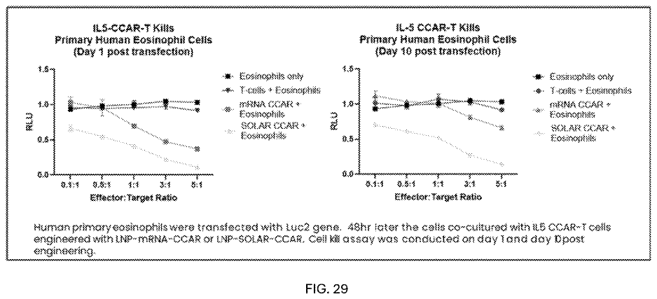

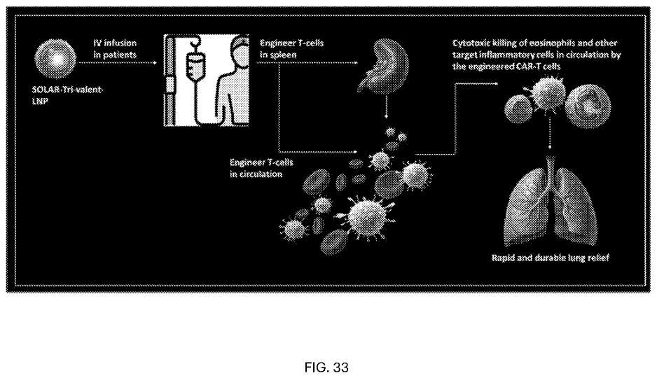

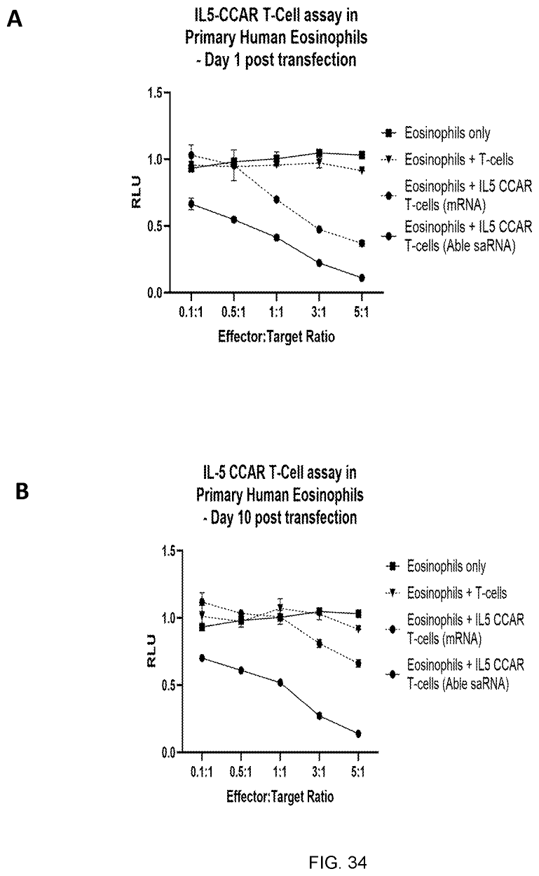

To validate the platform for severe eosinophilic asthma (SEA), primary human eosinophils (transfected with Luc2) were utilized as target cells. As shown in FIG. 29, IL-5 CCAR-T cells efficiently lysed primary eosinophils at Day 1. Crucially, at Day 10 post-engineering (FIG. 29 and FIG. 33), the SOLAR-encoded IL-5 CCAR-T cells retained substantial cytotoxic function against the primary eosinophils, while the mRNA-encoded cohort showed near-complete functional exhaustion (SEQ ID NO: 23).

### IL-5 CCAR-T Cell Killing of Cell Line Proxies (EoL-1 and A549)

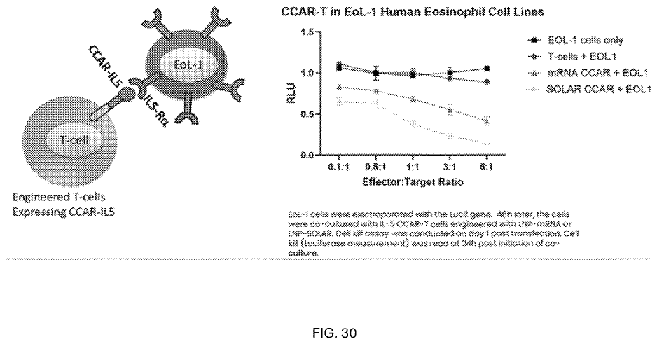

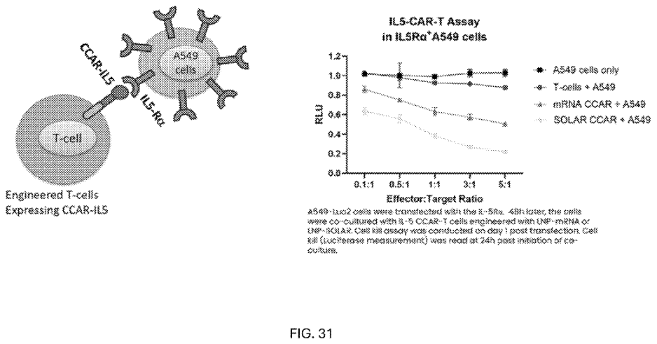

The receptor-specific mechanism of the CCAR construct was further verified using EoL-1 human eosinophilic leukemia cells (which endogenously express high levels of IL-5Rα) and A549 cells transiently co-transfected with IL-5Rα and Luc2. As shown in FIG. 30 (EoL-1 targets) and FIG. 31 (A549 targets), SOLAR-encoded IL-5 CCAR-T cells selectively recognized and lysed the target cells in a strictly IL-5Rα-dependent manner across multiple E: T ratios.

### IL-4 CCAR-T Cell Killing of Raji-Luc2 Cells

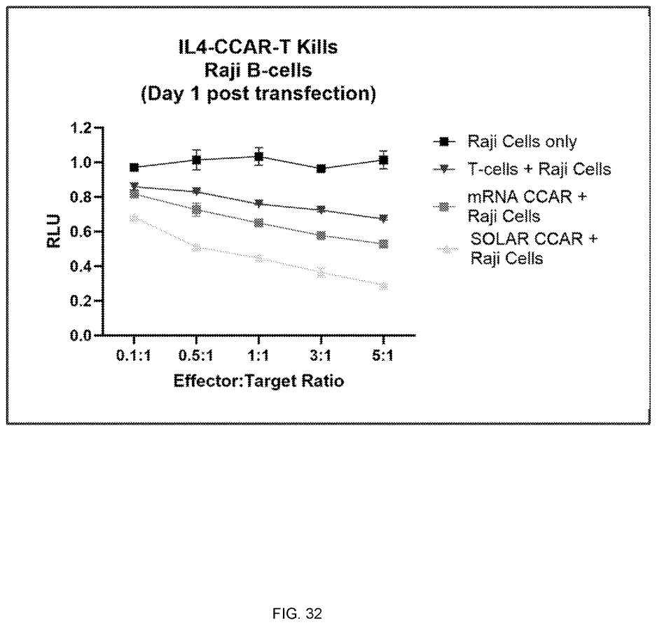

To support the multivalent targeting of secondary circulating markers in SEA, an IL-4 CCAR construct was evaluated against Raji-Luc2 cells, which natively express the IL-4 receptor. As demonstrated in FIG. 32, SOLAR-encoded IL-4 CCAR-T cells mounted a specific, dose-dependent cytotoxic response against the Raji B-cells at Day 1 post-engineering, confirming the viability of IL-4Rα as a co-target for the trivalent CCAR payload (SEQ ID NO: 21).

### Example 4: Treatment of Severe Eosinophilic Asthma Using Trivalent CCAR-T Cell Therapy

This example describes a method for treating severe eosinophilic asthma (SEA) in a human patient using engineered T cells expressing the trivalent CCAR construct disclosed herein. Patient Selection

A patient diagnosed with severe eosinophilic asthma characterized by (i) elevated blood eosinophil counts (≥300 cells/μL); (ii) inadequate disease control despite treatment with high-dose inhaled corticosteroids and long-acting β2-agonists; and (iii) history of frequent asthma exacerbations (>2 per year requiring systemic corticosteroids) is selected for treatment. The patient has previously received biologic agents targeting the IL-5/IL-5Rα pathway (e.g., mepolizumab, reslizumab, or benralizumab) or the IL-4/IL-13 pathway (e.g., dupilumab) with inadequate response or loss of efficacy.

### Cell Collection and Engineering

Peripheral blood mononuclear cells (PBMCs) are collected from the patient via leukapheresis. T cells are isolated using immunomagnetic bead selection and activated using anti-CD3/CD28 stimulation in the presence of IL-2. Activated T cells are transfected with the trivalent saRNA construct (SEQ ID NO: 12) encoding: (i) human IL-4 CCAR (SEQ ID NO: 16); (ii) human IL-5 CCAR (SEQ ID NO: 17); and (iii) IL-4 mutein (SEQ ID NO: 18), interconnected by IRES elements (SEQ ID NO: 19) and linker sequences (SEQ ID NO: 20). Transfection is performed by electroporation using optimized parameters for primary human T cells. The engineered cells are expanded in culture for 5-10 days, with CAR expression confirmed by flow cytometry using anti-IL-5 antibody staining.

### Quality Control and Release Testing

Prior to administration, the engineered trivalent CCAR-T cell product is assessed for: (i) cell viability (>70%); (ii) CAR expression (>50% of viable T cells); (iii) sterility; (iv) endotoxin levels; (v) mycoplasma testing; and (vi) in vitro potency by co-culture with IL-5Rα+ target cells. IL-4 mutein secretion is confirmed by ELISA of culture supernatants.

### Administration

In an embodiment, the trivalent CCAR-T cells are administered to the patient as a single intravenous infusion at a dose of 1×10⁶ to 1×10⁸ cells/kg body weight. Notably, unlike conventional CAR-T therapies for cancer that require lymphodepleting chemotherapy conditioning, in an embodiment, the trivalent CCAR-T cells for SEA treatment may be administered without preconditioning, as the therapeutic approach does not require complete immune reconstitution.

### Alternative In-Vivo Administration Route

In an embodiment, the requirement for ex-vivo leukapheresis and cell expansion is bypassed entirely. As illustrated in the schematic of FIG. 18, the trivalent SOLAR-SEA construct is encapsulated within a targeted lipid nanoparticle (LNP) formulated for direct systemic intravenous infusion. Upon administration, the targeted SOLAR-LNPs preferentially accumulate in secondary lymphoid organs, particularly the spleen, as well as the peripheral circulation. This facilitates the direct, in-vivo transfection and genetic engineering of circulating and splenic T-cells. The transient but highly durable nature of the SOLAR construct allows the patient's endogenous T-cells to upregulate the IL-4 CCAR, IL-5 CCAR, and IL-4 mutein payloads directly within the body, initiating rapid cytotoxic killing of circulating eosinophils and providing durable lung relief without the complex logistical burdens of traditional autologous cell manufacturing.

### Mechanism of Action

Following infusion, the trivalent CCAR-T cells are expected to exert therapeutic effects through three complementary mechanisms: (a) IL-5 CCAR-mediated killing: The IL-5 CCAR component binds to IL-5Rα expressed on mature eosinophils, basophils, and eosinophil progenitors in the blood and bone marrow, triggering T-cell-mediated cytotoxicity and eliminating the pathogenic eosinophil population. (b) IL-4 CCAR-mediated killing: The IL-4 CCAR component targets IL-4 receptor-expressing cells involved in type 2 inflammation. (c) IL-4 mutein-mediated pathway blockade: The secreted IL-4 mutein binds to IL-4Rα without inducing downstream signaling, thereby blocking both IL-4 and IL-13 signaling pathways that drive goblet cell hyperplasia, IgE production, and airway remodeling.

### Therapeutic Outcomes

Based on the saRNA platform's demonstrated durability of expression and the mechanism of the trivalent construct, the following therapeutic outcomes are anticipated: (i) rapid and sustained reduction in blood eosinophil counts to near-complete depletion; (ii) reduction in sputum eosinophils and airway inflammation; (iii) decreased serum IgE levels; (iv) decreased IL-4, IL-5, and IL-13 cytokine levels in bronchoalveolar lavage fluid; (v) improved lung function as measured by FEV1; (vi) reduced airway hyperresponsiveness; (vii) decreased frequency of asthma exacerbations; and (viii) reduced requirement for systemic corticosteroids and rescue medications.

### Duration of Effect

The durable expression characteristics of the saRNA platform, combined with the multi-pathway targeting approach of the trivalent construct, is expected to provide sustained disease control exceeding the typical therapeutic window of current monoclonal antibody-based treatments, which require repeated administration every 2-8 weeks. A single infusion of trivalent CCAR-T cells is anticipated to provide disease control for at least three months, and potentially longer, based on preclinical data demonstrating persistent protection against airway inflammation over extended periods following a single dose of IL-5-anchored CCAR-T cells.

### ALTERNATIVE EMBODIMENTS

In an embodiment, the trivalent CCAR-T cell therapy may be used for the treatment of other eosinophil-driven conditions including hypereosinophilic syndrome (HES), chronic eosinophilic leukemia (CEL), eosinophilic esophagitis, eosinophilic chronic rhinosinusitis, eosinophilic granulomatosis with polyangiitis (EGPA), atopic dermatitis, and other type 2-high inflammatory diseases. Additionally, in an embodiment, individual CCAR constructs (IL-5 CCAR alone or IL-4 CCAR alone) may be used for conditions where targeting a single pathway is sufficient.

What is claimed:

1. A self-amplifying RNA (saRNA) construct comprising a 5′ untranslated region (5′UTR), non-structural protein (NSP) genes encoding an RNA-dependent RNA polymerase complex, at least one subgenomic promoter operably linked to at least one gene of interest encoding a therapeutic protein, and a 3′ untranslated region (3′UTR), wherein the NSP genes comprise one or more modifications to enhance protein expression, increase durability, or reduce immunogenicity.

2. The saRNA construct of claim 1, wherein the construct is a polycistronic construct encoding at least two therapeutic proteins, wherein the therapeutic proteins are selected from the group consisting of a chimeric antigen receptor (CAR), a cytokine-anchored chimeric antigen receptor (CCAR), a cytokine, and a cytokine mutein.

3. The saRNA construct of claim 2, wherein the construct is a trivalent construct comprising a first CCAR, a cytokine or cytokine mutein, and a second CCAR, and wherein the arrangement of the first CCAR, the cytokine or cytokine mutein, and the second CCAR is not limited to a fixed order.

4. An engineered immune cell comprising the saRNA construct of any one of claims 1 to 3.

5. A method for treating a disease in a subject in need thereof, the method comprising administering to the subject a therapeutically effective amount of the engineered immune cells of claim 4, wherein the disease is selected from the group consisting of cancer, an eosinophilic disease, an inflammatory condition, and an autoimmune disease.
